(19) United States
(12) Patent Application Publication
(10) Pub. No.: US 2020/0078333 A1
(43) Pub. Date: Mar. 12, 2020

(54) Healthier Aging in Domesticated Animals

(71) Applicant: Richard Postrel, Miami Beach, FL (US)

(72) Inventor: Richard Postrel, Miami Beach, FL (US)

(21) Appl. No.: 16/517,556

(22) PCT Filed: Oct. 14, 2017
(86) PCT No.: PCT/US2017/056685

§ 371 Date: Jul. 20, 2019

Related U.S. Application Data
(60) Provisional application No. 62/437,739, filed on Dec. 22, 2016.
(60) Provisional application No. 62/482,196, filed on Apr. 6, 2017.

(51) Int. Cl. A61K 31/352; A61K 31/05; A61K 31/568

(52) U.S. Cl. CPC A61K 31/352; A61K 31/568; A61K 31/05

(57) ABSTRACT

This invention provides compositions, systems and methods that improve and/or optimize the health and performance of animals, especially mammals including humans, canines, equines, felines, etc. Key aspects of the principle invention and its parts include the monitoring, management and modulation of the body's natural cannabinoid systems through its native mechanisms thereby optimizing its processes by supplementation with endogenously occurring components and/or by administering synthetic compounds. The invention selects from multiple available resources to modify and/or rebalance an individual system or it may be applied across interconnected systems. The encompassing system that is intricately involved in the operations and intensities, e.g., the balancing of most other systems of our bodies, relates to cannabinoid compounds and their receptors. The cannabinoid pathways can be coordinated in rebalancing multiple and various metabolic pathways. Compositions comprising compounds that slow degradation of native endocannabinoids, that stimulate their production and/or compounds that act on cannabinoid receptors within the body are featured in systems and methods of this invention. The activated and rebalanced cannabinoid systems then become adept at modulating and the rebalancing of the numerous systems in which they participate to improve one or more systems including, but not limited to: normal and/or abnormal stresses of life, environmental influences, genomic or epigenomic irregularities and/or normal or accentuated processes of aging. Specifically targeted systems may include, but are not limited to, those involved in: cellular and/or mitochondrial metabolism, corticosteroid synthesis and effects, appetite, allergy and immunity, sleep and waking, etc. Practicing the invention selects from multiple approaches that may be used independently or in concert to compensate for a variety of concerns. Supplementing dietary intake to rebalance metabolism and/or its metabolic responses—including immunogenic or allergic responses to pathogenic and/or environmental stresses is one focus. Additional features of the invention may include: compensating for failing or decreasing androgen hormone levels as an animal continues to age, rebalancing the organism's m

### BACKGROUND

Quality of life, especially in mammals, is extended using therapies relating to aging, food allergies, pain and reduced vigor. These therapies as set forth in describing the invention rebalance molecular, cellular and subcellular biochemical requirements of mitochondria and other organelle processes

Mammals are large complex organisms made of trillions of cells. These cells comprise highly specialized differentiated cells and less differentiated cells that might act as stem cells to produce one or more highly specialized differentiated cell. The cells differentiate to maximize their abilities for assigned tasks while leaving other essential functions to other specialized cells. The cells specialize by emphasizing certain activities and eschewing others. They do this by selective induction of the proteins the individual cell makes and uses. We categorize cells, tissues, enzymes, etc. by assigning by designating them as belonging to one of more systems—i.e., groups interacting to perform related functions.

For example, the respiratory system has the task of harvesting oxygen and disposing of carbon dioxide. The respiratory system has several subsystems including the lungs and their alveoli that exchange gas molecules in air with those dissolved in blood. It also includes a part of the muscular system, the muscles that pump the air in and out of the alveoli. There is another subsystem, part of the respiratory system that provides the mucous and keeps the cell surfaces healthy and damp. The cells of the lung each have ion exchange systems that maintain cell volume and are important for muscle movement and secretions. The respiratory system is but one “system” of the complex mammalian body. Animals also need a circulatory system, a lymphatic system, a digestive system, an immune system, an integumentary system, a skeletal system, a muscular system, a nervous system, a urinary system, an endocrine system and a reproductive system. Each of these systems comprises multiple layers of subsystems. Specialized grouping of cells performing related tasks are assembled into structures called organs. Many organs and their cells can be participants in more than one system. Coordinating the various systems to act in concert for the survival of the organism is a complicated task requiring constant monitoring and rebalancing activities of the numerous subsystems and their subsystems and the individual cells that perform the systemic tasks.

Each cell itself is required to accomplish several functions. The plasma membrane separates the cell from the outside; regions within the cell, e.g., nucleus, golgi, endoplasmic reticulum, mitochondria, etc. have specialized functions that they perform. Nutrients must be internalized, while harmful or unnecessary compounds must be excluded. The right proteins must be made at the right time and delivered to the right place. Every reaction involves chemical and physical energy which must be available. The intracellular systems or activities like those of the larger organism, also involve complicated balancings and rebalancings to meet immediate needs of the cell, the organ and the animal. This system of constant rebalancings has to be almost instantaneously flexible for rapid response to the thousands of chemical reactions occurring every second. One important vector system for the control and rebalancing tasks uses several compounds, such as anandamide, 2-arachidonoylglycerol and their receptors. These were the first two compounds identified as players in our endocannabinoid system. There are several other cannabinoids now identified that bind these receptors or one of several other receptors now identified as active in the cannabinolic systems.

Mammalian cells are eukaryotic cells and therefore, like in eukaryotic organisms generally, these cells and their host organisms rely on their mitochondria to produce adenosine triphosphate (ATP). Mitochondria (and chloroplasts in plants) have their own genomes, DNA genes, that replicate independently from DNA of a cell's nucleus. But the mitochondrial genome only provides coding instruction for 1% of the proteins that are active in a mitochondrion. This is another illustration of the interdependence between the metabolic pathways in a cell. Virtually all processes of the organisms and of each of the cells of each organism require numerous overlapping pathways for continuing life and function of the organism. The overlapping pathways cannot be considered in isolation. All pathways require several inputs of energy. At a minimum, every protein involved in a pathway required energy from high energy phosphate molecules for its synthesis. The energy source most often is mitochondrially derived ATP. Enzymatic reactions are generally coupled with ATPase or GTPase activities that drive the biochemical reactions. The mitochondria themselves cannot exist in isolation. The majority of the proteins and lipids constituting the mitochondrial metabolic machinery, its transport proteins, enzymes and membranes, are provided by the healthy operation of numerous cell pathways that themselves are reliant on mitochondrial ATP production. The systems must coordinate to provide the parts to the other systems on demand. When one system malfunctions one or more back-up systems must alter their operations to meet the new needs.

Aging is a progression of serial breakdown events, generally commencing with one of the cells in a system failing to meet the needs of other cells. Each breakdown requires opportunistic remedies to address each minor failing. Cumulatively, the aging process is a gradual decline, as back-up after back-up is activated. But each back-up is less efficient than the previous with the result that performance continuously declines, the decline accelerates and vitality evaporates. When remaining back-ups are insufficient, death of cells, their tissues/organs, and eventually of the organism itself, results. Addressing the process at early stages by rebalancing nascent suboptimal systems will significantly slow or prevent the aging progression and maintain quality of life for extended periods into our prolonged years. Rebalancing the processes at earlier stages while significant back-up mechanisms remain available is more effective and by being more effective diminishes the rate that the back-up sub-systems are depleted. The present invention helps fine tune our natural systems for rebalancing and homeostatically maintaining optimal functionalities across multiple interconnected cellular, subcellular and whole organismic systems.

In each mitochondrion at the mitochondrial inner membrane, electrons from NADH and succinate are transported by the Electron Transport Chain (ETC) to oxygen, which, when it accepts the electrons, is reduced to combine with hydrogen to make water. Along the way the ETC comprises several donor and receptor enzymes in series, eventually depositing the electrons with the oxygen. Passing electrons from donor to acceptor releases energy in the form of a proton (H⁺) gradient across the mitochondrial membrane. This available ion flux has the potential to do work. This metabolic process is known as oxidative phosphorylation and results in production of adenosine triphosphate, aka, ATP. The mitochondrion is important to cell metabolism by supporting each of the cellular systems and consequently supporting survival of the organism. [Detailed descriptions of mitochondrial pathways are known or can be found in the art and need not be repeated here.]

This interdependence between the cellular systems and of the systems of cells is an illustration of the chemical and spatial communications required for each cell to maintain its life and the life of the organism. And as mentioned previously, in macroorganisms, which include, mammals, amphibians, fish, arthropods, birds, and the like, individual cells do not function independently; the skin segregates the organism from its environment; the digestive system processes fuels and eliminates food waste; the circulatory system connects far reaches of the body to control temperatures and to deliver and remove the thousands of chemicals involved in human metabolism; the endocrine and nervous systems transmit signals between distant parts of the organism; etc.

Similar interdependence exists in a cell where the plasma membrane segregates the cell from the interstitium and other cells; various organelles perform assigned functions such as DNA transcription, RNA translation, protein folding, protein degradation, energy transfer, etc. In the body and in the cell the plethora of activities cannot operate independent of one another but must be balanced to deliver needed materials to the right location at the right time to support life of each cell and of the organism. A system relying on a family of chemicals called cannabinoids and the proteins with which they interact and control plays a predominant role in this balancing.

Bodies of living organisms are in constant and continuous action responding both to external influences, e.g., temperature, sounds, light, odors, tastes, motion, society, food availability, competing organisms, commensal organisms, pathogenic organisms, etc. Animal bodies must respond appropriately to all these external influences and to externalities that the animals must internalize to provide sustenance.

As an example, cells and the host organisms require food to fuel their biologic activities. External material must therefore be internalized. This material may include alien organisms, that, like the animal and its cells, are capable of reproducing their own genomes. In response to these alien organisms attempting to use host bodies to replicate their foreign genome, large animals, require a highly coordinated and regulated immune and its associated inflammatory response to preserve good health and maintain homeostasis. However, when immune/inflammatory responses lose balance and fail to regulate themselves properly the result can be acute such as an anaphylactic shock or other allergic response or chronic disease.

Even without invasion from alien organisms, the constant and continuous stresses on living organisms require compromises in the form of opportunistic responses that optimize the cell's or more preferably the organism's modulation of, perhaps even by turning off or by restoring a slew of pathways in constant interaction to maintain the cells homeostasis at a level different from chemical and energetic equilibrium. Each system cannot be allowed to run maximally; e.g., sugar must be supplied at the proper location in amounts needed at times of need; transfer RNA (tRNA) with associated amino acids must be delivered to ribosomes to match sequences of messenger RNA (mRNA); ions must be compartmentalized and allowed to flow when needed for the numerous cell functions; etc. The thousands of biochemical reactions required every second to operate our intracellular to support its host organ's systems require signals to rapidly, almost instantaneously, switch or and off to modulate or balance the various pathways.

For example, one such system, itself with multiple pathways requiring their own controls and modulations, is the immune system, which when balanced maintains vigilance against invaders and preservers the host organism's functions. But when imbalanced by insufficient immune response may cause the organism to succumb to uncontrolled infection or when imbalanced by hyper-immune reactions can cause serious self-imposed damage to the host biomolecules, cells and possibly result in the organism's demise.

Characteristics typical of immune system imbalance may include response inadequate to kill or contain the alien substance and/or inappropriate response to non-threatening or normally benign stimuli, excitation of immune or inflammatory cells resulting in overproduction of responsive biomolecules or cells including, but not limited to: powerful oxidants (especially mitochondrial reactive oxygen species (ROS), cytokines, chemokines, eicosanoids matrix metalloproteinases, antibodies, mast cells, etc. Excessive levels of such mediators amplify allergic or inflammatory responses to degrees where they become destructive and can then be characterized as clinical symptoms.

When the internal systems become imbalanced, external influence or intervention including, for example, food components such as long chain ω-3/6 fatty acids, antioxidants, vitamins, plant flavonoids, amines, amides, sulfur compounds, iron supplement, prebiotics and probiotics are potential interventions available to us for rebalancing or modulating misdirected inflammatory or allergic situations. Macroorganisms, of course, have internal mechanisms for maintaining balance. However, these internal mechanistic paths can become overwhelmed, ineffective, misdirected, or unresponsive when external stresses are encountered. For example, viruses have adapted to recognize specific markers on cells by binding to these marker proteins as a preliminary event in the infectious process. Viral incapacitation of these proteins can elicit effects independent of the infectious process through which the viral particle co-opts the cells machinery to propagate the virus.

The 1918-19 flu pandemic (Spanish Flu) in humans resulted in over 20 million deaths, a number of deaths that exceeded those killed in the just ended World War I (or “Great War”, as it was then known). The high death rate, especially affecting healthy youths under age 26, is believed to have been caused by a cytokine storm, a hyperactive immune response.

The Spanish Flu virus, like all viral particles, self-replicates in a counterpart host. They must enter cells and commandeer the host cell's platforms to follow viral instructions for making new copies of the viral genome and its packaging. To insert its genomic material into the host cell's replication pathways, viruses require the cell to internalize the viral particle. The virus must get through the target cell's protective membrane, while leaving the cell healthy so that it can produce additional viral particles. The virus must carry a protein that will bind to and activate a protein on the target cell. The specialized binding requirements for viral entry into a cell, i.e., the necessity to bind to a specific “receptor” protein expressed on the cell membrane often limits a virus' ability to a single species or closely related species.

When cells recognize invaders, one response is suicide, a self-imposed death, often through the process called apoptosis. This may occur before viral replication thus acting to save the host organism or the virus may have ability escape recognition until it has coopted the cell to make many thousands of viral copies. These viruses have adapted so that they use later stages of the apoptosis process as an efficient means to escape the confines of the cell's plasma membrane. Disintegration of the cell membrane can effect efficient and timely release of the offspring viral particles. Healthier, younger persons were at high risk because of their robust immune systems launching a full blown immune response. The youthful healthful response to the flu infection caused release of invader fighting chemicals in such high proportions that native tissues were destroyed.

Part of the response of infected cells is to release chemicals that attract immune system cells. Reactive oxygen species (ROS) are non-specific toxic chemicals released to oxidize (chemically modify) molecules they come in contact with. Inappropriate immune response results in asthma attacks that can compromise health and is frequently fatal if the hyperactivity is not managed. Hyper-recruitment of immune cells and resulting inflammation can make breathing difficult or impossible.

Another counterproductive response often seen is “specific food allergy”, a hyperimmune response to a specific food or food biomolecule. Specific food allergy is characterized by hypersensitization to normally innocuous food proteins, lipoproteins, or other macromolecule, called the allergen. This reaction requires production of an allergen-specific IgE that binds to receptors in biomembranes of basophils and mast cells upon recurrent exposure to the same allergen. Macrophages and mast cells induce the release of inflammatory mediators, such as COX-2, iNOS, and inflammatory cytokines. Inflammatory responses have long been considered associated with the nuclear factor-kappa B (NFκB) signaling pathway.

With respect to food allergies, the gastrointestinal tract, a tract designed to encounter outside molecules and microbes, contains the largest reservoir of immune cells in the body. The function of this gastrointestinal mucosal immune system is to protect the large surface area of the gastrointestinal tract from invading pathogens and to keep the commensal microbiota compartmentalized. The mucosal immune system is divided from the gut lumen by a single layer of columnar epithelial cells, which secrete a number of biochemicals that contribute to barrier function. These include but are not limited to: mucins, antimicrobial peptides, trefoil factors, etc. The epithelial cells also transport antibodies, particularly IgA, into the intestinal lumen where these antibodies can contribute to barrier function by excluding the uptake of antigens or microbes.

Thousands of allergens have been documented for humans, including commonly, poison ivy, dust, shellfish, gluten, peanuts, etc. Common allergies in cats are against beef, seafood, and dairy products, while in dogs, chicken, beef, dairy products, eggs, wheat, and soy are common allergens.

Just with this example of the immune/allergy system of systems it is obvious that intricate balancing is required for optimal interplay between the multiple interacting and counteracting pathways to provide the weapons to dispatch invaders while sparing toxic effects on the animal's cells and systems. One fundamental balancing system that macrobiota have developed for controlling their immune responses involves the cannabinoids. Cannabinoids, chemicals that in cells are rapidly synthesized, released and degraded on demand for controlling several immunity on-off switches including one of our off-switches, corticosteroids, aka glucocorticoids.

Exogenous steroids have been used for immune suppression to counteract undesired, bothersome allergic or immune responses. In nature, low levels of corticosteroids are produced and maintained within animal cells to modulate immune system and other system responses. These steroid levels are under robust feedback control. When steroid levels are increased, endogenous synthesis virtually shuts down. Cannabinoids and cannabinoid receptors have a role in the on-off switching.

Corticosteroids are common in short term therapies used by physicians to tone down the immune system. They rapidly reduce immune and inflammatory responses for example to histamine and tumor necrosis factor. Autoimmune diseases, e.g., arthritis, are often acutely treated using hefty doses of corticosteroids with massive immune system depression as a result. These therapies however, provide strong negative feedback to the endogenous synthetic pathways. Abrupt cessations of treatment cause a dangerous shortage of steroids in circulation which can lead to severe complications, including death. While the tapering appears necessary, proper balancing with cannabinoids may facilitate the re-adaptive processes.

Steroids when used in therapy can be given by fast acting or long-lasting injection or delivered orally. But these steroids often make the recipient animal irritable so must be used with caution, especially when children may be in contact with the animal.

The two most common oral steroids are prednisone and prednisolone. Prednisone is hard for cats to metabolize and must be converted to prednisolone in the liver before it will work. Therefore, for rapid effect it is more efficient and less stressful to give prednisolone itself. In less severe and/or less acute crises, cannabinoids are viable substitutes. After the crisis is managed by prednisolone or other corticosteroid, it is advised to begin incorporating cannabinoid acting substances as either part of the weaning process or as substitute therapy.

However, in addition to this tapering issue, steroids have many dangerous side effects. Injected forms are correlated with diabetes. Steroids also damage the kidneys with extended use. The primary action of steroids is immune system suppression rapidly interrupting inflammatory reaction to the allergen. But this makes the immune suppressed recipient quite prone to infections from any advantageous pathogen gaining access to the body. Steroids also cause gastric and intestinal ulcers and prevent immunity from forming after vaccinations.

While less dramatic than anabolic steroid injection, activation of CB₁ and CB₂ results in release of epinephrine, corticosteroids, and immune calming IL-10, while decreasing pro-inflammatory IL-2. Steady supplementation with one or more synthetic or phyto-cannabinoid has effects that can be used to substitute for chronically used corticosteroid immune suppression, but will have a more natural control and fewer side effects. Cannabinoid mediated responses include a general calming of local (e.g., dermal, iliac, bowel, gastric, mucosal, etc.) pro-inflammatory mediators including, but not limited to: myeloperoxidase, CXCL8, IL-1β, TNF, etc. Cannabis has also been shown to suppress the immune system by activating myeloid-derived suppressor cells (MDSCs). MDSCs may help dampen the hyperactive immune system.

The corticosteroids are distinct from another class of steroids, the anabolic steroids. Anabolic steroids are the steroids used by athletes for growth and athletic performance. These, and in fact all anabolics (steroids or other anabolic compounds), are in a class associated with serious side effects and must be used sparingly, definitely not in the extreme doses used in the past by athletes. International sport federations ban use all anabolics at all times, during competition and even during off-season training. By contrast cannabinoids and corticosteroids (aka glucocorticoids) are classified as performance enhancing drugs and are only banned during competition. Though many anabolics are banned for use in athletic competition and only a few are approved for pharmaceutical use in many cases, use in animals may be more acceptable than use in humans.

The corticosteroids are natural hormones generated by specialized cells in the animal to tone-down immune responses and have been used clinically in animals and humans to control or even shut down immune responses. Endocannabinoids act in conjunction with the corticosteroid hormones as part of the corticosteroid pathway, but also exert their influence on several additional immuno-modulatory pathways.

In response to a viral attack on one or more cellular receptors, the immune system employs various activating and deactivating pathways for corralling and/or destroying the alien organism. Normal immune responses are balanced, i.e., the alien is dispatched by killing, walling off or removal. But more often than we would like perhaps, a hyperactive response to alien invader such as induced by the Spanish flu, or an internal (autoimmune) attack instigated by a misdirected immune system, results from significant imbalances or diversions away from healthy immunologic or other metabolic responses. Rebalancing becomes necessary for the organism's survival. An important and rapidly responsive control system used to maintain or rebalance response to external events relates to special classes of receptors on cells and organelles and delivery or the biologic signals that modulate their activities. A class of modulating biomolecules has been characterized as “endocannabinoids”. The name is derived from the receptors active in this system that also being to products from the Cannabis plant.

These compounds and the proteins responsive to them have important roles in maintaining homeostasis, especially relating to response to smells/odors, food intake, appetite, and external or internal immunologic or allergic response. The endocannabinoids were recognized as the native biomolecules that employ receptors discovered when investigating biologic responses to compounds originating in plants. Originally two cannabinoid receptors were recognized in humans/mammals because THC, a psychoactive cannabinoid substance from Cannabis was found to interact with these proteins. These were dubbed: cannabinoid receptor 1 (CB₁) and cannabinoid receptor 2 (CB₂). AEA and 2AG were recognized as predominant endocannabinoids binding these receptors. CB₁ immunoreactive neurons were found in close proximity to ileal Peyer's patches and were localized in some submucosal blood vessels. However, subsequent discoveries have revealed other endobiologic compounds also binding these receptors and the additional receptors which interact with AEA and 2AG and the additional recognized compounds with endocannabinoid activity. Homologs of human CB₁ and CB₂ receptors have been found in virtually every animal and even in plant chloroplast membranes. Most mammals tested comprise homologues for the additionally discovered cannabinoid receptors. For purposes of this discussion, the human protein designation is used.

Activation of CB₂ is generally anti-inflammatory, for example, involved in reduction of NF-κB, AP-1 and inflammatory mediators. CB₂ is primarily expressed on subsets on immune cells and several leukocyte lines of the hematopoietic subsystem (macrophages, both B and T lymphocytes), secondary lymphoid tissues such as spleen, tonsils, Peyer's patches, Lymphatic ganglia, microglia and hepatic myofibroblastic cells. CB₂ activation is a helpful pathway for turning down the immune system after an infectious disease is resolved. Two rather specific cannabinoid receptors, CB₁ and CB₂, have been identified and are targeted by numerous exogenous and endogenous cannabinoid ligands. Activation of mast cell CB₂ has direct anti-inflammatory effects, causing decreased release of pro-inflammatory mediators by these cells. Activation of CB₁ on bronchial nerve endings has bronchodilator effects acting on the airway smooth muscle with benefits for treating airway hyperreactivity and asthma. Pharmacologic interference using endocannabinoid inhibitors reduces pain and inflammation. This is mediated at least by CB₁ and CB₂. Activation of CB₁ in cerebral blood vessels has shown beneficial anti-inflammatory/anti-ischemic effects.

GPR₅₅ and CB₁ receptors modulate each other's signaling properties. GPR₅₅ forms heteromers with another 7× transmembrane spanning/GPCR which then interacts with CB₁. GPR₅₅-CB₁ heterodimer acts as a modified cannabinoid receptor that cells form to modulate activities in response to exogenous cannabinoid. This plasma membrane to response is independent of cannabinoid effects on internal organelles including, but not limited to: mitochondria, peroxisomes, endoplasmic reticulum, golgi, etc.

CB₁ and CB₂ are both expressed on Mast Cells (MC) and CB₂ is on Eosinophil (Eo) membranes, CB₁ and CB₂ have demonstrated anti-inflammatory effects on MCs. CB₁ downregulates MC degranulation, and CB₂ downregulates pro-inflammatory mediator release. Antagonizing CB₁ on the MCs stimulates degranulation and increases cell numbers without affecting MC proliferation. CB₁ activation of bronchial nerve endings has bronchodilatory effects and therefore proves to be beneficial in asthmatic response therapy. 2AG and the synthetic selective agonist JWH-133 induce Eo chemotaxis, shape change, adhesion production of reactive oxygen species and increase in CD11b expression, via CB₂ activation

Mast cells are multifunctional bone marrow-derived cells residing in mucosal and connective tissues and in the nervous system. Palmitoylethanolamide (PEA) is an endocannabinoid especially capable of downregulating MC activation and inflammation. AEA is also an effective endogenous agonist for the central cannabinoid receptor CB₁ on MCs. PEA activity may be through CB₂ and other cannabinoid receptors. PEA and AEA bind to CB₂ but AEA may be more effective when bound to CB₁. This provides evidence that PEA and/or its derivatives may be used to provide anti-inflammatory therapeutic strategies specifically targeted at MCs.

The endocannabinoid system (ECS) is an important lipid based signaling and immunomodulator system. Lipophilic compounds, those that can readily cross plasma membranes are prime activators of these endocannabinoid pathways. Research relating to medical uses of marijuana and traditional medicines has shown that at least compounds that bind CB₁ and CB₂ participate in modulating many physiological responses including, but not limited to: appetite, respiration, metabolism, inflammation, allergy, pain, neurotransmission, etc. The ECS is comprised of G-protein coupled receptors (GPCRs) including, but not limited to human: CB₁, CB₂, TRPV₁, TRPV₂, TRPV₃, TRPV₄, TRPA₁, TRPM₈, GPR₅₅, GPR₁₁₈, etc. and the animal homologues.

The native cannabinoid receptor ligands aka “endocannabinoids” are classically represented by arachidonylethanolamide (anandamide, AEA) and 2-arachidonoylglycerol (2AG). Tissue levels of endocannabinoids are maintained by the balance between biosynthesis (e.g., phospholipase D and diacylglycerol lipase-dependent and other pathways), cellular uptake and degradation by enzymes principally, but not limited to: fatty acid amide hydrolase (FAAH) and/or monoacylglycerol lipases (MAGL). Since the discovery of CB₁ and CB₂ GPCRs such as GPR₁₈, GPR₅₅, GPR₁₁₉ and the TRPs have been recognized as members of the cannabinoid family.

Exogenous AEA treatment effectively suppresses inflammatory parameters, including, but not limited to: footpad swelling, draining LN cell proliferation, histopathological cell infiltration and tissue injury. AEA decreases the induction and numbers of proinflammatory IFN-γ and IL-17 producing cells and increases IL-10 production. IL-10 acts in part through direct induction of several different microRNAs that target proinflammatory cytokines. Cannabinoid supplementation also induces populations of immunosuppressive cells including myeloid derived suppressor cells and regulatory T cells.

IL-10 was first recognized as an inhibitory factor secreted by Th2 cells that inhibited the secretion of cytokines by Th1 cells. Cannabinoids through interaction with one or more cannabinoid receptor increase expression of 1L-10. IL-10 signaling is important for inhibition of Th17 mediated inflammation.

Two notable catabolic enzymes, fatty acid amide hydrolase (FAAH) and monoglycerol lipase (MAGL), are involved in the breakdown of anandamide and 2AG, respectively. Simply put, less FAAH and MAGL means more AEA and 2AG. So inhibitors of these catabolic enzymes, for example, by nutmeg extracts, raise the levels of AEA and 2AG to generally boost cannabinoid receptor signaling. FAAH and MAGL inhibition therefore can be effective in reducing or managing pain, anxiety, hypertension and various inflammatory conditions.

The “cannabinoid” (a term indicating Cannabis-like activity) compounds have diverse effects, including most notably, some psychoactive effects became known as phytocannabinoids based on their relation to compounds found in the Cannabis genus. The endo/phyto-cannabinoids include but are not limited to: N-acylethanolami(n/d)es which include N-arachidonoylethanolamine (better known as anandamide or more simply AEA), N-palmitoylethanolamine (PEA), N-linoleoylethanolamide (LEA) and N-oleoylethanolamine (OEA). Since living organisms share many common metabolic paths and features many mammalian endocannabinoids can be found in other species, including plant species. For example, OEA and LEA are in cocoa. Black truffles when grown under certain circumstances contain high levels of AEA.

Adhesion molecules are another important target for inflammatory responses, such as asthma attack and the calming influences of cannabinoid compounds. They are an essential component for leukocyte-endothelial interactions at the inflamed site for leukocyte tethering, rolling and adhesion to the endothelium. Inactivation of adhesion molecules is associated with increased allergic/immune response. As part of the body's response to inflammatory attack, cannabinoids induce expression of adhesion molecules which have been bound, for example, ICAM-1, VCAM-1, are up-regulated on endothelial cells during ocular inflammation and CDHR3 is induced and upregulated in response to binding by RV-C. Cannabinoids have been shown to promote expression of these and other adhesion molecules in vitro.

More than 160 types of common cold viruses, aka rhinoviruses (RVs) are classified into the three species (A, B, and C) of enteroviruses within the family picornaviridae. Viruses belonging to the RV-C species were only recently differentiated in 2006. These viruses are not culturable using standard tissue-culture techniques.

Now RV-Cs have taken on highlighted clinical interest because of their involvement in severe illnesses requiring hospitalization in infants and children compared with the RV-A or RV-B. The interaction of RV-C with receptive cells is highly correlated with acute exacerbations of asthma.

Recent data reveal that the RV-C receptor is distinct from the intercellular adhesion molecule 1 (ICAM-1) that had previously been identified as the primary RV binding target and from the low-density lipoprotein receptor (LDLR) family members that bind RV-A and RV-B. Both ICAM-1 and HDCR3 act as viral receptors, but they have primary beneficial roles for the cell. Adherin proteins help organize cells into organs and tissues and control ion binding and transmembrane gradients, such as controlling the Ca⁺⁺ binding and transmembrane potential gradient.

Cadherins are a family of adhesion molecules that mediate Ca⁺⁺-dependent cell-cell adhesion in the solid tissues of multi-cellular organisms. They also modulate a variety of other processes including cell polarization and migration. Cadherin-mediated cell-cell junctions are formed by dimerization between extracellular domains of identical cadherin molecules that are located on the membranes of the adjoining cells. Stability of the adhesive junctions is enhanced by binding of the intracellular cadherin domain with the actin cytoskeleton. Several different isoforms of most cadherins exist and are distributed in a tissue-specific manner throughout most organisms. In vitro, cells containing different cadherins tend to segregate, while those that expressing same cadherin isoforms tend to aggregate together.

Cadherin-related family member 3 (CDHR3) is a human member of cadherin family of transmembrane proteins. Cadherins are cell-cell adhesion proteins requiring calcium ions (Ca⁺⁺) for adhesion. They are highly conserved in and across species. CDHR3's specific functions for the cell and organism are unknown. Cadherins in general have a significant extracellular domain that is responsible for cell-cell interactions. The extracellular portion has repeated conserved sequences for binding Ca⁺⁺, e.g., -Asp-Arg-Glu-, -Asp-Xxx-Asp- and -Asp Xxx-Asn-Asp-Ala-Pro-Xxx-Phe-. A transmembrane portion of the molecule anchors the receptor molecule to the plasma membrane. A short intracellular domain is responsible for the cadherin's intracellular signaling. The Ca⁺⁺ ions are essential for maintaining the three-dimensional structure allowing cadherins to transbind between cells and for internalizing protease recognition sites.

CDHR3 is expressed in a variety of tissues including, but not limited to: lung, liver, kidney, pancreas, thyroid, adrenal, small and large intestine, smooth and skeletal muscle, thymus, central nervous system, pancreas, marrow, etc. It is especially prominent in activity in in lung tissue bronchial epithelium, and during mucociliary differentiation in airway epithelial cells. Within a cell membrane cadherins form cis-dimers (lateral alignment). Identical cadherins on adjacent cells form trans-dimers.

Though it is not believed that the physiological reason for the CDHR3 or any other plasma membrane protein lies in its ability to bind rhinovirus (RV), this receptor is a primary recognition site for the virus to attach and gain entry into the cell. RVs are pathogens responsible for the common cold. Two specific types, RV-A and RV-C are a major agent of hospitalized infections in young children, especially those with asthma. As dog parks become more popular, the interaction and close contact between animals will likely to increase transmission of these types of viral diseases.

Cadherin receptor domains 1 and 2 may be key to virus interactions. The glycosylation, particularly at N₁₈₆, may be a contributory ligand facilitator. The conservation across species, especially including more recently diverging genuses and species strongly supports the concept that therapies will have similar effects in related classes like the mammals.

A human therapeutic antibody, Rituximab, an anti-CD20 monoclonal antibody used in humans to treat relapsed or refractory non-Hodgkin's lymphoma, has been used in dogs but Rituximab lacks sufficient affinity for action on the canine targeted B-cells. The asthmatic etiology involved with human CDCR3 includes the compound prostaglandin D2 ethanolamide, a metabolite of the endogenous cannabinoid, AEA.

Therapies involving CDHR3 can take multiple or combined paths. The virus initially decreases CDHR3 activity when it binds the receptor. This binding can lead to asthmatic symptoms, for example. However, the virus can also induce transcription and expression of the cadherin thereby making more protein available to act in its capacity to calm immune responses. Although the full pathway used by the human cadherin CDHR3 to restrain inflammatory activities is not established, the conversion of AEA to prostaglandins (PG) including, but not limited to: D2, E2, F2, G2, H2, I2, J2, etc. antagonizes the cadherin inflammation calming functions. H2 is readily converted to D2, E2, F2, I2, F1α, and thromboxanes. D2 is a major prostaglandin produced by mast cells and binds to the receptors PTGDR (DP1) and CRTH2 (DP2). This recruits Th2 cells, eosinophils, and basophils leading to an inflammatory response. D2 is a critical component in development of allergic disease responses such as asthma and therefore is of prime interest. E2/F2, I2/F1α and thromboxanes are separate production branch offshoots from H2 that can compete with D2 production.

E2 is important for labor/delivery and bone resorption. F2α is stimulated by oxytocin/pitocin and drives labor forward. Both E2 and D2 can compete for the predominant F2 receptor. I2 (aka prostacyclin) is an intermediate in F1α synthesis and is an anti-dotting agent and vasodilator in its own right. H2 is mostly known for its major intermediate status as a source for the other PGs and thromboxanes. Oxytocin also has an effect to increase AEA synthesis thereby helping to maintain balance throughout multiple systems within an animal's body.

The cadherin anti-inflammatory activity can also be maintained by using means to decrease D2 ethanolamide production. Several paths are available here. 1) the COX enzymes responsible for creation of D2 can be inhibited with NSAIDS and other COX inhibitors. 2) Breakdown of AEA can be controlled by inhibiting FAAH. 3) An engineered virus or a viral capsule without its genome can bind cadherins to induce expression. And 4) an anti-cadherin antibody, antibody fragment, or other binding agents can be used as an inductive agent. When using an inductive agent, to avoid an initial drop in cadherin modulation of the immune response, one of the other strategies may be advantageous. These strategies may be repeated and/or cycled as appropriate, for example, the viral particle may be intranasally administered one or more times, e,g., weekly, monthly, quarterly, biannually, annually, etc., with continuous inhibitor administration by oral means. The oral dose may be increased around the time of the inducement.

AEA metabolizes to Prostaglandin D2 ethanolamide through lipoxygenase and cyclooxygenase catalyzed reaction. Maintaining AEA and like compounds at effective levels and preventing metabolism to harmful products is one means for facilitating cannabinoids' balancing functions.

URB597 inhibits FAAH, the principle enzyme involved in degrading the lipid molecule AEA into its arachidonoyl and ethanolamide components. FAAH is a significant step in the pathway for creating prostaglandin ethanolamide compounds including D2 ethanolamide. Inhibiting FAAH raises natural AEA levels and leads to long-term cannabinoid receptor activation and pain relief. URB937, is another p-hydroxyphenyl-O-arylcarbamate that targets FAAH. FAAH is also responsible for the metabolism of other fatty acid amides e.g., N-oleoylethanolamine (OEA) and N-palmitoylethanolamine (PEA). FAAH inhibition maintains or increases tissue levels of anandamide in vivo. FAAH inhibitors decrease pain in rodent models of OA

COX-2 is involved in the D2 ethanolamide synthetic pathway following FAAH activity to provide both ethanolamide and substrate for D2 synthesis. There is also evidence that COX 2 may act directly on AEA in the absence of FAAH catabolism. COX-2 inhibition can serve as an alternative to FAAH inhibition to preserve the anti-allergic effects downstream of CDHR3 and may also act in conjunction in a parallel or serial treatment.

FAAH inhibitors are known in the art. Examples include but are not limited to: AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A, KML29, MAFP, MJN110, ML30, N-arachidonoyi maleimide, OL-135, OL92, PF-04457845, SA-57, ST4070, URB880, URB937, etc.

COX inhibitors are numerous and known in the art. Several are recognized as efficient inhibitors of COX-1, others are more efficient inhibiting COX-2. Many inhibit both COX-1 and COX-2 or their selectivities are riot well-established. COX-1 inhibitors include but are not limited to: indomethacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen (favors COX-1), TFAP, etc. While COX-2 inhibitors include but are not limited to: 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)indomethacin amide, indomethacin heptyl ester, SC236, etc. And non-specific or those without clear specificity include but are not limited to: COX sulinac, sulindac sulfide, pravadoline, naproxen, naproxen sodium salt, meclofenamate sodium, ibuprofen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZLI-6, isoicam, dipyrone, YS121, MEG (mercaptoethylguanidine), etc.

Managing allergic reactions in mammals is a difficult endeavor since the pool of possible allergens is immense and testing using skin patch or other common tests protocols requires considerable patient participation and can be difficult to manage in children especially in many pets. Managing by toning down or preventing continued allergic or inflammatory responses at an early common stage in the pathway before significant symptoms accrue is desired. Preventing inactivation or removal of proteins that statically suppress immune reactions can be an important aspect of this management.

The CDHR3 receptor is highly conserved throughout the animal kingdom. HDCR3 when present operates to tone-down immune responses. But, when inactivated massive allergic and/or general immense responses can result. The cadherin proteins with their common viral receptor pathways are only one example of several important proteins in pathways for managing allergic and immune responses. When control of any one or more of these is compromised thereby removing normal constraints, allergy or perhaps an asthmatic attack results.

Maintaining AEA activity and preventing its metabolism to damaging inflammatory substances is a powerful tool. COX inhibition can limit prostaglandin D2 ethanolamide formation. A selective COX inhibitor is one approach when constraints are preferred or when other COX-1 or COX-2 pathways are favorably spared. The CDHR3 downstream effects can also be maintained with FAAH inhibition. In many examples both COX inhibition and FAAH inhibition are used.

Conceptually the FAAH and COX inhibitors may be used interchangeably, with one substituted for the other or they may be used together for additive or synergistic effect. Actual selection is based on any desired factor, including, but not limited to: taste tolerance, cost, solubility, bioavailability, regulatory burden, color, consumer acceptance, etc.

### EXAMPLE 1

An animal, e.g., canine, feline or equine, receives supplements that maintain cadherin downstream activity. These supplements may be in small doses in frequent treats or on a timed schedule. The supplements inhibit downstream cadherin activity breakdown and thus maintain the strength of cadherin immune and inflammation management. COX, FAAH and/or MAGL inhibitors may be used in conjunction with or as alternatives to administering one or more cannabinoid compounds to the recipient animal.

Cadherins

Cadherins like the human CDHR3 are a family of adhesion molecules that mediate Ca⁺⁺-dependent cell-cell adhesion in all solid tissues of the animal and which modulate a wide variety of processes including cell polarization and migration. Cadherin-mediated cell-cell junctions are formed by interaction between extracellular domains of identical cadherins, which are located on the membranes of the neighboring cells. The stability of these adhesive junctions is ensured by binding of the intracellular cadherin domain with the actin cytoskeleton. There are a number of different isoforms distributed in a tissue-specific manner in a wide variety of organisms. Cells containing different cadherins tend to segregate in vitro, while those that contain the same cadherins tend to preferentially aggregate together. This observation is linked to the finding that cadherin expression causes morphological changes involving the positional segregation of cells into layers, suggesting they may play an important role in the sorting of different cell types during morphogenesis, histogenesis and regeneration. They may also be involved in the regulation of tight and gap junctions, and in the control of intercellular spacing. Cadherins are evolutionary related to the desmogleins which are component of intercellular desmosome junctions involved in the interaction of plaque proteins.

Structurally, cadherins comprise a number of domains: classically, these include a signal sequence; a propeptide of around 130 residues; a single transmembrane domain and five tandemly repeated extracellular cadherin domains, 4 of which are cadherin repeats, and the fifth contains 4 conserved cysteines and a N-terminal cytoplasmic domain. Proteins are designated as members of the broadly defined cadherin family if they have one or more cadherin repeats. A cadherin repeat is an independently folding sequence of approximately 110 amino acids that contains motifs with the conserved sequences DRE, DXNDNAPXF, and DXD. Crystal structures have revealed that multiple cadherin domains form Ca⁺⁺-dependent rod-like structures with a conserved Ca⁺⁺-binding pocket at the domain-domain interface. Cadherins depend on calcium for their function: calcium ions bind to specific residues in each cadherin repeat to ensure its proper folding, to confer rigidity upon the extracellular domain and is essential for cadherin adhesive function and for protection against protease digestion.

Prostaglandin D2 ethanolamide (PGD2-EA) is a bioactive lipid produced by the sequential metabolism of anandamide (arachidonoyi ethanolamide) by cyclooxygenase (COX) enzymes, in particular by COX-2, and PGD synthase. The biosynthesis of PGD2-EA from AEA is also increased when AEA metabolism is diminished by deletion of fatty acid amide hydrolase in experimental animals. PGD2-EA is inactive against recombinant prostanoid receptors, including the D prostanoid receptor. It increases the frequency of miniature inhibitory postsynaptic currents in primary cultured hippocampal neurons, an effect which is the opposite of that induced by AEA. PGD2-EA also induces apoptosis in colorectal carcinoma cell lines. Loss of the human cadherin CDHR3 correlates with increased inflammatory PGD2 activity. Preventing or minimizing the anti-inflammatory deficit caused by cadherin loss can restore the systems to the pre-infectious attack status.

The human CDHR3 is a member of a cadherin family of transmembrane proteins that is highly expressed in human lung tissue, bronchial epithelium, and during mucociliary differentiation in human airway epithelial cells in vitro. Members of this family interact with like cells through calcium-dependent interactions. Cadherins on the same cell surface self-associate into cis-dimers (sideways or lateral dimers). Cell adhesion holding cells together uses interactions between identical cadherins on neighboring cell surfaces to form transdimers. Modeling of the CDHR3-virus complex shows a probable binding site for monomers and dimers in a region of the RV-C15 capsid, a site that is highly conserved among different RV-C types. The docking exercise further suggests that receptor domains 1 and 2 may be key to virus interactions, and that glycosylation, particularly at Asn₁₈₆, may have some involvement. Such models of proteins and their interactions will help guide studies using mutational analysis to accurately map virus binding domains in CDHR3. Expression of CDHR3 increases RV-C binding and enables replication in host cells. Human CDHR3 thus is a functional receptor for RV-C.

A large portion of the response to RV-C attack relate to down-regulation of the HDCR3 viral receptor. Presumably, the viral receptor has another purpose and in fact this plasma membrane protein and other cadherins and other adhesive proteins work through endocannabinoids to among other functions balance allergic and immune responses. HDCR3 is not alone in these responses. The entire cadherin family likely shares similar regulatory paths in cells. The endocannabinoids therefore would be expected to have major IC involvement in a wide variety of cell interactions especially with respect to allergic and other immune system reactions.

Phytochemicals (substances found in plants or derivatives of the plant chemicals) or the plants themselves, have been recognized to possess biological activities in traditional medical practices. Several classes of compounds with similarities in structure and/or activities to the THC purported active ingredient of the marijuana source plant have been identified. These are available in several plants outside the Cannabis genus and can be, cultured (e.g., through selective breeding or genetic engineering), extracted, purified or synthesized chemically de nova or from derivatives. Such compounds including, but not limited to:
Cannabigerol class: cannabigerolic acid (CBGA) (antibiotic); cannabigerolic acid monomethylether (CBGAM); cannabigerol (CBG) (antibiotic, antifungal, anti-inflammatory, analgesic); Cannabigerol monomethylether (CBGM); cannabigerovarinic acid (CBGVA); Cannabigerovarin (CBGV).
Cannabichromene class: Cannabichromenic acid (CBCA); Cannabichromene (CBC) (antibiotic, antifungal, anti-inflammatory, analgesic); Cannabichromevarinic acid (CBCVA); Cannabichromevarin (CBCV); Cannabidiolic acid (CBDA) (antibiotic); Cannabidiol (CBD) ((antioxidant, anxiolytic, antispasmodic, anti-inflammatory, analgesic); cannabidiol monomethylether (CBDM); cannabidiol C₄ (CBD-C4); cannabidivarinic acid (CBCVA); cannabidivarin (CBDV); cannabidiorcol (CBD-C1); Δ⁹-tetrahydrocannabinolic acid A (THCA-A); Δ⁹-tetrahydrocannabinolic acid B (THCA-B); 6a,10a-trans-6a,7,8,10a-tetrahydro-6,6,9-trimethyl-3-pentyl-6H-dibenzo[b,d]pyran-1-ol, (Δ⁹ tetrahydrocannabino-, THC) (analgesic, antioxidant, antiemetic, anti-inflammation); Δ⁹-tetrahydrocannabinolic acid-C4 (THCA-C4); Δ⁹-tetrahydrocannabinol-C4 (THC-C4); Δ⁹-tetrahydrocannabivarinic acid (THCVA); Δ⁹-tetrahydrocannabivarinic (THCV); Δ⁷-cis-isotetrahydrocannabivarin; Δ⁹-tetrahydrocannabiorcolic acid (THCA-C1); tetrahydrocannabiorcol (THC-C1).
Δ⁸-tetrahydrocannabinol class: Δ⁸-tetrahydrocannabinolic acid (Δ⁸-TCA); Δ⁸-tetrahydrocannabinol Δ⁸-THC).
Cannabicyclol class: cannabicyclol (CBL); cannabicyclolicacid (CBLA); cannabicyclovarin (CBLV).
Cannabieson class: cannabiesoic acid A (CBEA-A); cannabiesoic acid B (CBEA-B); cannabieson (CBE).
Cannabinol and cannabinodiol class: cannabinolic acid (CBNA); cannabinol (CBN); cannabinol methylether (CBNM); cannabinol-C4 (CBN-C4); cannabivarin (CBV); cannabinol-C2 (CBN-C2); cannabiorcol (CBN-C1); cannabinodiol (CBND); cannabinidivarin (CBDV).
Cannabitriol class: cannabitriol (CBT); 10-Ethoxy-9-hydroxy-Δ-6a-tetrahydrocannabinol (10-EHDT); 8,9-dihydroxy-delta-6a-tetrahydrocannabinol (8,9-DHDT); cannabitriolvarin (CBTV); ethoxy-cannabitriolvarin (CBTVE).
Miscellaneous class: dehydrocannabifuran (DCBF); cannabifuran (CBF); cannabichromanon (CBCN); cannabicitran (CBT); 10-oxo-Δ-6a-tetrahydrocannabinol (OTHC); Δ⁹-cis-tetrahydrocannabinol (cis-THC); 3,4,5,6-tetrahydro-7-hydroxy-α-α-2-trimethyl-9-n-propyl-2,6-methano-2H-1-benzoxocin-5-methanol (2H-iso-HHCV); cannabiripsol (CBR); Trihydroxy-Δ⁹-tetrahydrocannabinol (triOH-THC).

LEA, PEA and OEA will bind to one or more of the endogenous cannabinoid receptors, but they are also important because they maintain AEA activity through their inhibition of the FAAH enzyme that is responsible for degrading AEA. N-alkylamides exert selective effects on the CB₂, and have been shown to exert anti-inflammatory effects similar to AEA. Echinacea contains multiple N-alkylamides that have mimetic effects.

Phytoalkanes, another class of chemical compounds found in various plants, also have demonstrated cannabinolic modulation traits, e.g., n-alkanes ranging from C₉ to C₃₉, 2-methyl-, 3-methyl-, and some dimethyl alkanes are common in spices such as curcumin. The major alkane present in an essential oil obtained by extraction and steam distillation was the n-C₂₉ alkane nonacosane (55.8 and 10.7%, respectively). Other abundant alkanes were heptacosane, 2,6-dimethyltetradecane, pentacosane, hexacosane, and hentriacontane. Curcumin reduces liver fibrosis by modulating cannabinoid receptor transmission.

In general, many plant species, especially those used for spices have anti-allergy/anti-inflammatory activities. E.g., nutmeg interacts with the endocannabinoid system by inhibiting certain key enzymes that catabolize (break down) the two main endocannabinoids, anandamide and 2AG.

β-caryophyllene, a phytocannabinoid, and/or its oxides act as full agonists of the CB₂-receptor where they exert anti-inflammatory and analgesic effects that are mediated through CB₂, but not CB₁. Another phytocannabinoid, salvinorin A, from the plant species Salvia divinorum extract is a terpenoid that interacts with a cannabinoid receptor, not yet characterized that apparently forms only in inflammatory conditions. This uncharacterized receptor also acts as a κ-opioid receptor. Many sages produce similar compounds with some activity, but whose activities have not been followed in detail to identify receptor interactions. Myrcene is a major constituent of the essential oil of hops and appears to be related to opioid “high” possibly by agonizing opioid receptors or possibly by antagonizing opioid degradation. Plant sources are hops, verbana and Cannabis. Myrcene is also found in lemongrass, thyme and mango. Echinacea contains multiple N-alkylamides that have cannabinoid mimetic effects.

The Helichrysum umbraculigerum, aka woolly umbrella Helichrysum or kerriekruie in Afrikaans, is a fast growing perennial herb with a strong mood-stabilizing and anti-depressant effect due to high concentrations of cannabigerol (CBG). Liverwort contains large amounts of perrottetinenic acid, a THC, mimetic that binds CB₁. The cacao plant has endocannabinoid activity by deactivating the FAAH enzyme thereby maintaining AEA levels and levels of similarly active fatty acid derived molecules. FAAH inhibition combines anti-inflammatory effects of several N-acylethanolamines while it targets additional receptors such as TRPV1 and peroxisome proliferator activated receptors.

URB597 is a potent and selective FAAH inhibitor. Inhibiting the FAAH enzyme, a principle degradative enzyme and one involved in synthetic pathways for inflammatory prostaglandins, maintains beneficial cannabinoid levels while reducing adverse effects from breakdown products.

Flavonoids are one of the largest nutrient families known and include over 6,000 already-identified family members. Many flavonoid classes comprise chemical members that have been tested and found to work with the cannabinoid anti-immune and ant-allergic systems. At least 20 of these flavonoid compounds, including, but not limited to: apigenin, quercetin, cannflavin A and cannflavin B, β-sitosterol, vitexin, isovitexin, kaempferol, luteolin and orientin have been identified in the Cannabis plant. Most if not all of these flavonoids are not exclusive to Cannabis also produced and harvestable/extractable from other plants. Flavonoids are known for their antioxidant and anti-inflammatory health benefits, as well as their contribution of vibrant color to the many of the foods we eat (the blue in blueberries or the red in raspberries).

Perrottetinenic acid, a phyto- or synthesized compound similar to THC appears to act through CB₁ with effects similar to AEA.

Ginger has anti-inflammatory properties without many of the side effects of synthetic pharmaceuticals. Ginger comprises several powerful anti-oxidants and at least one phyto-cannabinoid that reacts with same receptors as capsaicin (pepper), gingerol. Ginger comprises potent compounds that are efficacious agonists of the VR1 receptor. The activity of specific gingerols depends on the length of the carbon chain, which also determines hydrophobicity (partitioning between the lipid membrane and internal or external aqueous environments). Increasing the number of carbons from 6 to 8 and the hydrophobicity index from 1.90 to 2.88 in the transition from [6]-gingerol to [8]-gingerol increases by 10-fold the potency for inducing trans-plasma membrane currents (ion fluxes).

Turmeric contains the four major curcuminoids: curcumin, demethoxycurcumin, bisdemethoxycurcumin, and cyclocurcumin. One or several of these curcumins may be used as a supplement to mollify the body's normal inflammatory responses. The polyphenols trans-resveratrol and curcumin selectively bind human CB₁ cannabinoid receptors with nanomolar affinities and function as antagonists/inverse agonists. Curcumin, whose active moiety: diferuloylmethane, is a well-known polyphenol molecule that is an active taste constituent of the spice turmeric (Curcuma longa). The crude spice has been used for dietary and medicinal purposes for several centuries. Curcumin regulates various signaling molecules including several inflammatory molecules, cytokines and chemokines, adhesion molecules, transcription factors, enzymes, protein kinases, protein reductases, carrier proteins, cell survival proteins, cell-cycle regulatory proteins, drug resistance proteins, growth factors, receptors, DNA, RNA, and metal ions. These stem from curcumin's ability to bind CB₁ with nanomolar affinity and micromolar affinity with CB₂. Curcumin shares structural motifs with several cannabinoid receptor ligands.

Trans-resveratrol receptor shares characteristics with cannabinoid receptors. The affinities of trans-arachidins, trans-resveratrol, and trans-piceatannol for CB₁ and CB₂ are different in that trans-resveratrol and analogs bind CB₁, while isoprenylated trans-resveratrol derivatives tA1 and tA3 bind CB₂. The affinity of trans-resveratrol and trans-piceatannol for CB₂ is 5- to 10-fold lower than for CB₁. All compounds except tA3 exhibit ˜2-to ˜10-fold selectively for binding CB₁ relative to CB₂.

Magnolol, a biphenyl neolignan from Magnolia officinalis, magnolol acts as a partial agonist for CB₂, while honokiol is less potent but has full agonistic activity at CB₁ and antagonistic properties at CB₂. Malyngamide B binds both CB₁ and CB₂, with moderate potencies as an agonist anti-inflammatory compound. While many cannabinoids support nitric oxide (NO) production, magnolol inhibits NO production with an IC₅₀ ˜6.2 μM.

Sabinene from several diverse plants, including, but not limited to: Norway spruce, black pepper, basil, Myristica fragrans (the world's main source of nutmeg), etc. Sabinene is optically active with (+) and (−) enantiomers. Sabinene is also responsible for the unique flavor and aroma of marjoram. Sabinene is also present in other spicy spices like cardamom and cloves.

Sciadonic acid from the seeds of a coniferous plant, Sciadopitys verticillate (umbrella pine) exhibits cannabimimetic activity by increasing intracellular Ca²⁺ levels in cells expressing CB₁.

Isobutylamide and dodeca-2E,4E-dienoic acid isobutylamide, have been isolated from Echinacea purpurea and Echinacea angustifolia. Chemically, these alkylamides show structural similarity with AEA and bind CB₂ more potently than the endogenous cannabinoids with the values (CB₂ approximately 60 nM; and CB₁>1500 nM) and act as full agonist on CB₂ in the nanomolar range. Interactions of alkylamides with CB₂ is shown by immunomodulatory effects of Echinacea preparations or intact Echinacea. The Echinacea alkylamides, including, but not limited to: those above and dodeca-2E,4E,8Z,10Z-tetraenoic acid alkylamides exhibit relevant cannabinoid receptors binding affinity with a tertiary alkylamide, 1-[(2E,4E,8Z)-tetradecatrienoyl] piperidine, having the most potent binding affinity to both CB₁ and CB₂ with values of 0.8 μM and 0.16 μM, respectively. It demonstrates CB₂ selectivity with a CB₁/CB₂ affinity=˜5. Alkylamides: dodeca-2E,4E-dienoic acid isobutylamide, tetradeca-2E,4E-dienoic acid isobutylamide, tetradeca-2E,4E,8Z-trienoic acid isobutylamide, and 1-[(2E,4E,8Z)-tetradecatrienoyl] piperidine have great affinities for CB₂ and much lower affinities for opioid receptors.

β-Caryophyllene is a volatile sesquiterpene found in essential oils of several plants including, but not limited to: cloves, oregano, cinnamon, black pepper, hemp, rosemary, and hops. It is used in foods, cosmetics, and fragrances as a preservative, additive, and flavoring agent. It is approved by several food and flavor regulatory agencies including United States Food and Drug Administration (FDA) for its use as a food additive. β-caryophyllene binds CB₂. It has a peppery, woody and/or spicy smell. β-caryophyllene exerts potent cannabimimetic anti-inflammatory effects in mice.

The flax plant makes cannabidiol (CBD) and a mixture of similar compounds that have demonstrated anti-inflammatory effect. Cannabichromene (CBC) and some related compounds are found in Chinese rhododendron. Ajulemic acid is a synthesized non-psychoactive cannabinoid that has also now been found in small quantities in several plants. Ajulemic acid has demonstrated anti-allergic/anti-allergic capacities. N-Isobutylamides compounds found in the electric daisy have cannabinolic activity similar to several other cannabinolic amides (e.g., AEA) and like them has capacity to regulate pain sensation and inflammation.

Other phyto-compounds or derivatives include but are not limited to: abinene, α-pinene, 4,8-dimethyl-1,7-nonadien-4-ol, 2-hydroxy-4-methyl-valeric, acid methyl ester, octanal, O-cymene, eucalyptol, α-phellandrene, cis-sabinene, hydroxide, myrcenol, terpinen-4-ol, α-terpineol, β-thujene, ç-terpinene, trans-α-ocimene, carveol, β-citral, guanidine, geraniol, bornyl, acetate, β-pinene, thymol, geranic, acid methyl ester, α-terpinyl acetate, d-limonene, eugenol, geranyl acetate, dihydrocarvyl acetate, α-ylangene, cis-dodec-5-enal, 3-phenyl-2-propenoic, acid methyl ester, β-elemene, c, vanillin, epoxy-α-terpenyl acetate, butanoic, acid 2-methyl-, 3,7-dimethyl-2,6-octadienyl ester, 1-methyl-4-(1-acetoxy-1-methylethyl)-cyclohex-2-enol, 1,2,3,4,4a,5,6,8a-octahydro-4a,8-dimethyl-2-(1-methylethenyl)-, [2r-(2à,4aà,8aà)]-naphthalene, p-mentha-1(7),8-dien-2-ol, ç-muurolene hydroxy-α-terpenyl acetate, nerolidol, geranyl bromide, (−)-α-panasinsen, pyrocatechol, ç-elemene, 9,10-dehydro-isolongifolene, à-calacorene, cis-verbenol acetic, acid, 1-methyl-1-(4-methyl-5-oxo-cyclohex-3-enyl)ethyl ester, alloaromadendrene, z,z-2,6-dimethyl-3,5,7-octatriene-2-ol, 4-epi-cubedol, 2-oxabicyclo[2.2.2]octan-6-ol, 1,3,3-trimethyl-acetate, patchoulane, farnesol, caryophyllene oxide, cis-lanceol, ledene oxide-(ii), farnesol acetate, 6-epi-shyobunol, falcarinol, phytol, aromadendrene oxide-(2), heptacosane, longipinene, epoxide, hentriacontane, decamethyl-cyclopentasiloxane, geranyl, isobutyr, hexamethyl-cyclotrisiloxane, 1-docosene, tetratetracontane, dodecamethyl-cyclohexasiloxane, etc.

A hexane or similar extract of a cardamom, e.g., Elettaria repens, is rich in polyphenols, flavonoids, and terpenoids. The crude extract comprising one or more of these compounds can be used with cannabinolic effect or may be further fractionated for improved taste, color, smell efficacy, etc. A supplement comprising E. repens is a potent antioxidant, for macromolecules such as DNA, protein and lipid damage protective properties and also effective against carrageenan-induced acute inflammation and paw edema in rats. E. repens administration down-regulates carrageen induced increase in cytokines such as COX-2, IL-6, and TNF-α. E. repens exerts antioxidant effects by restoring SOD, catalase, GSH levels and as a result inhibits lipid peroxidation.

Cannabinoid agonists and antagonists have been synthesized and may be available for modulating the immune system through their cannabinolic activities.

Catechins generally possess moderate affinities for CB₁ with less efficacious binding to CB₂.

R-WIN 55,212-2 is a synthetic cannabinoid that strongly inhibits the interleukin-1 (IL-1) induction of the adhesion molecules intercellular adhesion molecule-1 (ICAM-1) and vascular cell adhesion molecule-1 (VCAM-1) and the chemokine IL-8.

CB₁ and CB₂ cannabinoid receptors are induced in response to hepatic injury.
Hepatic inflammation is linked to hepatic fibrosis in models of fibrogenesis. Kupffer cells are crucial and promote activation of hepatic stellate cells (HSCs). Activated T lymphocytes and neutrophils also contribute to inflammatory microenvironment leading to HSC activation and fibrogenesis. Kupffer cells are shown to express high CB₂ mRNA which is known to mediate anti-inflammatory effect by suppression of TNF-α and IFN-γ and stimulate anti-inflammatory cytokines such as IL-I0 and also inhibit macrophage migration at sites of inflammation leading to anti-fibrogenic effects of endocannabinoids in the liver. The selective CB₂ receptor agonists JWH133 and HU-308 have also been shown to decrease TNF-α, ICAM-1, and VCAM-1 expression in human liver sinusoidal endothelial cells (HLSECs) expressing CB₂ receptors and thus decreased adhesion of human neutrophils to HLSECs, thus depicting a role for CB₂ receptor in endothelial cell activation and endothelial-inflammatory cell interactions (Rajesh et al., 2008).

While synthetic cannabinoids should be used with care in frequency and volume of dosing, one characteristic of the cannabinolic systems is that they are fantastic self-regulators. For example, exogenous AEA and similar phyto-compounds that bind endogenous receptors set in motion pathways to rebalance ad restore cannabinoid metabolisms including related pathways for inducing receptors synthetic enzymes and even the degradative enzymes. Small frequent doses can be all the organism requires for superbly balanced cannabinolic controls.

Native, phytomimetic, and/or synthetic cannabinoids can be directly administered to the recipient that may benefit from cannabinolic rebalancing by any suitable means. For example, they may be delivered orally, buccally, sublingually, nasally, vaginally, rectally, transdermally, ocularly, as a vapor, etc. For example, suitable formats include but are not limited to: a buccal gel, spray, lozenge, drop capsule, paste, etc., as a nasal spray, drop, lozenge, etc. as an edible supplement or food additive, for example as drops or in pet treats or human snacks, etc., a suppository, a skin patch, eye drops, implant, etc. There is no restriction on suitable packaging. Another option involves pro-cannabinolic compounds, compounds metabolized by the organism to become cannabinoids which are also suitable as compositions for administering or delivering the active substance.

Both ω-6 and ω-3 Essential Fatty Acids (EFAs) are two groups of poly-unsaturated fatty acids (PUFAs) required for the production of endocannabinoids and cannabinoid receptors. Mammals must consume both types of these EFAs because the body cannot synthesize them in amounts required for good health. ω-3 PUFAs are generally anti-inflammatory, while ω-6 PUFAs are often proinflammatory. Given that ω-3 acids and endocannabinoids share similar benefits, they may often be combined in a supplemental composition for use n accordance with this invention.

A deficiency in ω-3 PUFA causes CB₁ to uncouple from its effector G proteins, essentially disabling them. ω-3 consumption upregulates CB₁ and CB₂ expression, along with increased levels of endocannabinoid synthesis enzymes. Foods including, but not limited to: flax, hemp, and chia seeds, along with walnuts, are excellent vegetarian sources of ω-3PUFAs. The ω-3 PUFA in these foods is α-linolenic acid, which is converted into the more synthetically useful longer-chain ω-3 PUFA compounds eicosapentaenoic acid (EPA) and docosahexaenoic acid (DHA) to synthesize many other compounds.

ω-3 PUFAs include but are not limited to: hexadecatrienoic acid (HTA), α-linolenic acid (ALA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), clupanodonic acid, docosahexaenoic acid (DHA), tetracosapentaenoic acid, tetracosahexaenoic acid (nisinic acid), etc.

Haplosamate derivatives are the first naturally derived cannabinomimetic compound belonging to steroid family. They represent another new chemical class of cannabinoid receptor ligands. This group of steroids includes but is not limited to: haplosamate A and haplosamate B.

Haplosamate A and desulfohaplosamate have opposite effects. Haplosamate A has strong affinity for CB₁. Desulfohaplosamate has higher affinity for CB₂. The 7-monoacetylated derivative of haplosamate A exhibits affinity to both CB₁ and CB₂ cannabinoid receptors in comparison to its parent compound. However, acetylation at C-4 or dialdehyde derivative results in the loss of affinity on both CB₁ and CB₂.

Euphol has potent immunomodulation and anti-inflammation effects. CB₂ plays a predominant role in this. The antihyperalgesic effect of euphol is similar to the effects caused by ACEA, a CB₁ agonist, and JWH-133, a CB₂ agonist.

18β-glycyrrhetinic acid and its diastereomer 18α-GA are triterpenoid saponins from the roots of Glycyrrhiza glabra L., popularly known as “licorice”. They are generally used as natural sweeteners and flavoring additives in food and have been found in traditional medicines purported to possess antimicrobial, anticancer, and anti-inflammatory properties. The inhibitory activities of licorice extract i involve a dose-dependent decrease in intracellular Ca²⁺ levels.

Aliquirtin, glabridin, and 18α-glycyrrhetinic acid also exhibit inhibitory activity against Ca²⁺ flux induced by AEA, whereas 18β-glycyrrhetinic acid has stronger potency evidenced by more than 90% inhibition in responses to CB₁ agonist.

The oxylipin, 3-hydroxyarachidonic acid (3(R)-HETE), is an intermediate of the β-oxidation of arachidonic acid and has an important role in the life cycle of fungi. T

Clostridia affect the gut: Acting through several immune cells and cytokines, the bacteria can prevent peanut proteins that can cause allergic reactions from gaining access to the bloodstream. Clostridia, a gut bacterium, in some cases protects against food allergies. By inducing immune responses that prevent food allergens from entering the bloodstream, Clostridia minimizes allergen exposure and prevents sensitization—a key step in the development of food allergies. Genetic analysis reveals that Clostridia causes immune cells to produce high levels of interleukin-22 (IL-22), a signaling molecule known to decrease the permeability of the intestinal lining. Cannabinoids may be supplemented to aid this effect.

Mammals have their own endocannabinoids. But many cannabinoid substances, the phytocannabinoids, are found in plants. Cannabinoid substances are found throughout the plant and animal kingdoms. Cannabinoid receptors are ubiquitously present in most eukaryotes, being present in chloroplast membranes of plants and in nuclear and mitochondrial membranes of other eukaryotes.

CB₁ and TRPV1 are active in mitochondrial membranes where the cannabinolic pathways act to balance distribution and activities of mitochondrial proteins and mitochondrial metabolism itself. In severe cases, the cannabinoid receptors in the nuclear membrane and in mitochondrial membranes are important mediators in apoptotic cell death.

Another class of compounds with wide systemic metabolic effects comprises corticosteroids or glucocorticoids. While immune system imbalances can occur throughout an organism's lifespan, corticosteroid pathways tend to decline in efficiency as animals age and their mitochondria become burdened by previous metabolic compromises that accumulate over time.

One additional important factor involved in the present invention is that as the animals age androgenic influences of endogenous hormone, especially testosterone, continuously wanes. While high prolonged supplementation with this class of steroids suffers from serious side-effects, these steroids are natural compounds and when present in appropriate concentrations can augment other mechanisms for optimizing healthful living.

In the blood serum of mammals, testosterone exists primarily bound to a protein, typically albumin or sex hormone binding protein. Unbound testosterone is referred to as “free testosterone”. The term “total testosterone” refers to the total amount of testosterone in the blood serum, that is, the combined amount of protein-bound testosterone and free testosterone. The typical half-life of “testosterone” in the blood serum ranges from 10 to 100 minutes. While many of these steroids and steroid mimetics are not presently approved for human as therapeutic compounds, several may be made available in the form of supplements to humans and other mammals.

Testosterone, a primary anabolic steroid, is metabolized into dihydrotestosterone in the body by way of the 5-alpha reductase (5AR) enzyme (this means that dihydrotestosterone is a metabolite of testosterone), and furthermore, nandrolone is a byproduct of the aromatization (conversion) of testosterone into estrogen. With this knowledge, it then stands to reason that testosterone quite literally is the origin of all anabolic steroids. Without testosterone, DHT and nandrolone would not even exist, and therefore without the existence of DHT and nandrolone, their individual derivatives and analogues would also not exist.

Testosterone itself is the principal male sex hormone. Hormones are defined and classified as chemical messengers of the human body, which means that hormones are what carry messages to different cells and tissues in the body to tell those cells and tissues what to do (grow muscle tissue, heal and repair, manufacture important components, perform a specific job, etc.). Without hormones of all different types, all functions within the human body will proceed unregulated and out of control. How much testosterone the average male produces is dependent on many different factors, which include: individual genetics, age, lifestyle habits, nutritional habits, and activity levels. On average, it has been determined that the median level of testosterone production among 30 year old human ˜150-175 lb. males is between 50-70 mg weekly. Where any given individual might land within that range is dependent on the aforementioned factors. It is common knowledge that the most prominent effects of the hormone testosterone appear and are experienced during puberty, which is evidenced by an increase in testosterone production and secretion, and will typically reach the highest endogenous levels at this point in any given man's life. This significant increase in testosterone serves to impart very important physiological changes of the male body. Testosterone governs many different functions within the body. The nature of hormones in the circulation is to govern systemic functions remotely around the body, and testosterone is no exception to this. Dosing can be based on the blood levels of the intended recipient, their weight, the chemical nature of the supplement (bioavailability, half-life, partitioning in body compartments, binding to proteins, etc.) to maintain the animal at an androgen level approximating or preferably exceeding by about 1.5, 2, 2.5 or 3 times the previous average levels.

Androgens such as testosterone and DHT bind to androgen receptors (ARs) in cells. The resulting androgen-receptor complex regulates gonadotropin secretion and spermatogenesis. The androgen-receptor complex is responsible also for external vinilization and for most androgen actions during sexual maturation and adult life. DHT is an especially potent androgen because it binds with greater affinity to androgen receptors than testosterone does. Testosterone production in intact mammals is stimulated by luteinizing hormone (LH). It is understood that follicle stimulating hormone (FSH) stimulates testosterone production also. Testosterone concentrations in the blood serum are regulated in part by a negative-feedback pathway in which testosterone inhibits the formation and/or secretion of luteinizing hormone-releasing hormone (LHRH). LHRH acts to stimulate secretion of LH by the pituitary gland. Testosterone acts also by regulating the sensitivity of the pituitary gland to LHRH.

Taking dogs, aka canines, as an example, the present invention provides improved health and longevity for the animal component of the relationships humans are finding more and more significant. Maintaining a pet's well-being can support oxytocin levels in a human owner. Oxytocin is one of the natural inducers of AEA synthetic pathways. Thus, improving pet's activities that lead to oxytocin release in the human owner's body can have a beneficial side-effect of supplementing cannabinoid production and circulating levels in the human owner(s).

On top of this, animals exhibit large differences in their mitochondria and mitochondrial activities. By optimizing energy metabolism through patient specific monitoring and improvement in mitochondrial metabolism, the vitality effects observed through androgen balancing may be further enhanced. Accordingly, the invention preferably includes as alternative approaches wherein in conjunction with androgen balancing as an anti-aging measure that optimizes activities and/or in conjunction with system rebalancing by enhancing cannabinoid activities, additional improvement may be obtained by also optimizing mitochondrial activity, metabolism and performance.

Common diseases in the aging dog include: arthritis, which reduces activity levels and may make the animal more irritable or reclusive; obesity, which can acerbate arthritis and other diseases such as cardiomyopathy and usually reduces animal activity levels; joint dysplasia, which reduces animal comfort and activity; gum disease; diabetes; blindness of various etiologies; dementia; and other diseases of aging familiar in humans. Metabolic functions may be impaired, for example, adipose tissue may experience accelerated or location improper deposition and/or aberrant utilization, glucose metabolism and metabolism of other sugars may be altered though diabetic effects and compensating metabolic shifts.

For example, in humans both free testosterone and total testosterone have been documented in their decline as a male ages. Up to their fifties, human males essentially maintain total testosterone with about a 25% drop in free testosterone from about the age of thirty to about fifty. In the ensuing years both total and free testosterone continue to decline until about the age of eighty the levels are only about half the levels previous to age fifty. While there are differences in effects of aging between mammals, in general testosterone levels decline with age as the animal's vitality also declines. As an approximation, 25% supplementation may be used as augmentation beginning at approximately age 5 in dogs and age 7 in cats. The supplemented amounts will depend on the androgen compound chosen and its comparison in activity to testosterone. Advisedly testosterone levels will continue to be monitored with additional supplementation as Testosterone activity lessens.

Evidence is building that age related reduced testosterone levels in human males, and thus in other mammals, may be related to growth of pot bellies and possibly, heart attacks, strokes, osteoporosis, clinical depression and some presentations of Alzheimer's disease.

A body of evidence relating to human females suggests that testosterone levels may be important factors with regard to depression, activity level and general sense of well-being. There is also evidence that in human females a testosterone supplement may improve activity levels and maintain a leaner body. Low testosterone levels in human females have been associated with lack of motivation and a sense of fatigue. The common weight gain and increased adipose tissue deposition in women starting approximately 10 years prior to menopause coincides with a commonly observed decreased level of circulating testosterone. This suggests an important component of the present invention relating to maintaining testosterone balance will benefit both male and female mammals, including, but not limited to: canines, equines and felines.

Importance of Balancing (Controlling) Androgens in Circulation

Although it is reported that thousands of androgenic hormones have been made, for example to achieve androgenic effect but to avoid detection by sports monitoring organizations, “testosterone” is used as an example throughout this discussion since testosterone is an inexpensive and commonly reported androgen that has been used and abused by male and female humans. As testosterone replacement or testosterone level augmentation raises testosterone levels up to or possibly slightly exceeding previous normal range it has a high success rate of alleviating many conditions associated with aging that have been found to compromise human and animal health and general well-being. Testosterone and analogues have successfully treated or managed female breast cancer, hereditary angioedema, anemia, multiple muscle wasting diseases including HIV/AIDS, severe burns, acute and chronic wounds, general caloric wasting, muscular atrophy, osteoporosis, male infertility, adolescent growth failure, osteoporosis, female libido problems, Turner and Klinefelter Syndrome, menopause, chronic dysfunctional uterine bleeding (menorrhagia), endometriosis, and many others. Recent reports suggest that testosterone has an import effect in strengthening connective tissues such as ligaments, for example the anterior cruciate ligament (ACL) in the knee.

Actual ACL strength has been measured in animals with a conclusion that exposure to the androgenic hormone somehow makes the tissue less prone to rupture during stretching. Supporting this testosterone effect on at least the ACL is the perplexing high incidence of ACL tears in girl's knees well in excess of their participation in stressful sports. These vastly different effects associated with testosterone in both males and females suggest that an appropriate amount of circulating androgen is important for general health and well-being.

Thus, these and other findings suggest that maintaining an optimized testosterone level as the animal ages can result in improved vigor, reduced injury, and greater activity and possibilities for social interaction. Management of circulating testosterone also has the possible effect of preventing or reducing injury, such as muscle or joint injury, and can thereby appear as an anti-aging agent to maintain a higher level of activity available to the animal and to the animal's human companion(s).

While testosterone level management and supplementation where warranted have proved successful in improving specific health effects all over the body, administration has been carefully controlled within the medical community to avoid misuse and deleterious effects that can be associated with elevated testosterone levels that exceed safety limits.

Popular acceptance of testosterone balance in the body may suffer from reports of overuse and abuse of supplements in human males who may have obtained testosterone for unapproved and especially for ineffective treatments. For example, the growth of novel disease states such as erectile dysfunction (ED), the expense of prescribable ED pharmaceuticals, and claims on the Internet touting androgenic compounds such as testosterone and testosterone mimetics as effective treatments coincides with a 2000% increase in testosterone sales happening in the United States from about 1990 to about 2002. It is also widely accepted that testosterone, and the then more difficult to detect design androgenic compounds, were used in Olympic and other high level sporting competitions because of the desirable effects on physical performance. In the more general population several occupations have been prone to testosterone overuse and abuse. For example, police officers, security guards and bouncers, having become aware of testosterone's profound efficacy have made use of androgens to improve at least their perceived on-job performance and general performance in life situations. Such stories may interfere with easy availability of supplemental steroid compounds for use in animals. In some instances a veterinarian prescription may be necessary to obtain the compound as a supplement.

Proper Androgen Levels Can have Anti-Aging Effect, but Misuse is Dangerous

The observation that as testosterone levels decrease during aging and that testosterone is believed to enhance muscle development suggests that the occurrences are not purely coincidence. In fact, muscle tissue expresses androgen receptor (AR) protein so it would be understood that testosterone would influence muscle metabolism. Supporting evidence that testosterone supplementation or replacement increases muscle fiber protein synthesis and that pluripotent stem cells capable of differentiating into muscle fiber cells have high levels of AR expression suggests a causal relationship exists. Accordingly, Supplementing androgen, e.g., testosterone to an aging animal may maintain or build muscle mass. Better muscle mass is associated with lower incidences of diabetes so the benefits of testosterone balancing would be expected to cascade through many organs and tissues. But at lower levels of activity while testosterone balance may produce profound benefits an even greater improvement is possible if mitochondrial optimization is partnered with the balancing. Not intending to disparage the invention approach wherein animal health and relationships are augmented by testosterone balancing, the invention recognizes that the augmentation can be amplified by 1) maintaining or optimizing mitochondrial performance as part of the intervention; and/or 2) by taking advantage of cannabinoid compounds to facilitate balancing throughout additional systems. On the other hand, allowing mitochondrial impairment or other systemic imbalances to degrade benefits of androgen balance would be seen as slowing or limiting benefits of the balancing itself. In fact, relating to aging, some believe that oxidative stress of mitochondria may have a major role in age related energy deficit.

Androgenic compound abusers have contributed to testosterone's and other androgenic hormones' shocking disparagement in general news media through reports of sometimes violent activities and severe health outcomes such as brain tumor, but perhaps partly related to publicity from these mainstream press warnings about androgenic use and “over manliness”, abuse continues in a significant segment of the population. Though serious abuse may present long term problems for the individual and society, society in general can understand that the abuse is a result of testosterone's positive effects.

While some desired effects, for example, increased muscle mass in body builders and other professional athletes, may be valued for their immediate effects, long term effects, for example use over decades, has been shown to increase propensity for heart attack and stroke. The length of administration and the expected remaining lifespan of the individual should be considered before enhancing androgen in the bloodstream.

Other noted effects include elevated LDL and higher LDL/HDL ratio, increased blood pressure, increased cancer, for example, brain and liver, and difficulty in movements that may be caused by excess tissue deposition. Androgen abuse is also associated with testicular wasting or atrophy which in humans may or may not be desired depending on one's desires for fatherhood. In neutered dogs, this of course would not be a relevant concern.

Most of these recognized problems can be avoided or minimized simply by managing testosterone blood levels to levels more prevalent in normal animals or limiting administration to older animals with for example an expected remaining lifespan of about a decade or less. For example, the irritability, aggressive behaviors, rage, violence, delusions, manic eating, etc., appear to be associated with abuse of androgens that involves massive dosing regimens; liver disease and tumor events appear associated with long term administration of moderate to high doses.

The present invention seeks to avoid these problems by optionally providing systems wide rebalancing tools in the form of cannabinolic augmentator supplements or facilitators and by monitoring, either through behavioral observation, or more preferably by measuring blood, saliva, urine, or skin androgen or androgenic activity. Even minor undesired elevation above a targeted amount, perhaps resulting from metabolic differences, change in food, or a mistake in dosing or formulation can be properly corrected. Long term effects seen over decades may not be a problem at all in shorter lived species. But age may be considered as a factor.

Proper Androgen Balance is not for Amateurs

The natural testosterone molecule is poorly available to a mammal's circulation when it is delivered by oral administration and absorbed across the intestinal wall into the hepatic portal system. Liver metabolizes most ingested testosterone before it can enter general circulation. An attempt to surmount this obstacle by alkylating (methylating) the C-17α position of the molecule has been essentially abandoned for human use because of toxic effects on the liver. Testosterone undecanoate is not modified at the C-17α position and appears to avoid this toxicity issue perhaps because its absorption is not through the portal system.

Due to the efficiency of the mammalian digestive system in breaking down foodstuffs to simpler molecules delivering large amounts of testosterone through the gastro-intestinal track, including the hepatic portal system most, but not 100% of an intact lipid like the fat-soluble hormones will reach general circulation. However, since the digestive process is not 100% efficient (a small percentage of large molecules will bypass or escape the degradative process) after giving a large dose a small but effective amount of delivered testosterone will enter general circulation. Since digestive efficiencies vary greatly between individuals, frequent monitoring of circulating levels to maintain proper balance is highly recommended.

To avoid the chemical breakdown and filtering pathways that have evolved for digesting natural environmental feeds and possible toxins, testosterone can be bound, packaged or complexed in a less natural manner to enhance absorption of the active molecule, through a non-portal pathway, for example, through the lymph system. Complexing testosterone with a form of sex hormone binding globulin (SHBG), which carries the hydrophobic (lipid based) hormone in the circulatory system may be one important means for delivering testosterone through the general circulation. Liposomes and other hydrophobic carrier systems are possible delivery agents. A coating or packaging that protects the testosterone carrier from gastric acid and enzymes may be designed for removal in the small intestine to permit testosterone to be carried into the lymph system.

Another means for avoiding hepatic-portal absorption is have the hormone absorbed into the circulation before it encounters the portal pathway. Sublingual or buccal delivery would accomplish this feat. Most dog owners would doubt that their pet would retain a pellet o similar item under its tongue. However, extensive or prolonged exposure to the mouth endothelial tissues for uptake very early in the GI track, well before action by stomach acid or possible sequestering into the portal system, is easily accomplished by exploiting the chewing behavior of canines. Dogs, for example, will spend unbelievable time and effort chewing raw hide or scented or flavored dog toys. Thus, one composition that might be used in practicing the present invention is a hormonal compound complexed or packaged in a chew toy. Incorporating testosterone in a chew toy which is facilitated by testosterone's lipophilicity is an effective means for effecting prolonged exposure through the gums and other early GI tissues. Direct supplementation using an androgen or testosterone molecule in food is not without its downsides. Testosterone in its native state or in chemically modified form has positive and negative consequences from use. Accordingly, use of the compositions is preferably in conjunction with repeated monitoring of androgen, e.g., testosterone, balance to minimize or avoid undesired effects while benefiting from the desired effects.

Circulating testosterone may be increased to a degree using compounds such as vitamins to stimulate production or to inhibit breakdown. For example, vitamin D, has been shown to protect the liver from viral and chemical toxicity. Accordingly, vitamin may be co-administered with an androgen to help protect the liver from toxic response to the androgen. Vitamin D has a second beneficial attribute in that it is lipophilic as are the cholesterol derived sex hormones such as androgens like testosterone.

Vitamin D can serve as a lipid carrier for other lipid molecules such as testosterone. When co-administered with testosterone Vitamin D both serves as a delivery vehicle until the SHBG and testosterone are able to associate. The testosterone androgen is circulating to find its target(s) and the vitamin D can migrate to the liver to exert its protective effect there. Other lipid vitamins, such as vitamin A, vitamin E and/or vitamin K may also serve as lipid carriers to hold lipophilic androgen in circulation until it is able to be gathered by circulating SBHG.

So in addition to oral supplementation with androgen, such as testosterone, testosterone balance can benefit from the presence of lipid vitamins and from inducing expression of SBHG using one or more means known in the art.

Sub-dermal identity chips are popular with pet owners in the United States and are growing in popularity worldwide. This illustrates that pet owners are receptive to “foreign” bodies being inserted into their animals. Similarly, several forms of female birth control in humans make use of an implanted rod or stick that slowly dispenses a sex hormone. This acceptance suggests that many pet owners may welcome androgen supplementation in this manner to avoid risks, for example of hepatic toxicity. Such device implanted in an animal might use any of the available delivery options. Shorter lasting implant (several months) might use an osmotic pumping mechanism. Another form of implant might use controlled solubility or diffusion where the androgen is part of a slowly dissolving matrix or is incorporated within a barrier that controls androgen diffusion into the bloodstream. Such implanted devices will help ensure better hormonal balancing because of the required visits to the veterinary clinic where a technician or veterinarian can assay circulating androgen and refresh the implant.

These extensive effects of the anabolic steroids, though beneficial in large often result in well-known side effects, some of which are mentioned above. When administering anabolic steroids or other metabolism altering compounds, systems and subsystems and their cell suffer imbalances. Coordinating such anabolic therapies with a supplemented cannabinoid therapy can maximize benefits with reduced side effect issues.

Caution is Warranted Because of Testosterone's Wide Effects, but Benefits are Immense

Testosterone and other androgenic compounds have historically been misused or abused because of their profound effects in multiple locations throughout the body, especially in muscles and sex organs.

Testosterone activity is mediated trough androgen receptors that are found in a multitude of tissues throughout the body. To function the testosterone molecule crosses the cell membrane and binds to an intracellular receptor (AR) present in the cytosol of many cells. The testosterone-receptor complex then migrates into the nucleus where it can bind specific deoxyribonucleic acid (DNA) segments to control gene expression by activating synthesis of specific messenger ribonucleic acid (mRNA) segment molecules to increase transcription (copying the targeted DNA) and processing the copy to inaugurate protein synthesis controlled by the targeted segment of DNA; which, for example, in muscle cells, may increase production of the proteins actin and myosin.

After this transcription/translation process is complete, the testosterone-receptor complex dissociates and the receptor is recycled along with the hormone to repeat this process multiple times. Androgenic receptors, including some responsive to testosterone metabolic products or metabolites such as dihydroxytestosterone (DHT) have been observed throughout the body in various tissues including, but not limited to: skin, scalp, prostate, thyroid, muscle fiber cells, muscle stem cells, pancreas, bladder, bone marrow, stromal cells, endothelial cells, macrophages, myeloblasts, myelocytes, neutrophils, megakaryocytes, corneal cells, lens cells, iris cells, cilliary body cells, adrenal cells and adipose (fat) cells. A casual observer should understand that testosterone activity would in all likelihood be relevant on many organs and tissues throughout the body.

Accordingly, the present invention recognizes that decreased testosterone levels such as those occurring during the aging process may result in diminished or suboptimal function of at least one and probably many organ systems in the body. As a result controlled restoration of testosterone presence and activity can have profound beneficial effect with regard to multiple physiologic functions. Improving at least one of these functions, and preferably several can result in a general improvement in the animal's physiology and well-being. Enhancing mitochondrial metabolism and/or providing cannabinolic active or activating compounds can support the androgen augmentation effects.

Administration of testosterone to restore normal physiological levels can help to restore to a more youthful state and improve the function of many of the different systems where testosterone's effects on the cellular level are accomplished. This includes, for example, action in the bone marrow that increases red blood cell count, which translates to increased endurance, improvement in energy, well-being, and restoration of muscle mass.

There are various studies that have determined where, on average, testosterone levels should be in males according to various age groups. Generally, testosterone in human males declines about 1% per year from the late thirties. For animals, the decline may be steeper depending on size, species and lifespan, and will generally occur at a younger but still at a middle age.

Osteoarthritis and hip dysplasia are especially common and problematic in larger dog breeds and larger dogs in general. Dogs will reduce activity level and avoid some previous activities to hide the symptoms or to avoid associated pain. Aging is also associated with a general lethargy that can be a result of or mask other diseases such as a failing heart, painful joints, decreased muscle tone, arthritis, etc. and may be a factor in weight gain that can cause or exacerbate other disorders. Increased dysplasia and obesity have been observed to have increased occurrence in canines that have been spayed or neutered. While the benefits, in most cases, necessity of spaying or neutering are profound, the procedure does remove a major source of androgenic hormone, testosterone and its metabolites, from the animal's physiology. While other organs such as the adrenal produce androgens, often the amount decreases as the animal ages and becomes insufficient for optimizing animal activity and health. Similar concerns prevail in cats, but may be less noticed. Vigilance is advised.

While larger dogs appear more prone to hip dysplasia, the outcome is observed in smaller dogs also. Orthopedic Foundation for Animals reports that the top 20 breeds exhibiting dysplasia were: bulldog, pug, dogue de bordeaux, neapolitan mastiff, otterhound, St. Bernard, boerboel dumber spaniel, black Russian terrier, Sussex spaniel, cane corso, basset hound, fila brasileiro, Argentine, dogo, perro de presa canario, American bulldog, Norfolk terrier, Maine coon cat, boykin spaniel, and French bulldog. Clearly this phenomenon is a concern for small as well as large canines. This is not as frequent a concern in cats, but the generally teachings are still applicable.

Testosterone is the main male sex hormone, predominantly synthesized in the testes by Leydig cells (95%). In human males, for example, a small amount of testosterone is produced by adrenals (5%). Classic effects of testosterone include the androgen effects supporting-the growth and development of sex organs, the formation of stereotypical dominant male behavior (aggressive, attack behavior, territorial or harem protection, and other undesired behaviors), anabolic functions (maintaining muscle mass including myocardiocytes), stimulation of the synthesis of organ (specific proteins in kidneys, liver, sebaceous and sweat glands in animals that have them, maintaining bone density, hematopoiesis (stimulation of erythropoietin generation in kidney and stimulation of erythropoiesis in the bone marrow).

Androgens include, for example, 17β-hydroxyandrost-4-en-3-one, commonly known as testosterone, and dihydrotestosterone (DHT), a metabolite of testosterone. Testosterone is a naturally occurring androgen which is secreted in males and, to a much lesser extent, in females. In males, testosterone and DHT are responsible for normal growth and development of the male sex organs and for maintenance of secondary sex characteristics. In females, testosterone and DHT are believed to be important for normal growth, sexual desire, and sexual function. In addition, androgens promote retention of nitrogen, sodium, potassium, and phosphorus, and decrease the urinary excretion of calcium. Androgens have been reported to also generally increase protein anabolism, decrease protein catabolism, and stimulate the production of red blood cells.

Common symptoms of androgen deficiency that may be of specific concern to pet owners are blood pressure fluctuations, cardialgia, psycho-emotional disorders (irritability, decreased overall health and performance), decreased memory and attention, insomnia, depression, somatic disorders (reduction of muscle mass and strength, increase of adipose tissue amount, bone loss, visceral obesity and thinning of skin. Several studies have shown that a decrease of testosterone concentration results in increased deposition of fat cells in various locations resulting in degeneration of the smooth muscle cells. While most research in this area has been reported with human males as target test subjects, effects are widespread and affect mammals in general and both male and female individuals.

Most pets and farm and companion animals are spayed or neutered as population control and to avoid undesired behaviors associated with the hormones that drive or control sexual activity, including mate attraction, and other undesired behaviors.

The benefits are deemed to vastly overcompensate for the changes associated with the spaying or neutering. For example, very few farmers maintain a bull; they are dangerous and difficult to control; and especially for dairy herds, artificial insemination produces more reliable timing and product. In pets, male dogs and cats become easier to manage and less aggressive, more homebound, less prone to testicular cancer; females are less prone to breast cancer, will not have repeated heat cycles attracting nuisance male callers and more frequent urination, even indoors, associated with heat to signal receptivity for males. Neutered males are less likely to wander in search of females and less likely to mark territory around and within your house.

Controlling or Balancing Levels of Circulating Androgen to Optimize Long-Term Health

A tradeoff that humans have accepted is that as spayed or neutered animals age, androgenic hormonal support such as provided by testosterone drops off. [Even in intact male and female animals androgenic support declines with aging.] While in male animals the testes would produce the predominant share of androgenic hormone, the feedback mechanisms within the body of a younger animal compensate quite adequately to maintain a general state of health.

But as the creatures age, testosterone activity falls off regardless of the animal's sex or gonadal status. This trend is small but noticeable and has generally been accepted as a part of the aging process.

While the declines in animal health are expected and accepted as part of a normal aging process, the diminished performance, comfort and health of the animals can be slowed by persons practicing the present invention.

In regular practice of medicine, often medical intervention is compromised because a patient's metabolism is weakened because of intrinsic or extrinsic factors or because the therapy itself will change normal cell metabolism. Recognizing the wide distribution of androgen receptors throughout the body multiple effects are expected in many different organs and tissues. Accordingly, the full beneficial effect of the medical therapy is limited by one or more other cell function, in particular cell energy metabolism.

Optimizing Energy Metabolism in Conjunction with Androgen Balance Promises Even More Superior Anti-Aging Benefits

Mammalian cells are eukaryotic cells and therefore, like eukaryotic cells generally, they rely on their mitochondria to produce adenosine triphosphate (ATP). In each mitochondrion at the mitochondrial inner membrane, electrons from NADH and succinate are transported by the Electron Transport Chain (ETC) to oxygen, which, when it accepts the electrons, is reduced to combine with hydrogen to make water. Along the way the ETC comprises several donor and receptor enzymes in series, eventually depositing the electrons with an oxygen. Passing electrons from donor to acceptor releases energy in the form of a proton (H⁺) across the mitochondrial membrane. This ion flux has the potential to do work. This metabolic process is known as oxidative phosphorylation and results in production of adenosine triphosphate, aka, ATP. The mitochondrion is important to cell metabolism and survival. Detailed descriptions are known or can be found in the art.

Thus, the mitochondrion organelle is essential for healthy cells and therefore for healthy animal life. ATP, an essential molecule for energy metabolism within the cell is primarily generated by mitochondria. Processes such as adaptive thermogenesis, ion homeostasis, immune responses, production of reactive oxygen species, and programmed cell death (apoptosis) are some of the more complex processes that also require appropriate ATP synthesis and transport. Mitochondria contain their own DNA (mtDNA), which serves as a template for 13 mitochondrial proteins, 2 ribosomal RNAs (rRNAs), and 22 transfer RNAs (tRNAs). However, the mitochondrion cannot function as a distinct and independent organelle. Replication, transcription, translation, and repair of mtDNA require proteins encoded by nuclear DNA (nDNA) of the hosting cell. When the host cell is sub-optimal, perhaps from androgen shortage, mitochondrial metabolism would be expected to be compromised.

Modern mitochondria have many similarities to some modern prokaryotes, even though they have diverged significantly from the early prokaryotes since the ancient symbiotic event. For example, the inner mitochondrial membrane contains electron transport proteins like the plasma membrane of prokaryotes, and mitochondria also have their own prokaryote-like circular genome. But one difference is that these organelles are thought to have “lost” most of the genes once carried by their prokaryotic ancestor. Although present-day mitochondria do synthesize a few of their own proteins, a vast majority of the proteins they require to maintain the host cell are now encoded in the nuclear genome of the host.

While androgen balance holds promise for vast improvement in animal liveliness, an additional manipulation wherein after or during androgen balancing mitochondrial function is also improved holds further promise for optimizing animal general liveliness and more fulfilling interaction with others.

Thus, the present invention relates an orally ingestible, animal life enhancing product to be administered by a human to an animal to optimize human animal relationships, and/or the animal's comfort, longevity and/or quality of life.

Importance of Hormonal Influence to Balance Physiology in Aging Mammals

Though there is variance among breeds of dogs, in general diminished androgenic influences become apparent between four and eight years of age. Some effects are seen in larger dogs at earlier ages. Some early effects, such as the puppy wildness, which though cute often invokes glee in humans as they pass and the animal becomes more predictable. These may be related to androgenic stimulants and it will be discretionary whether to begin treating these dogs at this early period or to begin treatment at a stage where animal comfort may be a larger factor. Cats, though not as variable in size, have similar concerns.

Mammalian bodies, like those of dogs, cats, rabbits, etc., have internal means of messaging. Blood flow can be increased or decreased to an area or organ. Nerves sense what is happening at different locations within the body and then transmit information to the central nervous system where multiple inputs are analyzed and coordinated to initiate an output. The output could be neurotransmitter secretion causing a nerve impulse sending instructions to another location in the body. Another very important means of internal commutation is the endocrine system which uses hormones as signaling agents. Hormones are chemicals just as neurotransmitters, but hormones have effect distant from the place of release.

Hormones are chemical messengers used to transfer information through the bloodstream from one part of the body, generally an endocrine gland, to the body in general or to a specific target organ that has a receptor capable of binding or receiving the hormone. Target organs have specialized receptors that gather information that has been transferred from the circulatory system by hormones. An example of a target organ is the uterus, which is stimulated by the circulating hormone estrogen to develop uterine glands. Hormone production—for example, testosterone, estrogen, and progesterone—is regulated by another hormone secreting endocrine gland, the pituitary, at the base of the brain.

Prohormones are building block chemicals used to produce the hormone. In general, the blood levels of sex steroid prohormones are not regulated by any substance. Instead, prohormones are generally available to assist in the production of hormones, which then act as chemical messengers to other target organs. These prohormones are essential building blocks for production of the respective hormones and may themselves be at levels insufficient for optimal hormone production. Providing functioning hormone eliminates the problem that may stem from insufficient prohormone. In some hormonal pathways, a hormone may be metabolized to an inactive prohormonal state that may be recruited when needed to produce active hormone.

The invention has a goal of administering to the animal a non-toxic, effective amount of a composition comprising an androgenic hormone such as testosterone, or a pharmaceutically acceptable salt or metabolite thereof. Hormone level or activity following administration of the composition to the animal is evaluated. The active ingredient may be bound to complexed with or incorporated in a carrier to facilitate administration and possibly to control bioavailability. The composition is re-administered to the subject as needed to maintain a desired level of circulating hormone or observed activities.

Co-administering such hormone with cannabinoid active substance to assist the body in rebalancing the hormone effects is an especially favored aspect of practicing this invention.

Definitions

In general words in this description will have a meaning as used in American English. The following list is provided as additional guidance.

ADP—adenosine diphosphate. Higher ADP levels are often associated with higher respiratory activity.

ATP—adenosine triphosphate, a primary molecule involved in energy storage, transport and release.

Biogenesis—a synthetic process occurring as part of metabolism in a living organism.

Cellular metabolism—set of chemical reactions that occurs in living organisms to maintain life. Metabolism includes both anabolism and catabolism as well as multiple pathways that maintain life functions within a cell or organism. There is no real count of an actual number of metabolic pathways. With branches and cycles within major pathways and pathways sometimes only active in specific cell types and sometimes only at select times, counting an actual number would be arbitrary. However, the skilled artisan appreciates that the total number of pathways, including subpaths numbers in the thousands. The Internet is an available resource to study classes of pathways or individual pathways. See e.g., www.itsokaytobesmart.com, though there are many web pages available relating to metabolic pathways.

Clinical improvement—An observable improvement in at least one factor in a pa quality of life.

Coenzyme Q(CoQ₁₀)—aka: ubiquinone or ubidecarenone. An oil-soluble, vitamin-like substance is present in mitochondria. CoQ₁₀ is part of the electron transport chain participating in aerobic cellular respiration to form ATP. CoQ₁₀ is especially significant because of its respiratory functions and because cholesterol inhibitors, such as statins can also inhibit synthesis of CoQ₁₀ precursors.

Desmin—An intermediate filament (IF) protein expressed in striated and smooth muscle tissues and is one of the earliest known muscle-specific genes to be expressed during cardiac and skeletal muscle development. Desmin is seen as controlling mitochondrial function by interaction with myofibrils and interacting with the cytoskeleton to affect positioning within a cell.

ETC—electron transport chain which is used to harvest energy for use in metabolism.

Kcnq2—a member of the kcnq family of proteins which act as ion channels controlling potassium (K) flux across membranes. Potassium gradients can control electrical potential across a membrane and therefore can be involved with electrical signaling within and between cells. A potassium gradient can also control flux of other ions.

kif5b and kif5b—a gene encoding the protein and the encoded a heavy chain portion of kif5 protein working through microtubules to effect appropriate distribution of mitochondria within a cell. Mitochondria are not its only cargo; the protein is also associated with lysozyme and endocytic vessel distribution and is an essential component for distribution of many proteins within a cell. Neurons also express related proteins encoded by kif5a and kif5c.

Mitochondrial integrity—Mitochondrial integrity is known as a controlling factor in apoptosis, cell controlled self-destruction. Integrity may be comprised by events including, but not limited to: membrane permeability changes, altered exposure of membrane proteins, changed expression of the mitochondrial genome.

Mitochondrial protein—a protein encoded by or used within a mitochondrion.

Mitochondrial supportive substance—a chemical that changes mitochondrial activity to benefit at least one aspect of cellular metabolism.

mtDNA—double-stranded DNA found exclusively in mitochondria that in most eukaryotes is a circular molecule. A single mitochondrion may include multiple copies of this circular mtDNA molecule.

Optimization—As used herein, optimization has the general meaning of a process leading to an improved outcome. Optimization will generally incorporate at least one facet of enhancement of number, outcome function or the like. In some uses optimization may refer to maximizing a component or process or a selected group of components and/or processes. More loosely optimization is used to mean improvement, even if a greater improvement might be obtainable. Many factors and outcomes, including but not limited to: effect on other processes, availability of an instrument, component or professional, cost, location, patient's wishes and government regulation may be factors in the optimization procedure and ultimate decisions made to determine a level of optimization. Optimization for one patient often will differ from optimization for another patient, but each patient will have improvement. Optimization may often involve improving one or more outcomes in concert with a possible worsening of another component of process.

Optimizing—The process of optimization. Optimization or the process is considered as a goal or a work in progress approaching an optimal or best outcome. Thus optimization may vary with time.

Organic—a compound containing carbon. A molecule having carbon and at least one other element.

Plectin—A protein found in several isoforms that is ubiquitous in the cytoskeleton of most mammalian cells. Plectin links actin microfilaments, microtubules and intermediate filaments (IF) together. Plectin also appears outside the cell in the extracellular linkages between cells.

Restore—to bring something to or towards a previous condition, a normal condition or an improved condition. The condition may be defined as a number or concentration, a rate of activity, a structure, or any observable or measurable process or product of metabolism.

Vimentin—VimIF, an intermediate filament protein that is involved in distribution, motility and anchoring of mitochondria. Vimentin can work with dyneins and actin-dependent myosins within the cell to deliver and anchor mitochondria close to where metabolic requirements are high.

The Mitochondrion: Optimization Target 1

Mitochondria, one of the organelles found in most eukaryotic cells are often called the “powerhouse” or “battery” of the cell. A eukaryotic cell typically has multiple mitochondria, the number being higher in cells with higher metabolisms. The molecule adenosine triphosphate (ATP) functions as a predominant energy carrier in the cell. Eukaryotic cells derive the majority of their ATP from biochemical processes carried out by their mitochondria. Within the cell mitochondria also tend to be found in regions with higher activities. Each cell has mechanisms to control mitochondrial synthesis and degradation and by balancing these mechanisms can control the number of mitochondria and metabolic rate of the cell. Cells also control movement of mitochondria so that their substrates and products can be efficiently delivered. Assisting the cells and organism containing the cells to optimize these activities will be found valuable in optimizing therapeutic outcomes.

These biochemical processes carried out by mitochondria include, but are not limited to the following important cycles: i) the citric acid cycle (the tricarboxylic acid cycle, or Krebs's cycle), generating reduced nicotinamide adenine dinucleotide (NADH+H⁺) from oxidized nicotinamide adenine dinucleotide (NAD⁺), and ii) oxidative phosphorylation, during which NADH+H⁺ is oxidized back to NAD⁺. (The citric acid cycle also reduces flavin adenine dinucleotide, or FAD, to FADH2; FADH2 also participates in oxidative phosphorylation.)

The respiratory chain of a mitochondrion is located in the inner mitochondrial membrane and consists of five multimeric protein complexes: Complex I; (approximately 44 subunits), Complex II (approximately 4 subunits), Complex III (approximately 11 subunits), Complex IV (approximately 13 subunits) and Complex V (approximately 16 units). (The reported number of subunits is given as approximate because the counts are different in different reports due to improving scientific understanding.) The respiratory chain also requires two small electron carriers, ubiquinone (coenzyme Q_{IO}) and cytochrome c.

ATP synthesis involves two coordinated processes: 1) electrons are transported horizontally from complexes I and II to coenzyme Q to Complex III to cytochrome c to Complex IV, and ultimately to the final electron acceptor, molecular oxygen, thereby producing water. At the same time, protons are pumped “vertically” across the mitochondrial inner membrane (i.e., from the matrix to the inter membrane space) by complexes I, II, II and IV. ATP is generated by the influx of these protons back into the mitochondrial matrix through complex V (mitochondrial ATP synthase). The energy released as these electrons traverse the complexes is used to generate a proton gradient across the inner membrane of the mitochondrion, which results in stored potential energy in the form of an electrochemical potential across the inner membrane.

In this process, Complex I (NADH dehydrogenase, also called NADH: ubiquinone oxidoreductase) removes two electrons from NADH and transfers them to a lipid-soluble carrier, ubiquinone. The reduced product, ubiquinol, is free to diffuse within the membrane.

At the same time, Complex I moves four protons (H⁺) across the membrane, producing a proton gradient. Complex I is one of the main sites at which premature electron leakage to oxygen occurs, thus being one of main sites of production of one harmful free radical called superoxide.

Complex II (succinate dehydrogenase) funnels additional electrons into the quinone pool by removing electrons from succinate and transferring them (via FAD) to the quinone pool. Complex H consists of four protein subunits: SDHA, SDHB, SDHC, and SDHD. Other electron donors (e.g., fatty acids and glycerol 3-phosphate) also funnel electrons into the quinone pool (via FAD), again without producing a proton gradient.

Complex III (cytochrome b/c complex) removes two electrons from OH2 and transfers them to two molecules of cytochrome c, the water-soluble electron carrier located between the membranes. As part of this process, it moves two protons across the membrane, producing a proton gradient (in total 4 protons: 2 protons are translocated and 2 protons are released from ubiquinol). When electron transfer is hindered (e.g., by a high membrane potential, point mutations or by respiratory inhibitors such as antimycin A), Complex III can leak electrons to oxygen resulting in the formation of superoxide, a highly-toxic oxidative species, which appears in the pathology of many diseases and is seen in aging.

Complex IV (cytochrome c oxidase) removes four electrons from four molecules of cytochrome c and transfers them to molecular oxygen (O₂), producing two molecules of water (H₂O). At the same time, it moves four protons (H⁺) across the membrane, producing a proton gradient.

Complex V (mitochondrial ATP synthetase) which is not directly associated with Complexes I, II, III and IV uses the energy stored by the electrochemical proton gradient to convert ADP into ATP.

McCormack et al. (2012) characterized one facet of mitochondrial disease as follows:
Mitochondrial respiratory chain disease is an increasingly well-recognized, but notoriously heterogeneous, group of multisystemic energy deficiency disorders. Its extensive heterogeneity has presented a substantial obstacle for establishing a definitive genetic diagnosis and clear pathogenic understanding in individual patients with suspected mitochondrial disease. While known genetic causes of “classical” mitochondrial DNA (mtDNA)—based disease syndromes have been readily diagnosable, the overwhelming majority of patients with clinical and/or biochemical evidence of suspected mitochondrial disease have had no identifiable genetic etiology for their debilitating or lethal disease. McCormick et al. 2012

To date about 10³ genes encoding mitochondrial proteins have been identified in humans (MitoCarta human inventory, Broad Institute). It is expected that most mammalian genomes will be quite similar. Mitochondrial dysfunction can arise from a mutation in one of these genes (causing a primary mitochondrial disorder) or from an outside influence on mitochondria (causing a secondary mitochondrial disorder). Mutations in 228 protein-encoding nDNA genes and 13 mtDNA genes have been linked to a human disorder with no reason to doubt that most mammals would not have similar evolutionary issues. The involvement of the activity of these genes in disorders emphasizes that optimizing function of any of these where they are found deficient can improve medical therapy.

Mitochondrial DNA is more prone to mutation effects in that the mitochondrion has a high rate of replication and lacks a DNA repair pathway in the organelle. The high level of active oxygens and the resultant oxidative stress also probably contribute to a relatively rapid mtDNA mutation rate. Thus, control of mtDNA mutation and control of mutated mtDNA can be important targets for optimization.

The present invention may target any one or more of these genes, control of these genes, expression products of these in optimization.

Common pharmaceutical drugs such as amiodarone, biguanides, haloperidol, statins, valproic acid, zidovudine, anesthetics, antibiotics, chemotherapeutic agents, and even NSAIDS like aspirin (acetylsalicylic acid) have been observed to affect total mitochondrial function. Given the multiple actions of drugs and their specificities for on and off target action, many drugs may lead more frequently to adverse reactions and side effects in patients with mitochondrial disorders than in otherwise healthy persons.

A recent search of Wikipedia (https://en.wikipedia.org/wiki/Mitochondrial_disease) accessed Jul. 7, 2015 and again Jul. 26, 2016 found the teaching:
Mitochondrial diseases are sometimes (about 15% of the time) used by mutations in the mtDNA that affect mitochondrial function. Other causes of mitochondrial disease are mutations in genes of the nDNA, whose gene products are imported into the Mitochondria (Mitochondrial proteins) as well as acquired mitochondrial conditions. Mitochondrial diseases take on unique characteristics both because of the way the diseases are often inherited and because mitochondria are so critical to cell function. The subclass of these diseases that have neuromuscular disease symptoms are often called a mitochondrial myopathy.

Mitochondrial Membranes as Structure

As previously mentioned, mitochondria contain two membranes. The outer mitochondrial membrane encompasses the inner membrane, with a small intermembrane space in between. The outer membrane has many protein-based pores that can allow the passage of simple ions and molecules as large as a small protein. In contrast, the inner membrane has much more restricted permeability. It is more like the plasma membrane of a cell. The inner membrane anchors proteins involved in electron transport and ATP synthesis. This membrane surrounds the mitochondrial matrix (the innermost compartment within the mitochondrion), where the citric acid cycle produces the electrons that travel from one protein complex to the next along the inner membrane. At the end of the ETC, the final electron acceptor is oxygen which ultimately forms water (H₂O). At the same time, the electron transport chain produces ATP. ADP (adenosine diphosphate) is phosphorylated to ATP (adenosine (triphosphate). (This is why the process is called oxidative phosphorylation.)

During electron transport, the participating protein complexes release protons from the matrix to the intermembrane space. This creates a concentration gradient of protons that another protein complex, Complex V, ATP synthase, uses to power synthesis of the energy carrier molecule ATP.

Although the mitochondrion has its own mtDNA, a vast majority of mitochondrial proteins are synthesized from nuclear genes (the DNA within another cell organelle, the cell nucleus) and transported into the mitochondria. These include, but are not limited to the enzymes required for the citric acid cycle, the proteins involved in DNA replication and transcription, and ribosomal proteins. The protein complexes of the respiratory chain are a mixture of proteins encoded by mitochondrial genes and proteins encoded by nuclear genes. Proteins in both the outer and inner mitochondrial membranes help transport newly synthesized, unfolded proteins from the cytoplasm into the matrix, where folding ensues.

Genetic Factors of Mitochondrial Proteins

Both nuclear and mitochondrial genes have been associated with disease by correlation with genetic mutation.

All 13 of the proteins encoded by the mitochondrial genome: MTND1, MTND2, MTND3, MTND4, MTND4L, MTND5, MTND6, MTCYB, MTCO1, MTCO2, MTCO3, MTATP6 and MTATP8, have mutations associated with disease. These proteins are generally found at mitochondrial inner membranes.

Nuclear genes encoding mitochondrial proteins (most likely found associated with or bound for the mitochondrial outer membrane) whose mutation has been linked to mitochondrial disease include but are not limited to: ARMS2, BCL2, CPT1A, DNM1L, GCK, GK, KIF1B, MAOA, PINK1.

Nuclear genes encoding mitochondrial proteins (most likely found associated with or bound for the mitochondrial inter membrane space) whose mutation has been linked to mitochondrial disease include but are not limited to: AK2, DIABLO, GATM, GFER, HTRA2, PANK2 and PPOX.

Nuclear genes encoding mitochondrial proteins (most likely found associated with or bound for the mitochondrial inner membrane) whose mutation has been linked to mitochondrial disease include but are not limited to: ABCB7, ACADVL, ADCK3, AGK, ATP5E, C12orf62, COX412, COX6B1, CPT2, CRAT, CYCS, CYP11A1, CYP11B1, CYP11B2, CYP24A1, CYP27A1, CYP27B1, DHODH, DNA1C19, FASTKD2, GPD2, HADHA, HADHB, HCCS, L2HGDH, MMAA, MPV17, NDUFA1, NDUFA2, NDUFA9, NDUFA10, NDUFA11, NDUFA12, NDUFA13, NDUFB3, NDUFB9, NDUFV1, NDUFV2, NDUFS1, NDUFS2, NDUFS3, NDUFS4, NDUFS6, NDUFS7, NDUFS8, OPA1, OPA3, PDSS1, SDHA, SDHB, SDHC, SDHD, SLC25A3, SLC25A4, SLC25A12, SLC25A13, SLC25A15, SLC25A19, SLC25A20, SLC25A22, SLC25A38, SPG7, TIMM8, UCP1, UCP2, UCP3, UQCRB and UQCRQ.

Nuclear genes encoding mitochondrial proteins (most likely found in or bound for the mitochondrial matrix) whose mutation has been linked to mitochondrial disease include but are not limited to: AARS2, ACAD8, ACAD9, ACADM, ACADS, ACADSB, ACAT1, ALAS2, ALDH2, ALDH4A1, ALDH6A1,AMT, ATPAF2, AUH, BCAT2, BCKDHA, BCKDHB, BCS1L, C8orf38, C10orf2, C12orf65, C20orf7, COA5, COX10, COX15, CPS1, D2HGDH, DARS2, DBT, DECR1, DGUOK, DLD, DLAT, DMGDH, ETFA, ETFB, ETFDH, FOXRED1, FH, GCDH, GCSH, GFM1, GLUD1, HADH, HARS2, HIBCH, HMGCS2, HMGCL, HSD17B10, HSPD1, IDH2, IDH3B, ISCU, IVD, KARS, MCCC1, MCCC2, MCEE, ME2, MRPS16, MRPS22, MTFMT, MTPAP, MUT, NAGS, NDUFAF1, NDUFAF2, NDUFAF3, NDUFAF4, NUBPL, OAT, OGDH, OTC, OXCT1, PC, PCCA, PCCB, PCK2, PDHA1, PDHB, PDHX, PDP1, POLG, POLG2, PYCR1, RARS2, RMRP, SARDH, SARS2, SCO1, SCO2, SDHAF1, SDHAF2, SOD2, SUCLA2, SUCLG1, SURF1, TACO1, TK2, TMEM70, TRMU, TSFM, TTC19, TUFM, UNG, XPNPEP3 and, YARS2.

Nuclear genes encoding mitochondrial proteins (but proteins that are also found in other places in the cell) whose mutation has been linked to mitochondrial disease include but are not limited to: AIFM1, AKAP10, AMACR, APTX, BAX, BOLA3, CYB5R3, ETHE1, FXN, GDAP1, HK1, HLCS, LRPPRC, LRRK2, MFN2, MLYCD, NFU1, PARK2, PARK7, SACS, SPG20 and WWOX.

Nuclear genes encoding mitochondrial proteins (but whose specific localization within the mitochondrion is still to be elucidated) whose mutation has been linked to mitochondrial disease include but are not limited to: GLRX5, HOGA1, MMAB, MMADHC, PDSS2, AFG3L2, COQ2, COQ6, COQ9, GLDC, PNKD, PUS1, REEP1, STAR and TMEM126A.

Functions of these genes and their products are known in the art. A listing of these genes and other information can be found, for example, in NEJM 2012; 366:1132-41, Supplementary Appendix, and is not repeated here.

Mitochondrial genes in general undergo post-translational modification. Accordingly, in the optimization process, embodiments of the present invention may modify any aspect of these genes, including but not limited to: any function associated with the gene, integrity of the gene itself, transcription, and all the factors including expression and post-translational modification and movement within the cell.

Since genetics underlie life functions, genes that do not encode a protein found in mitochondria can also be of extreme importance in optimized cell metabolism. For example, as discussed below the Position of mitochondria within cells is dependent on many proteins. The genes encoding these proteins can also be important in optimization.

Mitochondria are the major source of metabolic energy, and they regulate intracellular calcium levels and sequester apoptotic factors.

Mgm1 and Opa 1 are involved in regulating cristae structure. Mgm1 participates in ATPsynthase oligimerization.

Mitochondria are not just cell powerhouses producing ATP. They also are essential for other facets of cell functions required for metabolism. Cell metabolism is accomplished by thousands of enzymes. Many of these enzymes require metals for proper activity and to form coordination complexes. Iron sulfur clusters (ISVC), essential for iron homeostasis in the cell, are a product of mitochondria. Accordingly, mitochondria through this contribution to iron control, are necessary for many oxidation reactions, including, but not limited to: oxidative phosphorylation, pyrimidine/purine metabolism, the tricarboxylic acid cycle, acontinase activity, DNA repair, NTHL1 activity, heme synthesis, ferrochelatase function, ISC synthesis enzymes (NBP35 and CFD1). Metal containing enzymes, of which iron containing oxidation/reduction enzymes are common, are important for scavenging active oxygens. For example: FtMt is an important nuclear encoded mitochondrial protein that sequesters iron in mitochondria and makes it available when needed. Mdm33 is important for inner membrane fission. Proton pumping is coupled to ATP synthesis through F₁f₀ATP synthase.

Biochemicals

As observable from both FIGS. 1 and 2 and from reading this text, many biochemicals are essential or beneficial for proper cell metabolism. Depending on the contribution of the biochemical to the cell processes, the biochemical, may serve, for example, as a chemical substrate, a carrier, a structural member, a signal modifying activity of other biochemicals, etc. Changing location or activity of one may affect utilization of several others. Although not all of these intertwining pathways are mentioned in detail herein, any one or combination of the metabolic biochemicals and/or the biochemical enzymes processing them can be proper targets for optimization.

Targets, including, but not limited to:

Riboflavin (B₂)

L-Creatine

CoQ₁₀

L-arginine

L-carnitine

Vitamin C

Cyclosporin A

Manganese

Magnesium

Carnosine

Vitamin E

Resveratrol

Alpha lipoic acid

Folinic acid

Dichloroacetate

Succinate

Prostaglandins (PG)—specific to the PG and tissue may show positive/negative effect; e.g., PGA, PGA₂, PGB, PGB₂, PGC, PGD, PGD₂, PGE, PGE₁, PGE₂, PGE₃, PGF_{α}, PGF₁α, PGF₂α, PGF₃α, PGG, PGH, PGH₂, PGI, PGJ, PGK, and related biomolecules, including, but not limited to: prostacyclins, thromboxanes, prostanoic acid, 2-Arachidonoylglycerol, etc.

NSAIDS—aspirin—COX1 and COX2 inhibitors

Melatonin

Cocaine

Amphetamine

AZT and similar antiviral compounds

Mitophagic or mitophagic inhibitory compounds: including, but not limited to: isoborneol, piperine, tetramethylpyrazine, and astaxanthin

Glutathione

β-carotene and other carotenoids

and as further described below, to provide examples of optimization processing, are deliverable to cells and can be used in optimization as discussed in this application.

Some Representative Compounds and Their Importance

Riboflavin

Riboflavin (vitamin B2) works with the other B vitamins. It is important for body growth and red blood cell production and helps in releasing energy from carbohydrates.

L-creatine

Creatine is a naturally-occurring amino acid (protein building block) found in meat and fish, and also made in the liver, kidneys, and pancreas. It is converted into creatine phosphate or phosphocreatine and stored in the muscles, where it is used for energy. During high-intensity, short-duration exercise, such as lifting weights or sprinting, phosphocreatine is converted into ATP.

CoQ₁₀

There are two major factors that lead to deficiency of CoQ₁₀: reduced biosynthesis, and increased utilization by the body. Biosynthesis is the major source of CoQ₁₀. Biosynthesis requires at least 12 genes, and mutations in many of them are known to cause CoQ deficiency. CoQ₁₀ levels can also be affected by other genetic defects (such as mutations of mitochondrial DNA, ETFDH, APTX, FXN, and BRAF, genes that are not directly related to the CoQ₁₀ biosynthetic process)

Toxicity is not usually observed with high doses of CoQ₁₀. A daily dosage up to 3600 mg was found to be tolerated by healthy as well as unhealthy persons. However, some adverse effects, largely gastrointestinal, are reported with very high intakes.

L-Arginine

Arginine can be made by most mammals. However, normal biosynthetic pathways, produce insufficient amounts of arginine so some must still be consumed through diet. Arginine is the immediate precursor of nitric oxide (NO), urea, ornithine, and agmatine. Arginine is also a necessary precursor for the synthesis of creatine and other cell component biochemicals. The enzyme, arginase, is found in mitochondrial membranes and here contributes to proper function of the urea cycle. The metal, manganese, is also important for mitochondrial activity at least through its participation in arginine metabolism.

L-Carnitine

Carnitine is involved in the transport of acyl-coenzyme A across the mitochondrial membrane to be used in mitochondrial β-oxidation.

Vitamin C

Vitamin C reduces the exercise-induced expression of key transcription factors involved in mitochondrial biogenesis. These factors include peroxisome proliferator-activated receptor co-activator 1, nuclear respiratory factor 1, and mitochondrial transcription factor A. Vitamin C also prevented the exercise-induced expression of cytochrome C (a marker of mitochondrial content) and of the antioxidant enzymes superoxide dismutase and glutathione peroxidase. Vitamin C is an antioxidant, that along with resveratrol and alpha-lipoic acid reduces excessive reactive oxygen species production by the mitochondria. Manganese is also important here as vitamin C works with manganese superoxide dismutase.

Cyclosporin A

Cyclosporin A, an immune suppressant, interferes with the mitochondrial permeability transition pore and therefore has been found effective in protecting against oxidative stress in for example, stress inducing ischemic and reperfusion. Cyclosporin A can improve metabolism in some instances by slowing or blocking cell apoptosis.

Manganese

Manganese plays an essential role in the mitochondrial antioxidant: manganese superoxide dismutase. Without adequate manganese, superoxide dismutase activity will be insufficient, and therefore can result in sub-optimal mitochondrial activity and cellular metabolism.

Magnesium

Magnesium is important for proper calcium metabolism and function as a cofactor with many enzymes. Magnesium also appears especially important for mitochondrial biogenesis.

Zinc

Zinc is important in mitochondrial activity, for example, zinc can affect ATP production rates.

Carnosine

Carnosine is a potent scavenger of free radicals

Vitamin E

Vitamin F is a protectant against mitochondrial membrane peroxidation and therefore can be an important factor in maintaining mitochondrial activity and cellular metabolism.

Resveratrol

Resveratrol is a potent antioxidant with apparent involvement in mitochondrial biogenesis. Resveratrol acts through AMPK and SIRT1 and is involved in PGC-1α.

Alpha-Lipoic Acid

Alpha-lipoic acid is associated with rejuvenation and replacement of damaged mitochondria. This renewal becomes more prevalent as mitochondria age.

Folinic Acid

Folinic acid is a factor in mitochondrial oxidative stress and has been associated with mitochondrial dysfunction in autism spectrum.

Dichloroacetate (DCA)

DCA stimulates oxidative phosphorylation by inhibiting pyruvate dehydrogenase kinase. DCA has been investigated as a possible therapy in some cancers. DCA may also exert effects in the endocannabinolic system as a weak inverse agonist of CB₂.

Succinate

Succinate is an intermediate in the tricarboxylic acid cycle (making ATP), and participates in inflammatory signaling. Succinate dehydrogenase participates in electron transport as part of mitochondrial “Complex II”.

Prostaglandins (PG)

Calcium ion controls binding of many PGs to mitochondria thereby modifying many aspects of mitochondrial function. NSAIDS have been associated with decoupling activity in mitochondria.

NSAIDS—aspirin—COX1 and COX2 Inhibitors

NSAIDS are active in controlling mitochondrial Complex I. NSAIDS may also alter mitochondrial membrane permeability by opening the mitochondrial permeability transition pore that allows small molecules up to 1.5 kDa easier passage across the mitochondrial membrane.

Melatonin

Melatonin demonstrates cell protectant activity though slowing apoptosis as it controls activity of aged or oxidatively stressed mitochondria involvement in leading the cell down the apoptotic pathway.

Cocaine

The anesthetic, cocaine, has been observed as modifying Complex I activity in mitochondria.

Amphetamine

The stimulant class of amphetamines, are inhibitors or normal mitochondrial metabolism and appear to increase oxidative stress.

AZT and Similar Antiviral Compounds

AZT is mitochondrially active by increasing active oxygen generation, interacting with respiratory chain enzymes and damaging mtDNA. Thus optimization of mitochondrial function is a special need when drugs of this type are used.

Mitophagic or mitophagic inhibitory compounds: including, but not limited to: isoborneol, piperine, tetramethylpyrazine, and astaxanthin.

Mitophagy is important for recycling of mitochondria and controlling position and number of mitochondria. Either slowing or accelerating mitophagy may be important for optimizing metabolism in a particular cell or individual.

Glutathione

Increased glutathione is known to protect mitochondria and the cell against damaging effects of the oxidative moieties produced in mitochondria such as: superoxide anion radical O₂⁻, hydrogen peroxide, H₂O₂, and the extremely reactive hydroxyl radical *HO. Increasing intracellular glutathione content is possible by several methods including, but not limited to: supplying precursors for glutathione synthesis, e.g., N-acetylcysteine; increasing CoA, for example, by supplying its precursor pantothenic acid; making curcumin a spice) available to the cell; and the analgesic drug flupirtine. Since glutathione is seen to increase throughout the cell, the antioxidant protection is not limited to the mitochondria.

β-Carotene

β-carotene, lycopene, lutein, astaxanthin and zeaxanthin are popular carotenoids.

These biochemicals demonstrate antioxidation properties. These tend to be lipophilic and thus often are found partitioned in membranes. So at high concentrations they may disorganize normal membrane structure. Cautious treatment with one or more carotenoids can protect membranes against oxidative stress by inhibiting mitochondrial active oxygen production. At least in some cells carotenoids increase mitochondrial function while limiting active oxygen generation. Cell survival can be improved. If the cell whose health is improved is, for example, a cancer cell, then sometimes reduced carotenoids may be advantageous. Optimization here and with other modifications will depend on the disease, the individual and the cell and cell function targeted.

Mitochondrial Structure and Positioning

As more has been learned about mitochondria it is apparent they are dynamic organelles. From the earliest citing of the mitochondrion over a half century ago we now understand that mitochondrial shape and size are highly variable. Shape and size is controlled by fusion and fission processes. We can also observe that mitochondria are actively transported in cells depending on energy needs within the cell. More mitochondria become situated in areas with higher energy needs, including, but not limited to: active growth cones, presynaptic sites and postsynaptic sites. Also, the internal structure of mitochondria can change in response to their physiological state.

Shape. Length, shape, size and number of mitochondria are controlled by fusion and fission. Fusion will generally result in fewer, larger and more spherical mitochondria. Whereas high fission cells generally have more mitochondria that are smaller and rod shaped.

Outer shape is not the sole shape criterion. Mitochondria also have internal structure (e.g., shape of cristae). The cristae are regions of the inner membrane more distant (internal) from the outer membrane. Cristae are formed by internal folding of the inner membrane. The different portions of the inner membrane have different functions. For example, cristae are richer in oxidative phosphorylation machinery are more prevalent in cristae while transport facilitators are more prevalent in the inner membrane regions apposite the outer membrane. Not surprisingly, the density and length of cristae are controlled according to the cell's needs and the needs of specific location within the cell.

Location. One factor controlling mitochondrial movement is its membrane potential. Higher potential favors movement away from the cell nucleus or main cell body towards the periphery. Lower potential (possibly damaged mitochondria) migrate towards the cell center (possibly for destruction).

Signals such as a nerve growth factor (NGF) gradient act to recruit mitochondria to higher concentrations of NGF. These types of factors may be used as a piece of an optimization process to recruit mitochondria to targeted sites. Blocking nerve growth factor activity has been associated with bone cell necrosis.

Within the cell, mitochondria use the cytoskeleton as a guide to destination and for transportation.

Mitochondria are now known to migrate throughout cells, to fuse, and to divide as mitochondrial activity is regulated according to the cell's needs. The dynamic mitochondrial processes enable mitochondrial recruitment on demand to the changing more active subcellular compartments. Fusion processes as cells converge upon one another and merge facilitates content exchange between mitochondria and is a component of mitochondrial shape control. Stern cells which can fuse with endogenous cells may be involved in rescuing cells with damaged or otherwise dysfunctional mitochondria.

Movement is also important for mitochondrial communication with the cytosol and mitochondrial quality control. With these activities mitochondria readily adapt to changes in cellular requirements and therefore can respond to physiological or environmental imperatives. When mitochondrial dynamics becomes disrupted, cellular dysfunction ensues. Accordingly, optimization of cellular metabolism may involve modifying mitochondrial dynamics.

Optimization may thus involve consideration of the number of mitochondria, location of mitochondria, size of mitochondria, size and shape, internal structure of mitochondria in addition to chemical factors that may more specifically modify one or more mitochondrial function. Optimization of mitochondrial activity can be a valuable addition over and above the benefits obtained from balancing circulating androgen. Supplementation with cannabinoid active substances can facilitate the cells' and the organisms mitochondrial rebalancing.

Supplementing Levels of Androgen to Achieve Balance

As a side to the present invention, oral compositions, absorbable or metabolizable as the desired hormone can be incorporated into an animal's regular diet. Prohormones include dehydroepiandrosterone (DHEA), pregnenolone, androstenedione and/or androstenediol.

The oral composition may comprise the hormone itself or a suitable prohormone and may be delivered by any suitable means, including, but not limited to: as a toothpaste, a small treat, a food formulation, a prescription food product, a chew toy, etc.

The invention also provides methods of making the oral composition for enhancing human animal or animal-animal interaction, or simply animal health comfort and well-being by incorporating the hormone into one or more delivery devices.

One such device might be a toy, for example a dog toy shaped as a bone, a ring, a small animal, etc. Such device can be made in bulk using any conventional procedures, but for most accurate control of dosing for a specific animal may be produced in whole or in part using techniques commonly referenced as 3-D printing.

The 3-D printing procedure would incorporate a matrix suitable to maintain the animal's interest and print within this matrix, at least a portion of the toy with the hormone at the desired and exquisitely controlled concentration.

The matrix may start as a form produced using more bulk production processes and may be enhanced with one or more therapeutic areas such as a coating, a filler, a layer, etc., that comprises the hormone at the precisely determined dosing.

Non-oral delivery methods are available: for example, implantable devices similar in concept to identity chips or to birth control sticks or rods used by human females.

Rationale and Process

The present invention is based in part on the insight that administering one or more androgenic hormones or prohormones to generally healthy appearing animals can increase their overall health and beneficial interactions with nearby humans. In addition, though not as readily observable, achieving an optimal level of circulating hormone may be associated with an increased quality of life, possibly through enhancement of the immune system's ability to defend against bacteria and viruses, to resist cancer, to enhance the circulatory system, and/or to ameliorate undesired stress-responses. Co-administration of compounds active in the cannabinolic systems can fine-tune and facilitate such administrations.

Testosterone, since it is well studied and readily available is a preferred hormone to be used for controlling circulating androgenic hormone level and hormonal activity, though in some cases prohormones may have advantages. Known prohormones that are commercially available include: DHEA, pregnenolone, androstenedione, and androstenediol.

The compositions used in the present invention are preferably formulated and controllably administered to an animal to induce desired effects without also inducing undesired side effects, such as undesired anabolic or androgenic effects, in that animal.

Dose will depend on the initial status of circulating hormone in the animal, on the active ingredient and its activity within the animal, on the size of the animal, the frequency of dosing and the rate at which an animal would metabolize the active ingredient(s) of the composition. Thus suitable unit doses may range from about: 0.01 mg to 500 mg, e.g., 0.01 mg, 0.05 mg, 0.07 mg, 0.1 mg, 0.15 mg, 0.2 mg. 0.5 mg, 1.0 mg, 2 mg, 3 mg, 5 mg, 7 mg, 10 mg, 20 mg, 25 mg, 30 mg, 50 mg, 75 mg, 100 mg, 120 mg, 150 mg, 175 mg, 200 mg, 250 mg, 300 mg, 350 mg, 400 mg, 450 mg, or 500 mg. Depending on the route of delivery, the animal's size, the animal's age, the animal's gender, the specific composition, etc. other ranges may be relevant, for example, 10 mg-200 mg, 12.5 mg to 150 mg, 15 mg-100 mg, 20 mg-75 mg, 20 mg-50 mg, or 50 mg 100 mg.

Salts, esters, metabolites, protein bound, glycosylated, or matrixed formats of delivery a can be used in the composition, provided they are converted in vitro or in vivo to an active form. The composition may comprise hormone or pro-hormone that is bound, covalently or non-covalently to a non-hormonal substance.

The composition may optionally include additional vitamins or minerals and scents or flavorings or flavor enhancers to render the composition more acceptable to the administering human and/or to the animal. For example, beef, elk, chicken, salmon and/or other scent or flavor appealing to dogs, cats or other pets may be incorporated, a protein binder and/or a vitamin such as one of the B vitamins, e.g., B6 might be added as it or they might be found to help stabilize hormone level.

The composition may be made available in one or more formats including, but not limited to: a capsule, a tablet, a caplet, a liquid beverage, a powder, a liquid or powder beverage additive, a gel, a ready-to-eat food, either moist or dry, a chunk, a bar, a toy of desired shape and size. These will depend on the dosage required and acceptability to the animal and administering human.

A composition of the present invention may further comprise natural and/or artificial flavoring components, dyes or other coloring additives, preservatives and other conventional food supplement additives known in the art to increase palatability, storage options, etc.

The time and dosage amount administered will vary from animal to animal and will be influenced by the age of the subject, and therefore may be adjusted as the animal ages. It is believed that generally, the younger the animal, the earlier results will be apparent with a smaller dosage amount needed to achieve optimal results. As the animal ages, the composition will have to be administered perhaps more frequently and in larger dosages for the animal to experience optimal results.

The form of the oral composition can be any suitable form that comprises the active ingredient and allows delivery to the select animal. For example, preferably an animal has been under a veterinarian's care and is general good health. however, the animal is aging and can benefit from receiving a therapeutic intervention that while not strictly necessary for life is beneficial to the animal and its human interactions though optimizing health, for example, by staving off or diminishing arthritis, other bone issues, such as dysplasia, lessening obesity problems and other issues seen in aging animals, such as diabetes, lethargy, pain, etc.

Although the androgen in the oral composition may vary, the method of delivery is also an important factor. For example, testosterone may be co-administered with an oil, may be admixed in a feed, may be delivered as a toy, etc. The format for delivery is subject to choice of the animal caretakers and is manageable in accordance with this invention. Animals including humans have shown large variations in efficiencies of moving testosterone and other androgens from the gastro-intestinal track to circulation. Given the multiple effects of testosterone supplementation, most desirable effects being observed with approximately a doubling or tripling of typical circulating testosterone in a middle aged or older mammal, with more extreme levels risking undesirable androgenic consequences, monitoring testosterone delivery and testosterone or testosterone analogue in each individual is highly desired. Controlled and balanced delivery of the androgen supplement should be practiced to provide the best outcomes.

Accordingly, a preferred process in practicing the invention will utilize multiple tests performed over time and especially whenever feed is changed, perhaps when time of administration is changed, perhaps whenever the animal changes residences, and periodically as the animal increases in age.

Understanding that testosterone effects are found in the many tissues and organs expressing Androgen receptor and that depending on activity in each organ or tissue androgen may be degraded at different rates balancing testosterone levels may involve several iterative changes. So after an initial testosterone level determination and a choice of supplementation format and dose the animal will be supplemented for a period of time to achieve a new balance. After several weeks to several months the testosterone levels will again be monitored and the dosage or frequency of delivery adjusted to compensate for interbreed differences, differences in co-administered foodstuffs, and significantly factors specific to the specific animal. The balancing should receive periodic adjustment as endogenous androgen will be expected to diminish as the animal ages and degradation rates of testosterone will also fluctuate with age and other factors.

Cannabinoids may be used serially or coincident with corticosteroid or mitochondrial augmentation or rebalancing. The skilled artisan will be cognizant of the cannabinoid involvement in corticosteroid synthesis and release. When administration of one class is varied, the other classes may benefit from dosage or timing adjustment.

### EXAMPLE 2

For example, a five year old canine (age dependent on the breed, the animal size, etc.) is evaluated at its annual visit. This visit includes a hormonal profile as well as questioning the dog owner about the animal's activities and general health. The veterinarian observes that is common at this age for this type of dog, testosterone levels are continuing to drop and that the dog might benefit from restoring circulating levels of testosterone or other androgenic hormone in the blood.

The veterinarian calculates a target testosterone level and suggests simple oral supplements that can help the dog achieve these levels and to thereby pep up the dog, the dog owner and the dog's family.

The owner chooses from a brochure provided by the veterinarian one of the compositions of the present invention. In fact, the owner here wishes for variety and chooses a relatively hard chew toy, a gel format wherein the composition is encapsulated in a soft, bone shaped, gum like format that the owner believes her kids will enjoy giving the dog. She also takes a small food packet as a sample. This packet has two pouches and a small distribution device where a small quantifiable (by counting or volume measurement) portion (preferably slightly color coded for the human) can be admixed in prescribed proportion with the larger pouch contents to achieve the desired caloric intake and hormonal supplement dosage.

### EXAMPLE 3

Another example is a sibling of this dog. But this sibling has additional complications relating to itchy skin. The sibling also receives a filled bone treat structure wherein the inserted treat comprises a flavored cannabinolic active paste. This canine also receives five drops of active cannabinolic compounds with each of the twice daily feedings. In two days the scratching appears to be reduced and after two weeks the skin appears normal. The owner reduces the cannabinolic delivery to five drops once per day, but keeps replenishing the filled bones since the pet seemed to enjoy time spent chewing them.

Additional Benefit—Improving Energy Metabolism

As an additional benefit mitochondrial optimization might even more robustly improve the animal's general outlook. Combing mitochondrial optimization to optimize cellular metabolism in conjunction with androgen balancing is another feature that may further improve outcomes over and above the solely androgen focused approach.

Optimization of cellular metabolism through optimizing any mitochondrial function is desired for improved medical treatment. Cellular metabolism can be observed by any known method or any method that may become known and is not restricted to the examples discussed herein. However, examples are provided as a means to demonstrate the ubiquity of applications of the present invention and feasibility practicing it.

On a simplistic level mitochondrial function may be improved by what we might deem “appropriate nutrition”. Therapists and individuals have historically been known to supplement the diet with vitamins, nutrient and/or cofactors. To date, a methodologic approach to optimizing metabolism specific to an individual or group has not been practiced.

In many patients, more complete optimization will involve sequencing their mtDNA. The entire mitochondrial genome can be sequenced or select genes or regions might be deemed of greatest importance. Any one or more of the mitochondrial genes are candidates for sequencing. Sequencing is known in the art and can be accomplished by any successful methodology. Regions of particular interest including, but not limited to: the D-loop or control region might be sequenced to guide optimization protocols. Simply determining total mtDNA in a cell, tissue or individual may also be a step in optimization.

The mtDNA sequence results may be combined with genetic sequence information from one or more organs or cell types in an individual. Genomic sequence is one level of information that may be used in isolation or in combination with mtDNA sequence information for additional guidance in the optimization process. Even more robust information may be obtained, not just from gene expression profiling. This is very useful when considering specific organs or cell types which by being differentiated cells only express a small subset of the full genome. Obtaining RNA transcription profiles or expression profiles can thus be instrumental in the optimization process. In some circumstance analyzing proteins as discussed below with specific reference to blood and other ex vivo biopsy sources, can provide some genomic profile information by monitoring the end product of genomic expression. Accordingly, genomic information in isolation or more preferably in combination with clinical observation and other assays is understood to be a useful source of information to use in developing an optimization protocol.

Analysis may involve inhibiting certain mitochondrial functions to assess their performance levels. Also on occasion optimizing metabolism may involve mitochondrial inhibition.

Several examples of inhibitors are discussed as examples.

Electron Transport Chain Inhibitors

ETC inhibitors per se act by binding and blocking a component the electron transport chain. ETC function can also be inhibited by impairing expression or proper localization of one of the component enzymes or carriers. Inhibiting or blocking the ETC prevents electrons from being passed from one carrier to the next and stops oxidation of oxygen and synthesis of ATP. Since binding is involved the inhibitors act specifically to affect a particular carrier or complex. Binding can be temporary (reversible) or permanent (irreversible). Reversible inhibition may be time or concentration dependent. Irreversible inhibition generally results in total stoppage of respiration via the blocked pathway. Competitive inhibition is one form of reversible inhibition. It allows some oxygen consumption (and ATP synthesis) since a “trickle” of electrons can still pass through the blocked site. Although it allows some oxygen consumption, competitive inhibition may prevent maintenance of a chemiosmotic gradient. In this example, the addition of ADP would have no effect on respiration. Some combinations of inhibitors might be used to seek alternative entry points to the ETC.

Rotenone

Rotenone is used as an insecticide. It is toxic to wildlife and to humans as well as to insects. It is a competitive inhibitor of electron transport suitable for testing ability to block respiration via the NADH versus succinate pathway.

Antimycin

Antimycin has been used with combinations of substrates including succinate, NADH or glutamate, and the dye TMPD (N,N,N′,N′-tetramethyl-p-phenylenediamine) along with ascorbic acid.

Cyanide

Cyanide is a reversible inhibitor of cytochrome oxidase.

Some mitochondria have cyanide resistant pathways. Cyanide causes uncoupling. So in the presence of TMPD a dramatic increase in oxygen consumption is observable.

Malonate

Malonate is a competitive inhibitor of succinate metabolism.

Uncoupling Agents

Uncoupling is where the rate of electron transport is no longer be regulated by the chemiosmotic gradient. The condition is differentiated from electron transport inhibition by the fact that in the latter case, bypassing the block can restore the gradient. In uncoupling, the ETC still functions but is ineffective because of dissipation of the chemiosmotic gradient.

### 2,4-dinitrophenol (DNP)

DNP is a proton ionophore. It binds protons on one side of a membrane, and then being fat-soluble drifts to the opposite side where it loses the protons. The probability of binding is greatest on the side of the membrane with greatest proton concentration, and least on the side with the lesser concentration. This makes it impossible to maintain a proton gradient.

DNP demonstrates other effects in addition to uncoupling. DNP gradually inhibits electron transport itself as it incorporates into mitochondrial membranes. In the 1930s DNP was promoted as an effective diet pill. Uncoupling of electron transport from ATP synthesis allows rapid oxidation of Krebs substrates and promotes mobilization of carbohydrates and fats to maintain normal levels of the Krebs substances. The energy is lost and measurable as heat.

### Carbonyl cyanide p-[rifluoromethoxyl]-phenyl-hydrozone (FCCP)

FCCP is an ionophore, completely dissipating the chemiosmotic gradient while leaving the electron transport system uninhibited.

Oligomycin

Oligomycin, blocks ATP synthase by blocking the proton channel. This inhibits oxidative phosphorylation. Oligomycin has no effect on Complex IV respiration, but blocks Complex III respiration completely. It therefore has no direct effect on electron transport or the chemiosmotic gradient.

Any Mitochondrial Function or Related Function is a Possible Target for Optimization

Cells and mitochondria each and collectively require multiple metabolic functions for their own survival and survival of the organism. In any particular cell or condition, modifying a specific function or activity or a select group of metabolic functions or mitochondrial activities may be selected for optimization, in other cells or conditions, including, for example, cells of a different organ with the same individual or cells of a different individual. Such activities that might be altered associated with the optimization process may include but are not limited to: oxidative phosphorylation, energy versus heat production (efficiency), free radical generation, free radical scavenging, initiation of apoptosis, mtDNA transcription, mitochondrial protein translation, post translational modification, mitochondrial protein import or translocation, nucleotide translocation, ATP translocation, mitochondrial fission, mitochondrial fusion, Ca⁺⁺ compartmentalization or homeostasis, steroid biosynthesis, controlling portions of the urea cycle, fatty acid oxidation, the tricarboxylic acid cycle, pyruvate metabolism, cellular redox balance, synthesis of precursor compounds such as myelin precursors, altering iron metabolism and of course altering oxygen use and any component or activity of the electron transport chain. [Generation of metabolites to regulate cellular epigenetics (NAD⁺) methyl group and numerous additional metabolic processes.] The skilled artisan will recognize that optimization of any one or more of these may not be relevant for every cell type. Depending on the therapy at issue, any of these functions or activities or any of the many functions or activities not specifically mentioned here, but appropriate to the condition or cell involved in the treatment, the skilled artisan will select and optimize relevant functions and/or activities. To optimize treatment for the individual, disease status; the individual's history with the disease; the individual's response to the disease; the individual's genetic background (including methylation and other epigenetic control of polynucleic acids or their histones); the individual's biochemical status for one or more markers, metabolites or substrates; and experience such as data from the disease, the individual or any relevant group or subgroup can be used alone or in combination.

Cellular or mitochondrial morphology; e.g., size, number, location, shape, can be used to assess mitochondrial function. One means helpful in this analysis is FACS (fluorescence activated cell sorting). This technology is several decades old and therefore has seen development of a variety of fluorescent markers to indicate location, size, membrane potentials, including mitochondrial membrane potentials inside a cell. FACS is one technique available to assess deficits in mitochondrial form and/or function. Observing a facet of mitochondrial function that may be improved can be used to then select one or more optimizing strategies. Optionally, selected strategies can be tested in cells using repeated FACS, to refine and to further improve and optimize strategy.

Analysis of an individual or a group or class of individuals for normalization or validation can be directed explicitly at reactions carried out by mitochondria. However, this often may require a bioassay, removal of tissue from an individual for ex vivo analysis. And since the mitochondrion is an essential component of eukaryotic cells, participating in multiple metabolic pathways, mitochondrial status can be evaluated by secondary or tertiary parameters. For example, blood can be used to monitor mitochondrial health and therefore may be used in the present invention as a material for bioassay. Several fractions of blood may be used at the discretion of the practitioner. For example, mitochondria themselves can be found in white blood cells. Fibroblasts, mesenchymal stern cells, cancerous and/or cancer progenitor are examples of some rare but observable cell types that can be found in blood. Any cell found in the blood might be used as a source for nucleic acid to assay or sequence a nuclear or mitochondrial genome or a portion thereof.

The blood also carries other components, fatty acids, proteins, glycoproteins, lipoproteins, carbohydrates (simple and complex), gases (especially oxygen and carbon dioxide), ketones, hormones, metabolites, nitrogen compounds, active oxygen molecules, ions (atomic, polyatomic, organic, etc.), amino acids, plasma proteins (such as albumen that may scavenge [bind] drugs or other molecules), cytokines, platelets, molecules carried from the digestive system or lungs, etc. that may be used to indicate, tissue, cell and mitochondrial status. The invention envisages blood as a robust source of information that might be used in the optimization process. Each component may be assayed in its native or altered form. For example, a modified protein or nucleic acid can be very instructive in determining metabolic status. In many embodiments monitoring representative compounds as those discussed above will be useful in developing and monitoring optimization. In several embodiments cytokines, a generic term for interleukins including, but not limited to: IL-1a, IL-1b, IL1Rn, IL2, IL-3, IL-4, IL-5, IL-6, IL-7, IL-8, IL-9, IL-10, IL-11, II-12, IL12a, IL12b, IL-13, IL-14, IL-16, IL-17, IL-17a, IL17b, IL-18, IL-19, IL-20, II-21, IL-22, II-23, IL23a, IL-24, II-25, IL-26, II-27, II-28, IL-29, II-30, II-31, II-32, II-33, II-34, II-35, II-36, II-37, etc.), interferons(including, but not limited to: IFN-a, IFN-b, IFN-g, etc.), colony stimulating factors (including, but not limited to: M-CSF GM-CSF, G-CSF, [aka CSF1, CSF2 and CSF3] etc.), tumor necrosis factors (TNFA, Lymphotoxin (TNFB/LTA-TNFC/LTB), TNFSF4, TNFSF5/CD40LG, TNFSF6, TNFSF7, TNFSF8, TNFSF9, TNFSF10, TNFSF11, TNFSF13, TNFSF13B, EDA, etc.), and growth factors (including, but not limited to: BMP2, BMP4, BMP6, BMP7, CNTF, CNTF, GPI, LIF, MSTN, NODAL, OSM, THPO, VEGFA, etc. may be assessed to aid optimization.

Many drugs targeting cytokines or cytokine receptors have been developed or are under development. Accordingly, cytokine assays may be especially useful in developing optimization protocols since tools are available to modulate effect. Modulation of the endogenous quantities produced by an individual may be an enhancement tool used in some embodiments. Synthetic compounds antagonizing or agonizing of any assayed substance may also be appropriate tools.

Assaying any one blood component might crudely be used to monitor cellular and/or mitochondrial performance. However, there is no practical reason to eschew analysis of other components provide more directed information to guide optimization. Assaying multiple aspects can indicate performance or changed performance to judge an optimization pathway. For example, threshold levels of one or more blood components may indicate a certain level of activity of one or more metabolic pathway. Beyond simple thresholds, ratios of two or more components, by showing relationships, can provide more definitive information. Diurnal or other periodic relations may also guide optimization. Sometimes more complex algorithms getting at multi factor relationships (multiple pathways, serial pathways or parallel pathways, different organs, for example). Computer learning or other forms of artificial intelligence is now becoming a more accepted process to determine most effective analysis criteria.

While blood is a great source for a substantial number of components or factors that can be assayed, the body has other assayable tissues including, but not limited to: cerebral spinal fluid, lymph fluid, saliva, breath, tears, urine, sweat, mucus, gastric and/or intestinal contents, stool, etc. Any one or more of these tissues or components can be used individually or in conjunction with one or more other source to provide data used in optimization.

Analysis may be accomplished using any acceptable means such as categorization, parametric statistics, nonparametric statistics, ratio analysis, simple or complex comparisons, threshold assays, computer learning, etc.

Analysis may be repeated to assess degree of optimization and/or to assist in determining any change or addition to the optimization process. Analysis may also be repeated with any changed condition of the treatment recipient. Several repetitions of analysis and modified optimization process may be conducted in an iterative fashion.

Cellular metabolism or mitochondrial function may be optimized for an individual, even for an individual during a particular season, time of day, sleep-wake cycle, etc. Optimization may be based on data collected from more than one individual. For example, an optimized process may be determined for a select grouping. The skilled artisan will have capability to select an appropriate group, based for example on similarities within a group. If data show insignificant variability pooling is more appropriate.

Groupings may be based on disease or stage of disease. Groupings may be based on familial connections or larger genetic associations. For example, groups may be categorized from associations including, but not limited to: shared ancestry; shared country or region of familial origin; shared blood type (possibly subtypes); A, A1, A2, B, B1, etc.), shared Rh factor (possibly considering each or a combination of Cc, Dd, and Ee), any of the other grouping systems including, but not limited to: ABO, MNS, P, RH, LU, KEL, LE, FY, JK, DI, YT, XG, SC, DO, CO, L, CH, H, XK, GE, CROM, KN, IN, OK, RAPH, JMH, I, GLOB, GIL, RHAg, FORS, LAN, JR, Vel, CD59; HLA; one or more of the 4 main mitochondrial clusters with multiple DNA lineages; one or more of the 7 core mtDNA lineages (U, X, H, V, T, K, J); one or more of the nineteen mtDNA groups (A, B, C, D, F, G, H, I, J, K, L, M, N, U, V, W, and X); shared diet; shared eye color; shared gender; shared body type; similar height; similar weight; similar BMI or other biometric.

Generally, any assay might be used as part of the cell optimization process to assess one or more components of cell metabolism and/or mitochondrial activity. Some common types include but are not limited to: end point assays, kinetic assays, qualitative, semi-qualitative or quantitative assays, functional assays, immunoassays, radio-assays, fluorescent assays, binding assays, enzymatic assays, isotopic assays, mass spectrometry, photo-assays, MRI, PET, cell sort assays, spectrophotometry, polymerase chain reaction, laser coupled assays, agglutination assays, transmittance, absorbance, refraction, flow assays, size assays, ion assays, conductivity assays, uptake assays, secretion assays, mass, gel electrophoresis, transport of: DNA, RNA, proteins, or presence or amount of specific sequences, toxicity assays, viability assays, chemiluminescent assays, amino acid assays—amino acid ratio assays, carbohydrate analysis, biomarker assays, etc. Physiologic assessment may also be employed as an indicator of metabolism in progress. Production of reactive oxygen species, mitochondrial breakdown products or simply O₂ consumption may be used as indicators of mitochondrial performance and metabolism

Less specific assays can also be used to select optimization strategy. For example, fairly routine analysis of a biosubstance, e.g., a body fluid (for example: urine, blood, sweat, cerebral-spinal fluid, saliva) for one or more commonly seen components (for example: any of the amino acids, glucose or other monomeric compounds. One or more of the collagens may be observed to assess initial status and/or to monitor progression of the optimization strategy. For example, condition of the skin might be scored to chart effectiveness of treatment since skin is easily accessible and collagen is ubiquitous throughout the body's organs. As an example, collagen VI or a correlated marker might be monitored to assess Alzheimer's disease. Collagen monitoring may also be beneficial in tracking cancer growth and optimized treatment effectiveness. Assaying one or more biosubstance obtained, for example, from natural elimination or biopsy is considered important to many embodiments of the present invention.

This process of producing and properly distributing ATP for proper cell function is complex and therefore is sensitive to changes to the cell's homeostasis. Accordingly, a necessity for cell survival optimal function and energy metabolism (as manifest, for example, in FTC, protein or peptide synthesis, signal transduction, mitochondrial function, proton gradients and activated phosphates) is easily compromised before the therapy or during therapy that produces other desired effects. Accordingly, cell survival can be easily compromised; disruption to these processes can disruptively alter anything else, for example, post translational modification. Cellular energy metabolism needs to be optimized before or during therapy to maximize benefit.

As a description emphasizing complexity, the ETC incorporates three of these proton pumps known as complexes I, III and IV. Notably, complexes I and III catalyze reactions very close to equilibrium. Reactions catalyzed by these complexes are easily reversed and therefore especially sensitive to extracellular events.

Complex II can replace complex I, but is not a proton pump and produces less energy than pathways using complex I. When complex II becomes more active, energy metabolism and therefore the cell becomes less efficient.

Optimization therefore can have many possible pathways. One or more of these may be applied for any individual. For example, the mitochondrial genome encodes 37 genes (16, 569 bp): 13 polypeptides, 22 tRNAs and 2 ribosomal RNAs. The polypeptides are constituents of the respiratory-chair complexes: 7 complex I subunits (NADH dehydrogenase), 1 subunit of complex III (ubiquinol: cytochrome c oxidoreductase), 3 subunits of complex IV (cytochrome c oxidase) and 2 subunits of complex V (ATP synthase) The genes for tRNAs are presented as one-letter symbols. Mutations in four of these tRNA genes are associated with diabetes: those for leucine serine (S), lysine (K) and glutamic acid (E) tRNAs. [http://www.nature.com/nature/journal/v414/n6865/fig_tab/414807a_F1.html]. These, since the mitochondria are essential components of eukaryotic cells, interact with the cellular components produced by nuclear genome of the cell (since many pathways in energy production require genes from both).

Exemplary donor and acceptor compounds in the pathway include the coenzymes nicotinamide adenine dinucleotide (NAD⁺) and flavin adenine dinucleotide (FAD), yielding NADH and FADH₂. Then in the pathway, subsequent oxidation of these hydrogen acceptors leads to the production of ATP.

Since NADH is a component of the ETC, ETC and the mitochondrion are involved in other groups of pathways, for example reduction of disulfides. One such disulfide system is the glutathione system, a system essential for many transport functions within the cell and therefore healing and repair.

Even compounds such as fatty acids by participation in the citric acid cycle affect and/or are affected by any alteration from optimal mitochondrial function. So obesity or even localized fat deposition would be candidates for improvement through optimization of mitochondrial function.

To further highlight complexity of the energy system the following examples of molecules involved in energy metabolism are mentioned: carbohydrates, fats, proteins, acetyl-CoA, CoA-SH, cis-Aconitate, nicotinamide adenine dinucleotide (NAD⁺), reduced NAD⁺ (NADH), flavin adenine dinucleotide (FAD), FADH2, α-ketoglutarate, guanosine diphosphate (GDP), inorganic phosphate (Pi), guanosine triphosphate (GTP), adenosine diphosphate (ADP), adenosine triphosphate (ATP), hydronium and hydride ions, ubiquinone, and the reduced form ubiquinol, succinate, fumarate, Cytochrome c, isocitrate, oxalosuccinate, succinyl-CoA, L-malate and citrate. At a first level, every treatment altering any concentration, location, availability or enzymes that can use these substances as substrate would alter the energy metabolism set by the cell. In general, we should presume the metabolic balance set by the cell (in the absence of treatment) was optimized for at least one function. Restoring proper balance therefore should improve the treatment process.

On the downside of cell regulatory activities, in the past half century or so a class of molecules called reactive oxygen species (ROS) has been implicated in multiple disease etiologies. These are volatile oxygen substances that can initiate, for example, peroxidation chain reactions and may damage DNA as well as other cell components. Common diseases such as cardiovascular disease and many cancers are suspected as having ROS component in their development.

Mitochondrial function, because of its propensity to oxidize substances (chiefly involving oxygen) is therefore implicated in many disease states. Not surprisingly, many treatments for common disease will compromise mitochondrial function. Restoration of better health through optimizing energy metabolism should ideally become an important component of treatment.

In addition to merely optimizing mitochondrial function measured by optimizing the energy output, mitochondrial function may be optimized to treat or prevent some common disease. As mentioned above optimizing mitochondrial function to benefit proper glutathione levels can be considered important both for near term health and prevention or management of future disease.

Antioxidants, such as vitamins and red wines have been used generically, but generally not for specific effect to promote mitochondrial related health. Optimization of energy metabolism involves more than simply adding items to one's diet. Michael Ristow, in a 2009 study, found indeed that antioxidant supplementation (He used vitamins C and E.) had no positive effect. In fact, Ristow's studies were interpreted to conclude that antioxidant supplement left one weaker. So simply adding a molecule that counters an undesired molecule involved in mitochondrial metabolism is definitely not an obvious solution for ameliorating disease treatment or progression.

Enzymes, the catalysts for biologic activity, are important for optimized metabolism. Several of these enzymes require a metal to complete their structure. For example, superoxide dismutases (SODs) essential to detoxify active oxygens (like superoxide), contain either zinc Zn²⁺) and copper (Cu²⁺) or manganese (Mn²⁺) as in the mitochondrial form. These SODs convert superoxide to peroxide and thereby minimizes production of hydroxyl radical, the most potent of the oxygen free radicals. But the peroxides produced by SOD are also toxic. Peroxidase is the enzyme that detoxifies peroxides. The best-known mammalian peroxidase is glutathione peroxidase. This enzyme contains a modified amino acid selenocysteine in its reactive center.

This is perhaps understandable using, for example, Nrf2 as an exemplary intracellular regulator protein. Nrf2 activity is implicated in regulating a gross or more of gene in the cell. Optimization of mitochondrial function may affect Nrf2 activity on concomitantly, optimization of mitochondrial function may be addressed through controlling Nrf2.

Features

The invention involves many features that may be practiced individually or in combination. For example:
Feature 1. A composition for improving a non-human animal's well-being comprising an orally or subcutaneously administrable substance containing at least one dosage selected from the group consisting of: a mitochondrial booster, an androgen hormone and a prohormone, said dosage selected to optimize at least one physiologic function in a selected mammal.
Feature 2. The composition of feature 1 further comprising a compound having or supporting cannabinolic activity.
Feature 3. The composition of feature 2, comprising an endocannabinoid.
Feature 4. The composition of feature 2, wherein said activity is effected through a G-protein coupled receptor.
Feature 5. The composition of feature 2, wherein said activity is effected through a receptor selected from the group consisting of: CB₁, CB₂, TRPV₁, TRPV₂, TRPV₃, TRPV₄, TRPA₁, TRPM₈, GPR₁₈, GPR119, GPR₅₅ and GPR118.
Feature 6. The composition of feature 2, comprising a phytocannabinoid.
Feature 7. The composition of feature 2, comprising a synthetic cannabinoid.
Feature 8. The composition of feature 2 comprising a cannabinoid selected from the group consisting of: AEA, 2AG, PEA, OEA and LEA.
Feature 9. The composition of feature 2 comprising a cannabinoid selected from the group consisting of: URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A, KML29, MAFP, MJN110, ML30, N-arachidonoyl maleimide, OL-135, OL92, PF-04457845, SA-57, ST4070, URB880, URB937, indomethacin, MK-886, resveratrol, cis-resveratrol, aspirin, COX-1 inhibitor H, loganin, tenidap, SC560, FR 122047 hydrochloride, valeryl salicylate, FR122047 hydrate, ibuprofen, TFAP, 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)indomethacin amide,indomethacin heptyl ester, SC236, sulinac, sulindac sulfide, pravadoline, naproxen, naproxen sodium salt, meclofenamate sodium, ibuprofen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZLI-6, isoicam, dipyrone. YS121 and MEG (mercaptoethylguanidine).
Feature 10. The composition of feature 2 comprising a cannabinoid that is member of a class selected from the group consisting of: cannabigerol class, cannabichromene class, cannabicyclol class, Δ⁸-tetrahydrocannabinol class, cannabieson class, cannabinol and cannabinodiol class, cannabitriol class and miscellaneous class.
Feature 11. The composition of feature 2 comprising a cannabinoid selected from the group consisting of: CBGA, CBGAM, CBG, CBGM; CBGVA and CBGV.
Feature 12. The composition of feature 2 comprising a cannabinoid selected from the group consisting of: CBCA, CBC, CBCVA, CBCV, CBDA, CBD, CBDM, CBD-C4, CBDVA, CBDV, CBD-C1, THCA-A, THCA-B, 6a,10a-trans-6a,7,8,10a-tetrahydro-6,6,9-trimethyl-3-pentyl-6H-dibenzo[b,d]pyran-1-ol, THC,) THCA-C4, THC-C4, THCVA, THCV, Δ⁷-cis-isotetrahydro-cannabivarin, THCA-C1 and THC-C1.
Feature 13. The composition of feature 2 comprising a cannabinoid selected from the group consisting of: Δ⁸-TCA and Δ⁸-THC.
Feature 14. The composition of feature 2 comprising a cannabinoid selected from the group consisting of: CBL, CBLA and CBLV.
Feature 15. The composition of feature 2 comprising a cannabinoid selected from the group consisting of: CBEA-A, CBEA-B and CBE.
Feature 16. The composition of feature 2 comprising a cannabinoid selected from the group consisting of: CBNA, CBN, CBNM, CBN-C4, CBV, CBN-C2, CBN-C1), CBND and CBDV.
Feature 17. The composition of feature 2 comprising a cannabinoid selected from the group consisting of: CBT, 10-EHDT, 8,9-DHDT, CBTV and CBTVE.
Feature 18. The composition of feature 2 comprising a cannabinoid selected from the group consisting of: DCBF, CBF, CBCN, CBT, OTHC, cis-THC, 2H-iso-HHCV, CBR and triOH-THC.
Feature 19. The composition of feature 2 comprising a compound selected from the group consisting of: an FAAH inhibitor, R-WIN 55,212-2 and an MAGL inhibitor.
Feature 20. The composition of feature 2 comprising an EFA.
Feature 21. The composition of feature 2 comprising a fatty acid selected from the group consisting of: oxytocin, ω-3 fatty acid and ω-6 fatty acid.
Feature 22. The composition of feature 2 comprising a compound selected from the group consisting of: N-alkylamides, phytoalkanes, n-alkanes, N-acylethanolamines, flavonoids, curcuminoids, polyphenols, biphenyl neolignans, sesquiterpenes, N-Isobutylamides and p-hydroxyphenyl-O-arylcarbamates.
Feature 23. The composition of feature 22 comprising an alkane wherein said alkane has a number of carbons selected from the group consisting of: 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 and 39.
Feature 24. The composition of feature 22 comprising an alkane wherein said alkane comprises one or more sidegroups selected from the group consisting of: 2-methyl-, 3-methyl-, and dimethyl.
Feature 25. The composition of feature 2 comprising an alkane selected from the group consisting of: nonacosane, heptacosane, 2,6-dimethyltetradecane, pentacosane, hexacosane, and hentriacontane.
Feature 26. The composition of feature 2 comprising material derived from a plant selected from the group consisting of: Echinacea, Echinacea purpurea, Echinacea angustifolia, curcurmin, Salvia divinorum, sage, lemon grass, hops, verbana, Cannabis, thyme, mango, Helichrysum umbraculigerum liverwort, cacao, ginger, tumeric, Curcuma longa, Magnolia officinalis, Norway spruce, black pepper, basil, Myristica fragrans, cloves, Sciodopitys verticillata, oregano, cinnamon, black pepper, hemp, rosemary, flax and Elettaria repens.
Feature 27. The composition of feature 2 comprising a material selected from the group consisting of: β-caryophyllene, a β-caryophyllene oxide, salvinorin A, myrcene, perrottetinenic acid, apigenin, quercetin, cannflavin A, cannflavin B, β-sitosterol, vitexin, isovitexin, kaempferol, luteolin, orientin, a gingerol, capsaicin, curcumin, demethoxycurcumin, bisdemethoxycurcumin, cyclocurcumin, trans-resveratrol, diferuloylmethane, trans-arachidins, trans-piceatannol, isoprenylated trans-resveratrol derivatives, sciadonic acid magnolol, honokiol, malyngamide B, (+) sabinene, (−) sabinene, Isobutylamide, dodeca-2E,4E-dienoic acid isobutylamide, dodeca-2E,4E,8Z,10Z-tetraenoic acid alkylamide,1-[(2E,4E,8Z)-tetradecatrienoyl] piperidine, β-caryophyllene and ajulemic acid.
Feature 28. The composition of feature 2 wherein said compound comprises antioxidant activity.
Feature 29. The composition of feature 2 wherein said compound comprises a plant sourced composition selected from the group consisting of: abinene, α-pinene, 4,8-dimethyl-1,7-nonadien-4-ol, 2-hydroxy-4-methyl-valeric, acid, methyl, ester, octanal, O-cymene, eucalyptol, α-phellandrene, cis-sabinene, hydroxide, myrcenol, terpinen-4-ol, α-terpineol, β-thujene, ç-terpinene, trans-α-ocimene, carveol, β-citral, guanidine, geraniol, bornyl, acetate, β-pinene, thymol, geranic, acid, methyl, ester, α-terpinyl, acetate, d-limonene, eugenol, geranyl, acetate, dihydrocarvyl, acetate, α-ylangene, cis-dodec-5-enal, 3-phenyl-2-propenoic, acid, methyl, ester, β-elemene, c, vanillin, epoxy-α-terpenyl, acetate, butanoic, acid, 2-methyl-, 3,7-dimethyl-2,6-octadienyl, ester, 1-methyl-4-(1-acetoxy-1-methylethyl)-cyclohex-2-enol, 1,2,3,4,4a,5,6,8a-octahydro-4a,8-dimethyl-2-(1-methylethenyl)-, [2r-(2à,4aà,8aà)]-naphthalene, p-mentha-1(7),8-dien-2-ol, ç-muurolene, hydroxy-α-terpenyl, acetate, nerolidol, geranyl, bromide, (−)-α-panasinsen, pyrocatechol, ç-elemene, 9,10-dehydro-isolongifolene, à-calacorene, cis-verbenol, acetic, acid, 1-methyl-1-(4-methyl-5-oxo-cyclohex-3-enyl)ethyl, ester, alloaromadendrene, z,z-2,6-dimethyl-3,5,7-octatriene-2-ol, 4-epi-cubedol, 2-oxabicyclo[2.2.2]octan-6-ol, 1,3,3-trimethyl-acetate, patchoulane, farnesol, caryophyllene, oxide, cis-lanceol, ledene, oxide-(ii), farnesol, acetate, 6-epi-shyobunol, facarinol, phytol, aromadendrene, oxide-(2), heptacosane, longipinene, epoxide, hentriacontane, decamethyl-cyclopentasiloxane, geranyl, isobutyr, hexamethyl-cyclotrisiloxane, 1-docosene, tetratetracontane and dodecamethyl-cyclohexasiloxane.
Feature 30. A composition comprising a mitochondrial metabolic modifier in combination with a compound having or supporting cannabinolic activity.
Feature 31. The composition of feature 30 wherein said modifier is selected from the group consisting of: Riboflavin (B2), L-Creatine, CoQ₁₀, L-arginine, L-carnitine, vitamin C, cyclosporin A, manganese, magnesium, carnosine, vitamin F, resveratrol, α-lipoic acid, folinic acid, dichloroacetate, succinate, prostaglandins (PG) prostacyclins, thromboxanes, prostanoic acid, 2-arachidonoylglycerd and glutathione.
Feature 32. The composition of feature 30 wherein said compound having or supporting cannabinolic activity interacts with the pathway of a receptor selected from the group consisting of: CB₁, CB₂, TRPV₁, TRPV₂, TRPV₃, TRPV₄, TRPA₁, TRPM₈, GPR₁₈, GPR₁₁₉, GPR₅₅ and GPR₁₁₈.
Feature 33. A method for improving human emotional and/or metabolic well-being comprising increasing cannabinoid synthesis in said human through a targeted manipulation of a human-animal relationship.
Feature 34. The method of feature 33, wherein said targeted manipulation comprises administering to a pet or other human associate at least one compound selected from the group consisting of: a compound having or supporting cannabinolic activity, a compound having or supporting anabolic steroid activity and a compound comprising a mitochondrial metabolic modifier.
Feature 35. The composition of feature 1, comprising testosterone.
Feature 36. The composition of feature 1, wherein the physiologic function is selected from the group consisting of: sugar metabolism, joint health, bone density, blood pressure and caloric intake.
Feature 37. The composition according to feature 1 wherein the composition is formatted as a gel, a powder, a toy, a liquid, a food supplement, a moist food, a dry food, a small treat or a solidified matrix.
Feature 38. The method according to feature 1 wherein a 3-D printer is used to control dosing of the hormone or prohormone.
Feature 39. The composition according to feature 37 wherein the food comprises a plurality of packagings, wherein at least a first packaging contains active ingredient for admixing to at least a second package contents.
Feature 40. The composition according to feature 37 comprising a toy, wherein the toy is to a chewable substance.
Feature 41. The composition according to feature 40 wherein the chewable substance is shaped in a form selected from the group consisting of: a doggie bone, a dinosaur, a cat, a mouse, a squirrel, a rodent, a ring, a fist, a bow and a ball.
Feature 42. The composition according to feature 37, comprising a gel.
Feature 43. The composition according to feature 42 wherein the gel is incorporated into a dog toy.
Feature 44. The composition according to feature 1 wherein said at least one physiologic function is selected from the group consisting of: adipose metabolism, cardiac performance, glucose utilization, circulation, general nervous system activation or activity, hormonal secretion, salt (electrolyte) balance, function of a particular tissue or organ system, membrane transport across the membrane of one or more cell types, muscle activity, maintained muscle mass, O₂ consumption, skin health and visual abilities.
Feature 45. The composition according to feature 1 wherein the optimization of said at least one physiologic function has a result that improves at least one life factor selected from the group consisting of: general sense of well-being, clinical depression, fatigue sensation, athletic activity, positive interaction with another organism, motivation, liveliness and healing rate.
Feature 46. The composition according to feature 1 further comprising at least one promoter of mitochondrial metabolism.
Feature 47. The composition according to feature 46 wherein the facet of mitochondrial metabolism that is promoted is selected from the group consisting of: oxidative phosphorylation, coupling efficiency (energy versus heat production), free radical generation, free radical scavenging, initiation of apoptosis, mtDNA transcription, mtDNA maintenance, generation of reactive oxygen species, controlling DNA acetylation, controlling DNA methylation, histone modification, mitochondrial protein translation, post translational modification or mitochondrial proteins, mitochondrial protein import or translocation, ion import, ion homeostasis, nucleotide translocation, ATP translocation, mitochondrial fission, mitochondrial fusion, Ca⁺⁺ compartmentalization or homeostasis, steroid biosynthesis, a component of the urea cycle, fatty acid oxidation, a component of the tricarboxylic acid cycle, pyruvate metabolism, cellular redox balance, synthesis of precursor compounds for a mitochondrial function or activity, iron metabolism, oxygen metabolism and any component or activity of the electron transport chain.
Feature 48. The composition according to feature 1 further comprising a substance selected from the group consisting of: Riboflavin (B2), L-Creatine, CoQ₁₀, L-arginine, L-carnitine, vitamin C, cyclosporin A, manganese, magnesium, carnosine, vitamin E, resveratrol, α-lipoic acid, folinic acid, dichloroacetate, succinate, prostaglandins (PG) prostacyclins, thromboxanes, prostanoic acid, 2-arachidonoylglycerol and glutathione.
Feature 49. The composition according to feature 1 wherein the substance is complexed by covalent or non-covalent bonding with non-hormonal or non-prohormonal material.
Feature 50. The composition according to feature 1 wherein the substance further comprises at least one lipophilic vitamin.
Feature 51. The composition according to feature 50 wherein the at least one lipophilic vitamin is selected from the group consisting of: vitamin A, vitamin D, vitamin E and vitamin K.
Feature 52. The composition according to feature 1 wherein the substance is administered using a device implanted sub-dermally in the non-human animal.
Feature 53. The composition according to feature 52 wherein the composition is prepared using a 3D printing method.
Feature 54. A method for improving a non-human animal's well-being comprising: delivering to a selected non-human animal a dosage of androgen hormone or prohormone selected to improve at least one physiologic function in the selected animal.
Feature 55. The method according to feature 54 wherein:
   a) testosterone level is assessed in a non-human animal selected for possible benefit;
   b) an amount of testosterone is selected to increase circulating testosterone to a desired level and to improve at least one facet of the select animal's physiology;
   c) at least one composition is prepared, said composition comprising testosterone in an amount that taking into account the frequency and volume of said composition is designed to administer the amount selected in b); and
   d) administering said at least one composition in accordance with the volume and frequency of c).
Feature 56. The method according to feature 54 wherein said at least one physiologic function is selected from the group consisting of: adipose metabolism, cardiac performance, glucose utilization, circulation, general nervous system activation or activity, hormonal secretion, salt (electrolyte) balance, function of a particular tissue or organ system, membrane transport across the membrane of one or more cell types, muscle activity, maintained muscle mass, O₂ consumption, skin health and visual abilities.
Feature 57. The method according to feature 55 wherein after at least two weeks have elapsed from initiation of part d), parts a) through d) are repeated with an outcome that a desired androgen concentration remains is circulation.
Feature 58. The method according to feature 54 further comprising: delivering to said selected animal a promoter of mitochondrial function that is also selected to improve at least one physiologic function in the selected animal, said at least one physiologic function being identical to or differing from the at least one physiologic function targeted by the hormone or prohormone.
Feature 59. The method according to feature 58 wherein subsequent to mitochondrial function in said animal being assessed, said method further comprises choosing a substance to improve said mitochondrial function, and administering said substance to said animal.
Feature 60. The method according to feature 56 further comprising assessing mitochondrial function in said animal, choosing a substance to improve said mitochondrial function, and administering said substance to said animal.
Feature 61. The method according to feature 54 wherein the at least one composition comprises one selected from the group consisting of: beef, elk, chicken, salmon, protein binder and a vitamin.
Feature 62. The method according to feature 54 wherein the at least one composition comprises one in a form selected from the group consisting of: a capsule, a tablet, a caplet, a liquid beverage, a powder, a liquid or powder beverage additive, a gel, a ready-to-eat food, either moist or dry, a chunk, a bar and a toy,
Feature 63. A method for improving well-being in a non-human animal, said method comprising increasing circulating levels of SHBG protein in the blood.
Feature 64. The method according to feature 63 wherein SHBG is produced by enteric organisms cultured into the animal's microbiome.
Feature 65. The method according to feature 63 wherein a SHBG porous capsule is introduced subcutaneously, said capsule comprising in its interior an expression system producing SHBG.
Feature 66. The method according to feature 63 wherein the animal is genetically modified to increase SHBG expression.
Feature 67. The method according to feature 63 comprising a lipoporous capsule is introduced subcutaneously, said capsule comprising in its interior a system releasing androgenic substance capable of binding with SHBG.
Feature 68. The method of feature 54 further comprising delivering to said animal a compound having or supporting cannabinolic activity,
Feature 69. A method for improving a non-human animal's well-being comprising administering to said animal at least one mitochondrial metabolic booster selected from the group consisting of: Riboflavin (B2), L-Creatine, CoQ₁₀, L-arginine, L-carnitine, vitamin C, cyclosporin A, manganese, magnesium, carnosine, vitamin E, resveratrol, α-lipoic acid, folinic acid, dichloroacetate, succinate, prostaglandins (PG) prostacyclins, thromboxanes, prostanoic acid, and glutathione in association with administering to said animal at least one compound having or supporting cannabinolic activity.
Feature 70. A method for improving a non-human animal's well-being comprising: administering to said animal at least one composition designed to impact a system or output selected from the group consisting of: oxidative phosphorylation, coupling efficiency (energy versus heat production), free radical generation, free radical scavenging, initiation of apoptosis, mtDNA transcription, mtDNA maintenance, generation of reactive oxygen species, controlling DNA acetylation, controlling DNA methylation, histone modification, mitochondrial protein translation, post translational modification or mitochondrial proteins, mitochondrial protein import or translocation, ion import, ion homeostasis, nucleotide translocation, ATP translocation, mitochondrial fission, mitochondrial fusion, Ca⁺⁺ compartmentalization or homeostasis, steroid biosynthesis, a component of the urea cycle, fatty acid oxidation, a component of the tricarboxylic acid cycle, pyruvate metabolism, cellular redox balance, synthesis of precursor compounds for a mitochondrial function or activity, iron metabolism, oxygen metabolism and any component or activity of the electron transport chain in said animal; and balancing said impact through administering to said animal at least one compound having or supporting cannabinolic activity.
Feature 71. A method for improving a non-human animal's well-being comprising administering to said animal at least one compound having or supporting cannabinolic activity for rebalancing at least one physiologic system in said animal and wherein said animal has been recognized as one as likely to benefit from such rebalancing.
Feature 72. The method of feature 71 wherein said at least one compound comprises one or more compounds selected from the group consisting of: CBGA, CBGAM, CBG, CBGM; CBGVA and CBGV.
Feature 73. The composition of claim 2 comprising a cannabinoid selected from the group consisting of: CBCA, CBC, CBCVA, CBCV, CBCA, CBD, CBDM, CBD-C4, CBDVA, CBCV, CBD-C1, THCA-A, THCA-B, 6a,10a-trans-6a,7,8,10a-tetrahydro-6,6,9-trimethyl-3-pentyl-6H-dibenzo[b,d]pyran-1-ol, THC,) THCA-C4, THC-C4, THCVA, THCV, Δ⁷-cis-isotetrahydrocannabivarin, THCA-C1, THC-C1, Δ⁸-TCA, Δ⁸-THC, CBL, CBLA, CBLV, CBEA-A, CBEA-B, CBE, CBNA, CBN, CBNM, CBN-C4, CBV, CBN-C2, CBN-C1), CBND, CBDV, CBT, 10-EHDT, 8,9-DHDT, CBTV, CBTVE, DCBF, CBF, CBCN, CBT, OTHC, cis-THC, 2H-Iso-HHCV, CBR, triOH-THC, abinene, α-pinene, 4,8-dimethyl-1,7-nonadien-4-ol, 2-hydroxy-4-methyl-valeric, acid, methyl, ester, octanal, O-cymene, eucalyptol, α-phellandrene, cis-sabinene, hydroxide, myrcenol, terpinen-4-ol, α-terpineol, β-thujene, ç-terpinene, trans-α-ocimene, carveol, β-citral, guanidine, geraniol, bornyl, acetate, β-pinene, thymol, geranic, acid, methyl, ester, α-terpinyl, acetate, d-limonene, eugenol, geranyl, acetate, dihydrocarvyl, acetate, α-ylangene, cis-dodec-5-enal, 3-phenyl-2-propenoic, acid, methyl, ester, β-elemene, c, vanillin, epoxy-α-terpenyl, acetate, butanoic, acid, 2-methyl-, 3,7-dimethyl-2,6-octadienyl, ester, 1-methyl-4-(1-acetoxy-1-methylethyl)-cyclohex-2-enol, 1,2,3,4,4a,5,6,8a-octahydro-4a,8-dimethyl-2-(1-methylethenyl)-, [2r-(2à,4aà,8aà)]-naphthalene, p-mentha-1(7),8-dien-2-ol, ç-muurolene, hydroxy-α-terpenyl, acetate, nerolidol, geranyl, bromide, (−)-α-panasinsen, pyrocatechol, ç-elemene, 9,10-dehydro-isolongifolene, à-calacorene, cis-verbenol, acetic, acid, 1-methyl-1-(4-methyl-5-oxo-cyclohex-3-enyl)ethyl, ester, alloaromadendrene, z,z-2,6-dimethyl-3,5,7-octatriene-2-ol, 4-epi-cubedol, 2-oxabicyclo[2.2.2]octan-6-ol, 1,3,3-trimethyl-acetate, patchoulane, farnesol, caryophyllene, oxide, cis-lanceol, ledene, farnesol, acetate, 6-epi-shyobunol, falcarinol, phytol, aromadendrene, oxide-(2), heptacosane, longipinene, epoxide, hentriacontane, decamethyl-cyclopentasiloxane, geranyl, isobutyr, hexamethyl-cyclotrisiloxane, 1-docosene, tetratetracontane, dodecamethyl-cyclohexasiloxane, URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A, KML29, MAFP, MJN110, ML30, N-arachidonoyl maleimide, OL-135, OL92, PF-04457845, SA-57, ST4070, URB880, URB937, indomethacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen, TFAP, 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)indomethacin amide, indomethacin heptyl ester, SC236, sulinac, sulindac sulfide, pravadoline, naproxen, naproxen sodium salt, meclofenamate sodium, ibuprofen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZLI-6, isoicam, dipyrone, YS121 and MEG (mercaptoethylguanidine).

In concert with the above discussion, we need to remember that the mammalian organisms, including the mitochondria that reside in its cells, have evolved over eons. It is only recently that humans have used medicinal sciences to target invading organisms, dysfunctioning organs or cells, messaging pathways dictating cell activity, or cells' internal components and functions. While often animals will have evolved to improve or optimize natural stresses, these newly manufactured stresses will not be managed by systems that through trial and error (evolution) have been optimized to a degree to maximize survival of the species. When a disease affects an organism or with good intentions we presume to modify one part of the cell's activities, because of the interrelatedness of the multiple pathways within a cell, we likely will observe secondary and tertiary or more abstract effects if we look for them. Investigating whether such an important component of the cell, such as the mitochondrion, can have its function improved and taking action to improve function can be expected to show great benefits to the individual.

Mitochondrial function is thus extremely important and changeable. Any mitochondrial gene or any mitochondrial protein gene, their control mechanisms and their products or metabolites should therefore be considered as possible targets in the optimization processes. For example, Slowing MFN1, MFN2 or OPA1 can seriously reduce respiratory capacity. Combination of multiple modifying schemes sometimes can be quite advantageous. For example, generic components, such as lipids (including glycolipids, phospholipids, etc.), substrates, and possibly indicator substances might be introduced while also increasing mitochondrial fusion. The fusion aids in more widespread distribution and delivery. When movement is the goal, increasing fission can make the mitochondria more mobile and enable delivery to cell periphery. Fission is also a facilitator of apoptosis. Accordingly, increasing fission events can aid treatments where apoptosis is desired and decreasing fission can spare cell death.

Combined interventions involving two or more episodes of redirecting/rebalancing immune/allergy activity, balancing androgen levels in circulation improving animal activity and outlook by optimizing mitochondrial activities will allow even more robust human-animal interaction and better outcomes for the participants.

Accordingly, one embodiment of the invention might include additional assays. These additional assays would relate to optimizing mitochondrial and cellular performance taking into account the changed and improved organism and cellular activities the result from balancing the androgens.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In the case of conflict, the present specification, including definitions, will control. In addition, the materials, methods, and examples are illustrative only and not intended to be limiting. Although for simplicity in drafting the claims below are drafted in a manner where each dependent claim specifically asserts dependent status with only a single claim, the reader is put on notice that for purpose of disclosure every claim that references a preceding claim also implicitly is understood to have alternate dependency to all claims ultimately depending from the same claim or claims.

Of course the reader will recognize that this implicit understanding will not be stretched ridiculously to the extent that a claim might depend from itself. For example, claim 22 which explicitly references claim 2 and no other claim is interpreted as disclosing reference to claims 2-32 (and claim 1 through claim 2); claim 22 which is referenced in claims 23 and 24 is interpreted as disclosing reference to other codependency claims but not claims 23 and 24 which themselves reference claim 22. Other features and advantages of the invention will be apparent from the following detailed description and claims.

1. A composition for improving a non-human animal's well-being comprising an orally or subcutaneously administrable substance containing at least one dosage selected from the group consisting of: a mitochondrial booster, an androgen hormone and a prohormone, said dosage selected to optimize at least one physiologic function in a selected mammal.

2. The composition of claim 1 further comprising a compound having or supporting cannabinolic activity.

3. The composition of claim 2, comprising an endocannabinoid.

4. The composition of claim 2, wherein said activity is effected through a G-protein coupled receptor.

5. The composition of claim 2, wherein said activity is effected through a receptor selected from the group consisting of: C₁, CB₂, TRPV₁, TRPV₂, TRPV₃, TRPV₄, TRPA₁, TRPM₈, GPR₁₈, GPR₁₁₉, GPR₅₅ and GPR₁₁₈.

6. The composition of claim 2, comprising a phytocannabinoid.

7. The composition of claim 2, comprising a synthetic cannabinoid.

8. The composition of claim 2 comprising a cannabinoid selected from the group consisting of: AEA, 2AG, PEA, OEA and LEA.

9. The composition of claim 2 comprising a cannabinoid selected from the group consisting of: URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A, KML29, MAFP, MJN110, ML30, N-arachidonoyl maleimide, OL-135, OL92, PF-04457845, SA-57, ST4070, URB880, URB937, indomethacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen, TFAP, 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)indomethacin amide,indomethacin heptyl ester, SC236, sulinac, sulindac sulfide, pravadoline, naproxen, naproxen sodium salt, meclofenamate sodium, ibuprofen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZLI-6, isoicam, dipyrone, YS121 and MEG (mercaptoethylguanidine).

10. The composition of claim 2 comprising a cannabinoid that is member of a class selected from the group consisting of: cannabigerol class, cannabichromene class, cannabicyclol class, Δ⁸-tetrahydrocannabinol class, cannabieson class, cannabinol and cannabinodiol class, cannabitriol class and miscellaneous class.

11. The composition of claim 2 comprising a cannabinoid selected from the group consisting of: CBGA, CBGAM, CBG, CBGM; CBGVA and CBGV.

12. The composition of claim 2 comprising a cannabinoid selected from the group consisting of: CBCA, CBC, CBCVA, CBCV, CBDA, CBD, CBDM, CBD-C4, CBDVA, CBDV, CBD-C1, THCA-A, THCA-B, 6a,10a-trans-6a,7,8,10a-tetrahydro-6,6,9-trimethyl-3-pentyl-6H-dibenzo[b,d]pyran-1-ol, THC,) THCA-C4, THC-C4, THCVA, THCV, Δ⁷-cis-isotetrahydro-cannabivarin, THCA-C1 and THC-C1.

13. The composition of claim 2 comprising a cannabinoid selected from the group consisting of: Δ⁸-TCA and Δ⁸-THC.

14. The composition of claim 2 comprising a cannabinoid selected from the group consisting of: CBL, CBLA and CBLV.

15. The composition of claim 2 comprising a cannabinoid selected from the group consisting of: CBEA-A, CBEA-B and CBE.

16. The composition of claim 2 comprising a cannabinoid selected from the group consisting of: CBNA, CBN, CBNM, CBN-C4, CBV, CBN-C2, CBN-C1), CBND and CBDV.

17. The composition of claim 2 comprising a cannabinoid selected from the group consisting of: CBT, 10-EHDT, 8,9-DHDT, CBTV and CBTVE.

18. The composition of claim 2 comprising a cannabinoid selected from the group consisting of: DCBF, CBF, CBCN, CBT, OTHC, cis-THC, 2H-iso-HHCV, CBR and triOH-THC.

19. The composition of claim 2 comprising a compound selected from the group consisting of: an FAAH inhibitor, R-WIN 55,212-2 and an MAGL inhibitor.

20. The composition of claim 2 comprising an EFA.

21. The composition of claim 2 comprising a fatty acid selected from the group consisting of: oxytocin, ω-3 fatty acid and ω-6 fatty acid.

22. The composition of claim 2 comprising a compound selected from the group consisting of: N-alkylamides, phytoalkanes, n-alkanes, N-acylethanolamines, flavonoids, curcuminoids, polyphenols, biphenyl neolignans, sesquiterpenes, N-Isobutylamides and p-hydroxyphenyl-O-arylcarbamates.

23. The composition of claim 22 comprising an alkane wherein said alkane has a number of carbons selected from the group consisting of: 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 and 39.

24. The composition of claim 22 comprising an alkane wherein said alkane comprises one or more sidegroups selected from the group consisting of: 2-methyl-, 3-methyl-, and dimethyl.

25. The composition of claim 2 comprising an alkane selected from the group consisting of: nonacosane, heptacosane, 2,6-dimethyltetradecane, pentacosane, hexacosane, and hentriacontane.

26. The composition of claim 2 comprising material derived from a plant selected from the group consisting of: Echinacea, Echinacea purpurea, Echinacea angustifolia, curcurmin, Salvia divinorum, sage, lemon grass, hops, verbana, Cannabis, thyme, mango, Helichrysum umbraculigerum liverwort, cacao, ginger, tumeric, Curcuma longa, Magnolia officinalis, Norway spruce, black pepper, basil, Myristica fragrans, cloves, Sciadopitys verticillata, oregano, cinnamon, black pepper, hemp, rosemary, flax and Elettaria repens.

27. The composition of claim 2 comprising a material selected from the group consisting of: β-caryophyllene, a β-caryophyllene oxide, salvinorin A, myrcene, perrottetinenic acid, apigenin, quercetin, cannflavin A, cannflavin B, β-sitosterol, vitexin, isovitexin, kaempferol, luteolin, orientin, a gingerol, capsaicin, curcumin, demethoxycurcumin, bisdemethoxycurcumin, cyclocurcumin, trans-resveratrol, diferuloylmethane, trans-arachidins, trans-piceatannol, isoprenylated trans-resveratrol derivatives, sciadonic acid magnolol, honokiol, malyngamide B, (+) sabinene, (−) sabinene, Isobutylamide, dodeca-2E,4E-dienoic acid isobutylamide, dodeca-2E,4E,8Z,10Z-tetraenoic acid alkylamide, 1-[(2E,4E,8Z)-tetradecatrienoyl] piperidine, β-caryophyllene and ajulemic acid

28. The composition of claim 2 wherein said compound comprises antioxidant activity.

29. The composition of claim 2 wherein said compound comprises a plant sourced composition selected from the group consisting of: abinene, α-pinene, 4,8-dimethyl-1,7-nonadien-4-ol, 2-hydroxy-4-methyl-valeric, acid, methyl, ester, octanal, O-cymene, eucalyptol, α-phellandrene, cis-sabinene, hydroxide, myrcenol, terpinen-4-ol, α-terpineol, β-thujene, ç-terpinene, trans-α-ocimene, carveol, β-citral, guanidine, geraniol, bornyl, acetate, β-pinene, thymol, geranic, acid, methyl, ester, α-terpinyl, acetate, d-limonene, eugenol, geranyl, acetate, dihydrocarvyl, acetate, α-ylangene, cis-dodec-5-enal, 3-phenyl-2-propenoic, acid, methyl, ester, β-elemene, c, vanillin, epoxy-α-terpenyl, acetate, butanoic, acid, 2-methyl-, 3,7-dimethyl-2,6-octadienyl, ester, 1-methyl-4-(1-acetoxy-1-methylethyl)-cyclohex-2-enol, 1,2,3,4,4a,5,6,8a-octahydro-4a,8-dimethyl-2-(1-methylethenyl)-, [2r-(2à,4aà,8aà)]-naphthalene, p-mentha-1(7),8-dien-2-ol, ç-muurolene, hydroxy-α-terpenyl, acetate, nerolidol, geranyl, bromide, (−)-α-panasinsen, pyrocatechol, ç-elemene, 9,10-dehydro-isolongifolene, à-calacorene, cis-verbenol, acetic, acid, 1-methyl-1-(4-methyl-5-oxo-cyclohex-3-enyl)ethyl, ester, alloaromadendrene, z,z-2,6-dimethyl-3,5,7-octatriene-2-ol, 4-epi-cubedol, 2-oxabicyclo[2.2.2]octan-6-ol, 1,3,3-trimethyl-acetate, patchoulane, farnesol, caryophyllene, oxide, cis-lanceol, ledene, oxide-(ii), farnesol, acetate, 6-epi-shyobunol, falcarinol, phytol, aromadendrene, oxide-(2), heptacosane, longipinene, epoxide, hentriacontane, decamethyl-cyclopentasiloxane, geranyl, isobutyr, hexamethyl-cyclotrisiloxane, 1-docosene, tetratetracontane and dodecamethyl-cyclohexasiloxane.

30. A composition comprising a mitochondrial metabolic modifier in combination with a compound having or supporting cannabinolic activity.

31. The composition of claim 30 wherein said modifier is selected from the group consisting of: Riboflavin (B2), L-Creatine, CoQ₁₀, L-arginine, L-carnitine, vitamin C, cyclosporin A, manganese, magnesium, carnosine, vitamin E, resveratrol, α-lipoic acid, folinic acid, dichloroacetate, succinate, prostaglandins (PG) prostacyclins, thromboxanes, prostanoic acid, 2-arachidonoylglycerol and glutathione.

32. The composition of claim 30 wherein said compound having or supporting cannabinolic activity interacts with the pathway of a receptor selected from the group consisting of: CB₁, CB₂, TRPV₁, TRPV₂, TRPV₃, TRPV₄, TRPA₁, TRPM₈, GPR₁₈, GPR₁₁₉, GPR₅₅ and GPR₁₁₈.

33. A method for improving human emotional and/or metabolic well-being comprising increasing cannabinoid synthesis in said human through a targeted manipulation of a human-animal relationship.

34. The method of claim 33, wherein said targeted manipulation comprises administering to a pet or other human associate at least one compound selected from the group consisting of: a compound having or supporting cannabinolic activity, a compound having or supporting anabolic steroid activity and a compound comprising a mitochondrial metabolic modifier.

35. The composition of claim 1, comprising testosterone.

36. The composition of claim 1, wherein the physiologic function is selected from the group consisting of: sugar metabolism, joint health, bone density, blood pressure and caloric intake.

37. The composition according to claim 1 wherein the composition is formatted as a gel, a powder, a toy, a liquid, a food supplement, a moist food, a dry food, a small treat or a solidified matrix.

38. The method according to claim 1 wherein a 3-D printer is used to control dosing of the hormone or prohormone.

39. The composition according to claim 37 wherein the food comprises a plurality of packagings, wherein at least a first packaging contains active ingredient for admixing to at least a second package contents.

40. The composition according to claim 37 comprising a toy, wherein the toy is a chewable substance.

41. The composition according to claim 40 wherein the chewable substance is shaped in a form selected from the group consisting of: a doggie bone, a dinosaur, a cat, a mouse, a squirrel, a rodent, a ring, a fist, a bow and a ball.

42. The composition according to claim 37, comprising a gel.

43. The composition according to claim 42 wherein the gel is incorporated into a dog toy.

44. The composition according to claim 1 wherein said at least one physiologic function is selected from the group consisting of: adipose metabolism, cardiac performance, glucose utilization, circulation, general nervous system activation or activity, hormonal secretion, salt (electrolyte) balance, function of a particular tissue or organ system, membrane transport across the membrane of one or more cell types, muscle activity, maintained muscle mass, O₂ consumption, skin health and visual abilities.

45. The composition according to claim 1 wherein the optimization of said at least one physiologic function has a result that improves at least one life factor selected from the group consisting of: general sense of well-being, clinical depression, fatigue sensation, athletic activity, positive interaction with another organism, motivation, liveliness and healing rate.

46. The composition according to claim 1 further comprising at least one promoter of mitochondrial metabolism.

47. The composition according to claim 46 wherein the facet of mitochondrial metabolism that is promoted is selected from the group consisting of: oxidative phosphorylation, coupling efficiency (energy versus heat production), free radical generation, free radical scavenging, initiation of apoptosis, mtDNA transcription, mtDNA maintenance, generation of reactive oxygen species, controlling DNA acetylation, controlling DNA methylation, histone modification, mitochondrial protein translation, post translational modification or mitochondrial proteins, mitochondrial protein import or translocation, ion import, ion homeostasis, nucleotide translocation, ATP translocation, mitochondrial fission, mitochondrial fusion, Ca⁺⁺ compartmentalization or homeostasis, steroid biosynthesis, a component of the urea cycle, fatty acid oxidation, a component of the tricarboxylic acid cycle, pyruvate metabolism, cellular redox balance, synthesis of precursor compounds for a mitochondrial function or activity, iron metabolism, oxygen metabolism and any component or activity of the electron transport chain.

48. The composition according to claim 1 further comprising a substance selected from the group consisting of: Riboflavin (B2), L-Creatine, CoQ₁₀, L-arginine, L-carnitine, vitamin C, cyclosporin A, manganese, magnesium, carnosine, vitamin E, resveratrol, α-lipoic acid, folinic acid, dichloroacetate, succinate, prostaglandins (PG) prostacyclins, thromboxanes, prostanoic acid, 2-arachidonoylglycerol and glutathione.

49. The composition according to claim 1 wherein the substance is complexed by covalent or non-covalent bonding with non-hormonal or non-prohormonal material.

50. The composition according to claim 1 wherein the substance further comprises at least one lipophilic vitamin.

51. The composition according to claim 50 wherein the at least one lipophilic vitamin is selected from the group consisting of: vitamin A, vitamin D, vitamin E and vitamin K.

52. The composition according to claim 1 wherein the substance is administered using a device implanted sub-dermally in the non-human animal.

53. The composition according to claim 52 wherein the composition is prepared using a 3D printing method.

54. A method for improving a non-human animal's well-being comprising: delivering to a selected non-human animal a dosage of androgen hormone or prohormone selected to improve at least one physiologic function in the selected animal.

55. The method according to claim 54 wherein:
a) testosterone level is assessed in a non-human animal selected for possible benefit;
b) an amount of testosterone is selected to increase circulating testosterone to a desired level and to improve at least one facet of the select animal's physiology;
c) at least one composition is prepared, said composition comprising testosterone in an amount that taking into account the frequency and volume of said composition is designed to administer the amount selected in b); and
d) administering said at least one composition in accordance with the volume and frequency of c).

56. The method according to claim 54 wherein said at least one physiologic function is selected from the group consisting of: adipose metabolism, cardiac performance, glucose utilization, circulation, general nervous system activation or activity, hormonal secretion, salt (electrolyte) balance, function of a particular tissue or organ system, membrane transport across the membrane of one or more cell types, muscle activity, maintained muscle mass, O₂ consumption, skin health and visual abilities.

57. The method according to claim 55 wherein after at least two weeks have elapsed from initiation of part d), parts a) through d) are repeated with an outcome that a desired androgen concentration remains is circulation.

58. The method according to claim 54 further comprising: delivering to said selected animal a promoter of mitochondrial function that is also selected to improve at least one physiologic function in the selected animal, said at least one physiologic function being identical to or differing from the at least one physiologic function targeted by the hormone or prohormone.

59. The method according to claim 58 wherein subsequent to mitochondrial function in said animal being assessed, said method further comprises choosing a substance to improve said mitochondrial function, and administering said substance to said animal.

60. The method according to claim 56 further comprising assessing mitochondrial function in said animal, choosing a substance to improve said mitochondrial function, and administering said substance to said animal.

61. The method according to claim 54 wherein the at least one composition comprises one selected from the group consisting of: beef, elk, chicken, salmon, protein binder and a vitamin.

62. The method according to claim 54 wherein the at least one composition comprises one in a form selected from the group consisting of: a capsule, a tablet, a caplet, a liquid beverage, a powder, a liquid or powder beverage additive, a gel, a ready-to-eat food, either moist or dry, a chunk, a bar and a toy.

63. A method for improving well-being in a non-human animal, said method comprising increasing circulating levels of SHBG protein in the blood.

64. The method according to claim 63 wherein SHBG is produced by enteric organisms cultured into the animal's microbiome.

65. The method according to claim 63 wherein a SHBG porous capsule is introduced subcutaneously, said capsule comprising in its interior an expression system producing SHBG.

66. The method according to claim 63 wherein the animal is genetically modified to increase SHBG expression.

67. The method according to claim 63 comprising a lipoporous capsule is introduced subcutaneously, said capsule comprising in its interior a system releasing androgenic substance capable of binding with SHBG.

68. The method of claim 54 further comprising delivering to said animal a compound having or supporting cannabinolic activity.

69. A method for improving a non-human animal's well-being comprising administering to said animal at least one mitochondrial metabolic booster selected from the group consisting of: Riboflavin (B2), L-Creatine, CoQ₁₀, L-arginine, L-carnitine, vitamin C, cyclosporin A, manganese, magnesium, carnosine, vitamin E, resveratrol, α-lipoic acid, folinic acid, dichloroacetate, succinate, prostaglandins (PG) prostacyclins, thromboxanes, prostanoic acid, and glutathione in association with administering to said animal at least one compound having or supporting cannabinolic activity.

70. A method for improving a non-human animal's well-being comprising: administering to said animal at least one composition designed to impact a system or output selected from the group consisting of: oxidative phosphorylation, coupling efficiency (energy versus heat production), free radical generation, free radical scavenging, initiation of apoptosis, mtDNA transcription, mtDNA maintenance, generation of reactive oxygen species, controlling DNA acetylation, controlling DNA methylation, histone modification, mitochondrial protein translation, post translational modification or mitochondrial proteins, mitochondrial protein import or translocation, ion import, ion homeostasis, nucleotide translocation, ATP translocation, mitochondrial fission, mitochondrial fusion, Ca⁺⁺ compartmentalization or homeostasis, steroid biosynthesis, a component of the urea cycle, fatty acid oxidation, a component of the tricarboxylic acid cycle, pyruvate metabolism, cellular redox balance, synthesis of precursor compounds for a mitochondrial function or activity, iron metabolism, oxygen metabolism and any component or activity of the electron transport chain in said animal; and balancing said impact through administering to said animal at least one compound having or supporting cannabinolic activity.

71. A method for improving a non-human animal's well-being comprising administering to said animal at least one compound having or supporting cannabinolic activity for rebalancing at least one physiologic system in said animal and wherein said animal has been recognized as one as likely to benefit from such rebalancing.

72. The method of claim 71 wherein said at least one compound comprises one or more compounds selected from the group consisting of: CBGA, CBGAM, CBG, CBGM; CBGVA and CBGV.

73. The composition of claim 2 comprising a cannabinoid selected from the group consisting of: CBCA, CBC, CBCVA, CBCV, CBDA, CBD, CBDM, CBD-C4, CBDVA, CBDV, CBD-C1, THCA-A, THCA-B, 6a,10a-trans-6a,7,8,10a-tetrahydro-6,6,9-trimethyl-3-pentyl-6H-dibenzo[b,d]pyran-1-ol, THC,) THCA-C4, THC-C4, THCVA, THCV, Δ⁷-cis-isotetrahydro-cannabivarin, THCA-C1, THC-C1, Δ⁸-TCA, Δ⁸-THC, CBL, CBLA, CBLV, CBEA-A, CBEA-B, CBE, CBNA, CBN, CBNM, CBN-C4, CBV, CBN-C2, CBN-C1), CBND, CBDV,CBT, 10-EHDT, 8,9-DHDT, CBTV, CBTVE, DCBF, CBF, CBCN, CBT, OTHC, cis-THC, 2H-iso-HHCV, CBR, triOH-THC, abinene, α-pinene, 4,8-dimethyl-1,7-nonadien-4-ol, 2-hydroxy-4-methyl-valeric, acid, methyl, ester, octanal, O-cymene, eucalyptol, α-phellandrene, cis-sabinene, hydroxide, myrcenol, terpinen-4-ol, α-terpineol, β-thujene, ç-terpinene, trans-α-ocimene, carveol, β-citral, guanidine, geraniol, bornyl, acetate, β-pinene, thymol, geranic, acid, methyl, ester, α-terpinyl, acetate, d-limonene, eugenol, geranyl, acetate, dihydrocarvyl, acetate, α-ylangene, cis-dodec-5-enal, 3-phenyl-2-propenoic, acid, methyl, ester, β-elemene, c, vanillin, epoxy-α-terpenyl, acetate, butanoic, acid, 2-methyl-, 3,7-dimethyl-2,6-octadienyl, ester, 1-methyl-4-(1-acetoxy-1-methylethyl)-cyclohex-2-enol, 1,2,3,4,4a,5,6,8a-octahydro-4a,8-dimethyl-2-(1-methylethenyl)-, [2r-(2àa,4aà,8aà)]-naphthalene, p-mentha-1(7),8-dien-2-ol, ç-muurolene, hydroxy-α-terpenyl, acetate, nerolidol, geranyl, bromide, (−)-α-panasinsen, pyrocatechol, ç-elemene, 9,10-dehydro-isolongifolene, à-calacorene, cis-verbenol, acetic, acid, 1-methyl-1-(4-methyl-5-oxo-cyclohex-3-enyl)ethyl, ester, alloaromadendrene, z,z-2,6-dimethyl-3,5,7-octatriene-2-ol, 4-epi-cubedol, 2-oxabicyclo[2.2.2]octan-6-ol, 1,3,3-trimethyl-acetate, patchoulane, farnesol, caryophyllene, oxide, cis-lanceol, ledene, oxide-(ii), farnesol, acetate, 6-epi-shyobunol, falcarinol, phytol, aromadendrene, oxide-(2), heptacosane, longipinene, epoxide, hentriacontane, decamethyl-cyclopentasiloxane, geranyl, isobutyr, hexamethyl-cyclotrisiloxane, 1-docosene, tetratetracontane, dodecamethyl-cyclohexasiloxane, URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A,KML29, MAFP, MJN110, ML30, N-arachidonoyl maleimide, OL-135, OL92, PF-04457845, SA-57, ST4070, URB880, URB937, indomethacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen, TFAP, 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)indomethacin amide, indomethacin heptyl ester, SC236, sulinac, sulindac sulfide, pravadoline, naproxen, naproxen sodium salt, meclofenamate sodium, ibuprofen, S-ibuprofen, piroxicam, ketoprofen, S-ketoprofen, R-ibuprofen, Ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1H-indole-2,3-dione, aceclofenac, acetylsalicylic acid-d4, S-ibuprofen lysinate, loxoprofen, CAY10589, ZLJ-6, isoicam, dipyrone, YS121 and MEG (mercaptoethylguanidine).
